(19) United States
(12) Patent Application Publication
(10) Pub. No.: US 2021/0130384 A1
(43) Pub. Date: May 6, 2021

(54) PROCESS FOR PRODUCING 2'-O-FUCOSYLLACTOSE

(71) Applicant: BASF SE, Ludwigshafen am Rhein (DE)

(72) Inventors: MICHAEL PUHL, Ludwigshafen am Rhein (DE); Wolfgfang SIEGEL, Ludwigshafen am Rhein (DE); Stephanie RENZ, Ludwigshafen am Rhein (DE); Andreas WÖLFERT, Ludwigshafen am Rhein (DE)

(21) Appl. No.: 16/074,168

(22) PCT Filed: Feb. 2, 2017
(86) PCT No.: PCT/EP2017/052280

§ 371 Date: Jul. 31, 2018

(30) Foreign Application Priority Data
Feb. 3, 2016 (EP) 16154115.6

(51) Int. Cl. C07H 1/06; C07H 15/18; C07H 3/06

(52) U.S. Cl. CPC C07H 1/06; C07H 3/06; C07H 15/18

(57) ABSTRACT

The present invention relates to a method for preparing 2′-O-fucosyllactose, the intermediates obtainable by this method and the use of these intermediates. The preparation comprises the reaction of a protected fucose of the general formula (I) with a tri(C₁-C₆-alkyl)silyl iodide to give a protected 1-iodofucose followed by the reaction of the protected 1-iodofucose with a compound of the general formula (II), in the presence of at least one base and deprotecting the resulting coupling product to afford 2′-O-fucosyllactose.

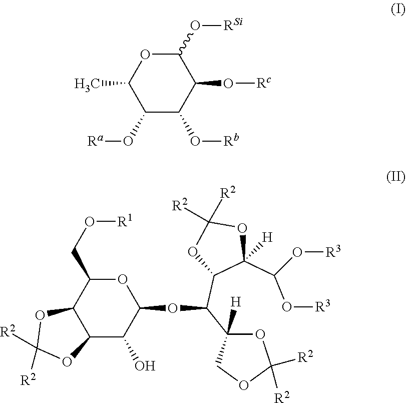

In this context, the variables are each defined as follows:
R^{a} and R^{b} are the same or different and are —C(═O)—C₁-C₆-alkyl, or —C(═O)-phenyl, wherein said phenyl is unsubstituted or optionally has 1 to 5 substituents, or R^{a} and R^{b} together are a radical —(C═O)— or a substituted methylene radical —C(R^{d}R^{e})—,
R^{c} is a radical R^{Si} or is benzyl, wherein said benzyl is unsubstituted or optionally has 1, 2 or 3 substituents,
R^{Si} is a radical of the formula SiR^{f}R^{g}R^{h}, where R^{f}, R^{g} and R^{h} are the same or different and are C₁-C₈-alkyl for example,
R¹ is a radical —C(═O)—R¹¹ or a radical SiR¹²R¹³R¹⁴
R² are the same or different and are C₁-C₈-alkyl for example;
R³ are the same or different and are for example C₁-C₈-alkyl or both radicals R³ together form a linear C₁-C₄-alkenyl, which is unsubstituted or has 1 to 6 methyl groups as substituents.

The present invention relates to a method for preparing 2′-O-fucosyllactose, the intermediates obtainable by this method and the use of these intermediates.

### BACKGROUND OF THE INVENTION

2′-O-Fucosyllactose (CAS No. 41263-94-9: α-L-fucopyranosyl)-(1→2)-O-β-D-galactopyranosyl-(1→4)-D-glucopyranose) is an oligosaccharide, which is found in relatively large quantities in breast milk. It has been variously reported that the 2′-O-fucosyllactose present in breast milk causally reduces the risk of infection in newborns who are breast fed (see e.g. Weichert et al., Nutrition Research, 33 (2013), Volume 10, 831-838; Jantscher-Krenn et al., Minerva Pediatr. 2012, 64 (1) 83-99; Morrow et al., J. Pediatr. 145 (2004) 297-303). 2′-O-Fucosyllactose is therefore of particular interest as a constituent of food supplements, particularly as additive for humanized milk products, especially for infant nutrition.

The preparation of 2′-O-fucosyllactose by classical chemical or biochemical means has been variously described in the literature (see e.g. Carbohydrate Res. 88(1) (1981) 51, Carbohydrate. Res. 154 (1986) 93-101, Carbohydrate. Res. 212 (1991) C1-C3, J. Org. Chem. (1997) 62, 992, Heterocycles 84(1) (2012) 637, U.S. Pat. No. 5,438,124, WO 2010/115934, WO 2010/115935, WO 2010/070616, WO 2012/113404 and WO 2013/48294). The chemical preparation is typically based on fucosylation of suitably protected acceptors, i.e. lactose derivatives partially protected, unprotected at the 2-position, which bear a thioalkyl group, an alkenyloxy group, a trichloroacetimidate or a bromine atom in place of the anomeric OH group, e.g. 4-O-(6-O-acetyl-3,4-isopropylidene-β-D-galactopyranosyl)-2,3; 5,6-bis-O-isopropylidene-D-glucose dimethylacetal, by using activated fucosyl donors such as methyl 1-thio-2,3,4-tri-O-benzyl-β-L-fucopyranoside, methyl 3,4-O-isopropylidene-2-O-(4-methoxybenzyl)-1-thio-L-fucopyranoside, pentenyl 3,4-O-isopropylidene-2-O-(4-methoxybenzyl)-β-L-fucopyranoside, phenyl 1-thio-2,3,4-tri-O-benzyl-β-L-fucopyranoside, 2,3,4-tri-O-benzyl-β-L-fucopyranosyl bromide, or 2,3,4-tri-O-benzyl-β-L-fucopyranosyl trichloracetimidate (with respect to fucose donors see the literature cited above and Tetrahedron Lett. 31 (1990) 4325) A disadvantage is the complex, generally multistage preparation, of the fucosyl donors. Another disadvantage is found to be that these fucosyl donors cannot be provided in industrial amounts and/or are not stable on storage due to their reactive group at the anomeric center.

For instance, R. K. Jain et al., Carbohydrate Research, 212 (1991), pp. C1-C3 describe a route for the preparation of 2′-O-fucosyllactose by fucosylation of 4-O-(6-O-acetyl-3,4-isopropylidene-β-D-galactopyranosyl)-2,3; 5,6-bis-O-isopropylidene-D-glucose dimethylacetal using methyl 3,4-O-isopropylidene-2-O-(4-methoxybenzyl)-1-thio-β-L-fucopyranoside or pentyl 3,4-O-isopropylidene-2-O-(4-methoxybenzyl)-β-L-fucopyranoside as fucosylating reagents. These fucosylating reagents are, however, complex to prepare. A similar synthesis is described in J. Org. Chem. (1997) 62, 992.

WO 2010/115934 and WO 2010/115935 describe the preparation of 2-fucosyllactose using 2-O-benzylated fucosyl donors. The fucosyl donors are complex to prepare and in some cases have reactive groups at their anomeric center which consequently have low storage stability. Moreover, for their efficient reaction with the lactose derivatives, toxic and corrosive reagents generally have to be used, such as Lewis acids, trifluoromethanesulfonic acid, mercury salts or bromine. A similar method is known from WO 2010/070616.

WO 2012/113404 describes, inter alia, O-protected fucosyl phosphites, which may be used as fucosyl donors in glycosylations. Here also, the 2,3,4-O-protected fucose derivatives must first be prepared in multistage reactions which are subsequently reacted with phosphorus(III) trichloride and a phenol to the corresponding fucosyl phosphite.

In summary, the methods known to date for preparing 2′-O-fucosyllactose are complex and therefore uneconomic and for which ecologically questionable reagents are used. In addition, the fucosyl donors used in these methods are often not stable on storage and/or cannot be provided in industrial amounts. Furthermore, the 2′-O-fucosyllactose obtained by the methods known to date have impurities which cannot be completely removed, in particular heavy metals, and also trisaccharides such as β-2′-O-fucosyllactose (=β-L-fucopyranosyl)-(1→2)-O-β-D-galactopyranosyl-(1→4)-D-glucopyranose). These impurities are particularly problematic when used in human nutrition.

### SUMMARY OF THE INVENTION

It is an object of the present invention to provide a method for preparing 2′-O-fucosyllactose which does not have the problems of the prior art. The method should in particular allow the use of starting materials that can be easily prepared, in particular readily available fucosyl donors that are stable on storage. The method should furthermore ensure good yields and good stereoselectivities in the fucosylation without expensive and/or ecologically questionable reagents having to be used. In addition, the method should be suitable so as to largely avoid the removal of any protecting groups by hydrogenolysis over transition metal catalysts.

It has been found that, by reacting a protected fucose of the general formula (I),

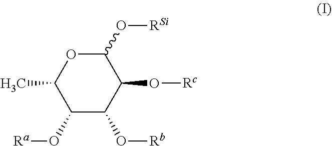

in which
R^{a} and R^{b} are the same or different and are —C(═O)—C₁-C₆-alkyl, —C(═O)-phenyl, wherein phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, or benzyl, wherein said benzyl is unsubstituted or optionally has 1, 2 or 3 substituents selected from halogen, C₁-C₄-alkyl or C₁-C₄-alkoxy, or
R^{a} and R^{b} together are a carbonyl radical —(C═O)— or a substituted methylene radical —C(R^{d}R^{e})—, wherein R^{d} and R^{e} are the same or different and are selected from hydrogen, phenyl and C₁-C₄-alkyl or both radicals R^{d} and R^{e} together are linear C₄-C₆-alkenyl,
R^{c} is a radical R^{Si} or benzyl, wherein said benzyl is unsubstituted or optionally has 1, 2 or 3 substituents selected from halogen, C₁-C₄-alkyl or C₁-C₄-alkoxy, and
R^{Si} may be the same or different and is a radical of the formula SiR^{f}R^{g}R^{h}, wherein R^{f}, R^{g} and R^{h} are the same or different and are selected from C₁-C₈-alkyl, C₃-C₈-cycloalkyl, phenyl and C₃-C₈-cycloalkyl-C₁-C₄-alkyl,

with a tri(C₁-C₆-alkyl)silyl iodide and subsequently reacting the fucose donor thus obtained, i.e. the corresponding 1-iodofucose, with a suitable lactose acceptor, namely the compound of the general formula (II) defined in more detail below, in the presence of at least one base, a corresponding protected 2¹-O-fucosyllactose derivative of the general formula (III) is obtained in good yields and high selectivity, which can then be deprotected in a manner known per se to obtain 2′-O-fucosyllactose.

Accordingly, the invention firstly relates to a method for preparing 2′-O-fucosyllactose, comprising the steps of
a) reacting a protected fucose of the general formula (I),

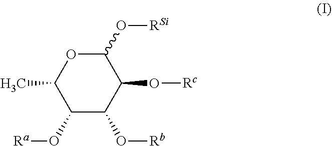

in which
R^{a} and R^{b} are the same or different and are —C(═O)—C₁-C₆-alkyl, —C(═O)-phenyl, wherein phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, or benzyl, wherein said benzyl is unsubstituted or optionally has 1, 2 or 3 substituents selected from halogen, C₁-C₄-alkyl or C₁-C₄-alkoxy, or
R^{a} and R^{b} together are a carbonyl radical —(C═O)— or a substituted methylene radical —C(R^{d}R^{e})—, wherein R^{d} and R^{e} are the same or different and are selected from hydrogen, phenyl and C₁-C₄-alkyl or both radicals R^{d} and R^{e} together are linear C₄-C₆-alkenyl,
R^{c} is a radical R^{Si} or benzyl, wherein said benzyl is unsubstituted or optionally has 1, 2 or 3 substituents selected from halogen, C₁-C₄-alkyl or C₁-C₄-alkoxy, and
R^{Si} may be the same or different and is a radical of the formula SiR^{f}R^{g}R^{h}, wherein R^{f}, R^{g} and R^{h} are the same or different and are selected from C₁-C₈-alkyl, C₃-C₈-cycloalkyl, phenyl and C₃-C₈-cycloalkyl-C₁-C₄-alkyl,
with a tri(C₁-C₆-alkyl)silyl iodide to give a protected 1-iodofucose of the general formula (I.a)

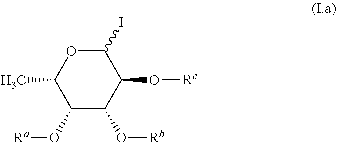

wherein R^{a}, R^{b} and R^{c} have the definitions stated above;
b) reacting the protected 1-iodofucose of the general formula (I.a) obtained in step a) with a compound of the general formula (II),

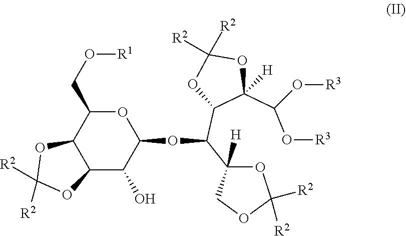

in which
R¹ is a radical —C(═O)—R¹¹ or a radical SiR¹²R¹³R¹⁴, in which
   R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄haloalkoxy, and
   R¹², R¹³ and R¹⁴ are the same or different and are selected from C₁-C₈-alkyl, C₃-C₈-cycloalkyl, phenyl and C₃-C₈-cycloalkyl-C₁-C₄-alkyl,
   or
   is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl;
R² may be the same or different and are C₁-C₈-alkyl or two radicals R² attached to the same carbon atom together form a linear C₃-C₆-alkenyl, which is unsubstituted or has 1 to 6 methyl groups as substituents;
R³ may be the same or different and are C₁-C₈-alkyl or both radicals R³ together form a linear C₁-C₄alkenyl, which is unsubstituted or has 1 to 6 methyl groups as substituents;
in the presence of at least one base;
c) deprotecting the coupling product of the general formula (III) obtained in step b)

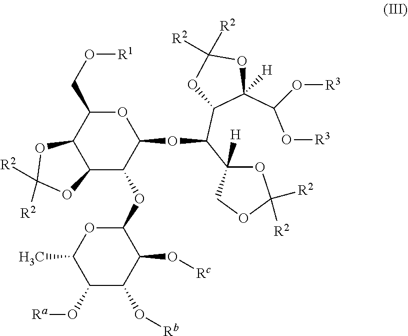

where R^{a}, R^{b}, R^{c}, R¹, R² and R³ are as defined above;
to obtain 2′-O-fucosyllactose.

The invention further relates to the protected and the partially protected 2′-O-fucosyllactose derivatives of the general formulae (IIIa), (IIIb), (IVa) and (IVb);

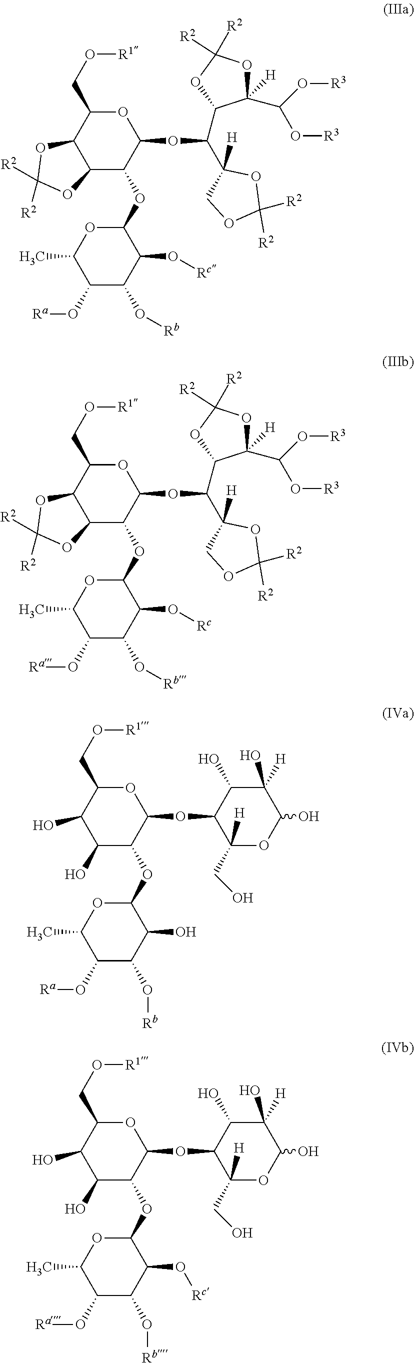

in which:
R^{a} and R^{b} have the definitions stated above, R^{a}′″ and R^{b}′″ together are a carbonyl radical —(C═O)— or a substituted methylene radical —C(R^{d}R^{e})—, wherein R^{d} and R^{e} are the same or different and are selected from hydrogen, phenyl and C₁-C₄-alkyl or both radicals R^{d} and R^{e} together are linear C₄-C₆-alkenyl,
R^{a}′″ and R^{b}′″ together are a carbonyl radical —(C═O)—,
R^{c} is as defined above,
R^{c}′ is benzyl which is unsubstituted or optionally has 1, 2 or 3 substituents selected from halogen, C₁-C₄-alkyl or C₁-C₄-alkoxy,
R^{c}″ is hydrogen or a radical R^{Si},
R¹″ is hydrogen, a radical —C(═O)—R¹¹ or a radical SiR¹²R¹³R¹⁴, in which
   R¹¹ is hydrogen, C₁-C₈-alkyl, C₁-C₈-haloalkyl, C₃-C₈-cycloalkyl, C₃-C₈-cycloalkyl-C₁-C₈-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
   R¹², R¹³ and R¹⁴ are the same or different and are selected from C₁-C₈-alkyl, C₃-C₈-cycloalkyl, phenyl and C₃-C₈-cycloalkyl-C₁-C₄-alkyl,
   or
   is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl,
R¹′″ is hydrogen, a radical —C(═O)—R¹¹, where
   R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy,
   or
   is benzyl, wherein said benzyl is unsubstituted or optionally has 1, 2 or 3 substituents selected from halogen, C₁-C₄-alkyl, C₁-C₄-alkoxy or
   —O—C(═O)—C₁-C₄-alkyl, and
R² and R³ have the definitions stated above.

The invention further provides the protected fucose derivatives of the general formula (I′),

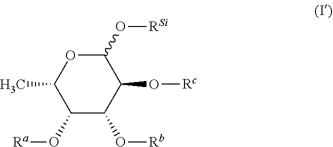

where R^{a}, R^{b}, R^{c} and R^{Si} have the definitions stated above, wherein the radicals R^{a}, R^{b} and R^{c} are not all three simultaneously benzyl or 4-methoxybenzyl.

The invention further provides the protected fucose derivatives of the general formula) (I.a′)

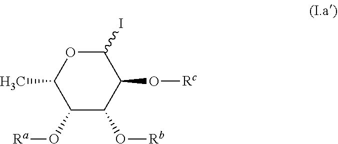

where R^{a}, R^{b}, R^{c} and R^{Si} have the meanings stated above, wherein the radicals R^{a}, R^{b} and R^{c} are not all three simultaneously benzyl and, in the case that R^{a} and R^{b} together form a dimethylmethylene radical —C(CH₃CH₃)—, R^{c} is not a tert-butyldimethylsilyl radical.

The inventive method is linked to a series of advantages. The fucosyl donors of the formula (I) are stable on storage and are accessible in industrial amounts. A particular advantage of the method according to the invention is that the fucosyl donors of the formula (I), via the protected 1-iodofuscoses of the general formula (I.a), can be reacted in a simple manner with the lactose derivatives of the formula (II) to give the protected 2′-O-fucosyllactoses of the general formula (III) without having to use expensive and/or ecologically harmful reagents. The reagents used in the present method are available in sufficient amount for industrial syntheses, in contrast to the reagents used typically in conventional methods, such as trichloroacetonitrile, BF₃ etherate, N-iodosuccinimide and trifluoromethansuifonic anhydride. In addition, the synthesis of the protected 2′-O-fucosyllactoses of the general formula (III) using these reagents is achieved without forming residues or by-products that are difficult to remove and/or harmful to health. The method affords the primary coupling products of the formula (III) in good yields and good stereoselectivity relative to the glycosylation. The protecting groups are removed from the compounds of the formula (III) predominantly under mild basic and/or acidic hydrolysis conditions and also optionally hydrogenolytically. The intermediates arising of the formula (III), particularly of the formulae (IIIa) and (IIIb), and also the partially protected intermediates of the formula (IV), particularly of the formulae (IVa) and (IVb), are stable, in particular stable on storage, and can be purified. In addition, the method can readily be carried out on a relatively large scale. A further advantage of the method according to the invention is that, in particular, the undesired β-isomer is not formed, or is formed to a very much lower extent, than in the methods of the prior art. For instance, in the reaction of compound (I) with compound (II), the undesired β-isomer of compound (III) is generally formed in such low amounts that the ratio of β-isomer to a-isomer is not more than 1:10 and, for example, is in the range from 1:10 to 1;40. The method according to the invention therefore enables the desired 2′-O-fucosyllactose to be prepared, optionally after purification, having a content of β-isomer of less than 1%, in particular less than 0.5%.

The method and the reactants of the formulae (I′) and (I.a′) obtained by the method and also the intermediates of the formulae (IIIa), (IIIb), (IVa) and (IVb) are, therefore, particularly suitable for preparing 2′-O-fucosyllactose. Accordingly, the present invention also relates to the use of compounds of the general formulae (I′) and (I.a′) for preparing 2′-O-fucosyllactose and also the use of compounds of the general formulae (IIIa), (IIIb), (IVa) or (IVb) for preparing 2′-O-fucosyllactose.

The quality of the 2′-O-fucosyllactose obtained by the method according to the invention renders it particularly suitable for preparing foodstuffs. Accordingly, the present invention also relates to
the use of at least one of the compounds of the general formulae (I′), (I.a′), (IIIa), (IIIb), (IVa) or (IVb) for preparing foodstuffs and food additives, comprising the preparation of 2′-O-fucosyllactose from at least one of the compounds of the general formulae (I′), (I.a′), (IIIa), (IIIb), (IVa) or (IVb);
a method for preparing foodstuffs comprising the preparation of 2′—O-fucosyllactose from at least one of the compounds of the general formulae (I′), (I.a′), (IIIa), (IIIb), (IVa) or (IVb) and formulation of the 2′-O-fucosyllactose thus obtainable in a foodstuff.

### DETAILED DESCRIPTION OF THE INVENTION:

In the context of the present invention, the terms used generically are defined as follows:

The prefix Cₓ₋C_{y} denotes the number of possible carbon atoms in the particular case.

The term “halogen” in each case denotes fluorine, bromine, chlorine or iodine, specifically fluorine, chlorine or bromine.

The term “C₁-C₄-alkyl” denotes a linear or branched alkyl radical comprising 1 to 4 carbon atoms, such as methyl, ethyl, propyl, 1-methylethyl (isopropyl), butyl, 1-methylpropyl (sec-butyl), 2-methylpropyl (isobutyl) or 1,1-dimethylethyl (tert-butyl).

The term “C₁-C₆-alkyl” denotes a linear or branched alkyl radical comprising 1 to 6 carbon atoms. In addition to the radicals mentioned for C₁-C₄-alkyl, examples are n-pentyl, n-hexyl, 2-pentyl, 2-hexyl, 3-pentyl, 3-hexyl, 2,2-dimethylpropyl, 2-methylbutyl, 3-methylbutyl, 2-ethylbutyl, 3-ethylbutyl, 2-methylpentyl, 3-methylpentyl or 4-methylpentyl.

The term “C₁-C₈-alkyl” denotes a linear or branched alkyl radical comprising 1 to 8 carbon atoms. In addition to the radicals mentioned for C₁-C₆-alkyl, examples are n-heptyl, n-octyl, 2-heptyl, 2-octyl, 3-heptyl, 3-octyl, 2-ethylpentyl, 3-ethylpentyl, 4-ethyl-pentyl, 2-ethylhexyl and positional isomers thereof.

The term “C₁-C₈-haloalkyl” denotes a linear or branched alkyl radical comprising 1 to 8 carbon atoms, particularly 1 to 4 carbon atoms (C₁-C₄-haloalkyl), in which one or more or all hydrogen atoms have been replaced by halogen atoms, in particular by fluorine or chlorine atoms. Examples for this purpose are chloromethyl, dichloromethyl, trichloromethyl, fluoromethyl, difluoromethyl, trifluoromethyl, chlorofluoromethyl, dichlorofluoromethyl, chlorodifluoromethyl, 2,2-difluoroethyl, 2,2,2-trifluoroethyl, 1,1,2,2-tetrafluoroethyl, pentafluoroethyl, 2,2-difluoropropyl, 3,3-difluoropropyl, 3,3,3-trifluoropropyl, 2,2,3,3,3-pentafluoropropyl, heptafluoropropyl, and the like.

The term “C₁-C₄-alkoxy” denotes straight-chain or branched saturated alkyl groups comprising 1 to 4 carbon atoms which are bonded via an oxygen atom. Examples of C₁-C₄-alkoxy are methoxy, ethoxy, n-propoxy, 1-methylethoxy (isopropoxy), n-butoxy, 1-methylpropoxy (sec-butoxy), 2-methylpropoxy (isobutoxy) and 1,1-dimethylethoxy (tert-butoxy).

The term “C₁-C₄-haloalkoxy” denotes straight-chain or branched saturated haloalkyl groups comprising 1 to 4 carbon atoms which are bonded via an oxygen atom. Examples in this case are fluoromethoxy, difluoromethoxy, trifluoromethoxy, 1-fluoroethoxy, 2-fluoroethoxy, 2,2-difluoroethoxy, 2,2,2-trifluoroethoxy, 1,1,2,2-tetrafluoroethoxy, pentafluoroethoxy, 3,3,3-trifluoroprop-1-oxy, 1,1,1-trifluoroprop-2-oxy, 1-fluorobutoxy, 2-fluorobutoxy, 3-fluorobutoxy, 4-fluorobutoxy and the like.

The term “C₃-C₈-cycloalkyl” denotes a cyclic, saturated hydrocarbyl radical comprising 3 to 8 carbon atoms. Examples are cyclopropyl, cyclobutyl, cyclopentyl, cyclohexyl, cycloheptyl and cyclooctyl.

The term “C₃-C₈-cycloalkyl-C₁-C₄-alkyl” denotes a linear or branched alkyl radical comprising 1 to 4 carbon atoms, in which one hydrogen atom has been replaced by C₃-C₈-cycloalkyl, as defined above.

The term “linear C₁-C₄-alkenyl” denotes a linear, divalent hydrocarbyl radical having 1 to 4 carbon atoms, such as methylene, ethane-1,2-diyl, propane-1,3-diyl, and butane-1,4-diyl.

The term “linear C₄-C₆-alkenyl” denotes a linear, divalent hydrocarbyl radical having 4 to 6 carbon atoms, such as butane-1,4-diyl, pentane-1,5-diyl and hexane-1,6-diyl.

The term “linear C₃-C₆-alkenyl” denotes a linear, divalent hydrocarbyl radical having 3 to 6 carbon atoms, such as propane-1,3-diyl, butane-1,4-diyl, pentane-1,5-diyl and hexane-1,6-diyl.

The term “foodstuff” or “food” denotes compositions and formulations which are intended and suitable as nutrition for mammals, particularly for human beings. In the context of the present invention, they include both compositions based on naturally-occurring products, e.g. dairy products, and also artificially prepared formulations, for example, for dietary or medicinal nutrition, which can be used directly or optionally have to be converted into a ready-to-use formulation before use by addition of liquid.

The term “food additive” denotes substances which are mixed with the foodstuff to achieve chemical, physical or also physiological effects.

With respect to the method according to the invention and the compounds of the formulae (I), (I′), (I.a), (I.a′), (III), (IIIa), (IV) and (IVa), the variables R^{a} and R^{b} within one formula preferably have the same definition in each case.

With respect to the method according to the invention and the compounds of the formulae (IIIb′″), (IIIc), (IIIe), (IVa′) and (IVb′), the variables R^{a}″ and R^{b}″ within one formula preferably have the same definition in each case.

In a first preferred embodiment of the present invention, the variables R^{a} and R^{b} in the compounds of the formulae (I), (I′), (I.a), (I.a′), (III), (IIIa), (IV) and (IVa) are —C(═O)-C₁-C₆-alkyl, —C(═O)-phenyl, wherein phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, or the variables R^{a} and R^{b} together are a carbonyl radical —(C═O)—.

In a second preferred embodiment of the present invention, the variables R^{a} and R^{b} in the compounds of the formulae (I), (I′), (I.a), (I.a′), (III), (IIIa), (IV) and (IVa) are benzyl, wherein said benzyl is unsubstituted or optionally has 1, 2 or 3 substituents selected from halogen, C₁-C₄-alkyl or C₁-C₄-alkoxy.

In a third preferred embodiment of the present invention, the variables R^{a} and R^{b} in the compounds of the formulae (I), (I′), (I.a), (I.a′), (III), (IIIa), (IV) and (IVa) are a substituted methylene radical —C(R^{d}R^{e})—, where R^{d} and R^{e} are the same or different and are selected from hydrogen, phenyl and C₁-C₄-alkyl or both radicals R^{d} and R^{e} together are linear C₄-C₆-alkenyl.

The variables R^{a} and R^{b} in the compounds of the formulae (I), (I.a), (I.a′), (III), (IIIa), (IV) and (IVa) are particularly preferably acetyl, pivaloyl, benzoyl, 4-chlorobenzoyl, 4-fluorobenzoyl, 4-methylbenzoyl, benzyl, 4-methoxybenzyl, 4-chlorobenzyl, 4-methylbenzyl, 2-chlorobenzyl or 2,4-dichlorobenzyl, or the variables R^{a} and R^{b} together are a carbonyl radical —(C═O)— or a substituted methylene radical —C(R^{d}R^{e})—, where R^{d} and R^{e} are identical and are selected from hydrogen and methyl.

The variables R^{a} and R^{b} in the compounds of the formulae (I), (I′), (I.a), (I.a′), (III), (IIIa), (IV) and (IVa) are especially acetyl, benzoyl, 4-chlorobenzoyl, 4-fluorobenzoyl, 4-methylbenzoyl, benzyl, 4-methoxybenzyl, 4-chlorobenzyl, 4-methylbenzyl, 2-chlorobenzyl or 2,4-dichlorobenzyl, or the variables R^{a} and R^{b} together are isopropylidene.

The variables R^{a}′ and R^{b}′ in the compounds of the formulae (IIIb″) and (IIId′) together are preferably a substituted methylene radical —C(R^{d}R^{e})—, where both radicals R^{d} and R^{e} are selected from hydrogen, phenyl and methyl or both radicals R^{d} and R^{e} together are propane-1,4-diyl.

The variables R^{a}′ and R^{b}′ din the compounds of the formulae (IIIb″) and (IIId′) together are especially isopropylidene.

The variables R^{a}″ and R^{b}″ in the compounds of the formulae (IIIb′″), (IIIc), (IIIe), (IVa′) and (IVb′) are preferably both benzyl, wherein said benzyl is unsubstituted or optionally has 1 or 2 substituents selected from fluorine, chlorine, bromine, methyl and methoxy.

The variables R^{a}″ and R^{b}″ in the compounds of the formulae (IIIb′″), (IIIc), (IIIe), (IVa′) and (IVb′) are both especially benzyl, 4-chlorobenzyl, 4-methylbenzyl, 4-methoxy-benzyl, 2-chlorobenzyl and 2,4-dichlorobenzyl.

The variables R^{a}′″ and R^{b}′″ in the compounds of the formulae (IIIb) together are preferably a carbonyl radical —(C═O)— or a substituted methylene radical —C(R^{d}R^{e})—, where both radicals R^{d} and R^{e} are selected from hydrogen, phenyl and methyl or both radicals R^{d} and R^{e} together are propane-1,4-diyl.

The variables R^{a}′″ and R^{b}′″ in the compounds of the formulae (IIIb) together are in particular a carbonyl radical —(C═O)— or isopropylidene.

The R^{c} radical in formulae (I), (I.a), (I′), (I.a′), (III) and (IIIb) is preferably tri(C₁-C₄-alkyl)silyl, i.e. in the radical SiR^{f}R^{g}R^{h}, the radicals R^{f}, R^{g} and R^{h} are the same or different and are C₁-C₄-alkyl, or are benzyl, wherein said benzyl is unsubstituted or optionally has 1 or 2 substituents selected from fluorine, chlorine, bromine, methyl and methoxy.

The radical R^{c} in formulae (I), (I.a), (I′), (I.a′), (III) and (IIIb) is particularly preferably trimethylsilyl, benzyl, 4-chlorobenzyl, 4-methylbenzyl, 4-methoxybenzyl, 2-chlorobenzyl or 2,4-dichlorobenzyl.

The radical R^{c}′ in formulae (IIIc), (IIId), (IIId′), (IIIf), (IVb) and (IVc) is preferably benzyl, wherein said benzyl is unsubstituted or optionally has 1 or 2 substituents selected from fluorine, chlorine, bromine, methyl and methoxy.

The radical R^{c}′ in formulae (IIIc), (IIId), (IIId′), (IIIf) and (IVc) is especially benzyl, 4-chlorobenzyl, 4-methylbenzyl, 4-methoxybenzyl, 2-chlorobenzyl or 2,4-dichlorobenzyl.

The radical R^{c}″ in formula (IIIa) is preferably hydrogen or tri(C₁-C₄-alkyl)silyl, i.e. in the radical SiR^{f}R^{g}R^{h}, the radicals R^{f}, R^{g} and R^{h} are the same or different and are C₁-C₄-alkyl.

The radical R^{c}″ in formula (IIIa) is especially hydrogen or trimethylsilyl.

The radical R^{Si} is preferably tri(C₁-C₄-alkyl)silyl, particularly trimethylsilyl, i.e. in the radical SiR^{f}R^{g}R^{h}, the radicals R^{f}, R^{g} and R^{h} are the same or different and are preferably C₁-C₄-alkyl, especially methyl.

The radical R¹ in the compounds of the formulae (II) and (III) is preferably a radical C(═O)—R¹¹, where R¹¹ is hydrogen, C₁-C₄-alkyl, C₁-C₄-haloalkyl or phenyl, or the radical R¹ is a radical SiR¹²R¹³R¹⁴, wherein the radicals R¹², R¹³ and R¹⁴ are the same or different and are C₁-C₄-alkyl.

The radical R¹ in the compounds of the formulae (II) and (III) is particularly preferably trimethylsilyl or is a radical C(═O)—R¹¹, where R¹¹ is methyl, tert-butyl, phenyl or 4-chlorophenyl.

The radical R¹ in the compounds of the formulae (II) and (III) is especially trimethylsilyl, acetyl, pivaloyl, benzoyl or 4-chlorophenyl.

The radical R¹′ in the compounds of the formulae (IIIe), (IIIf), (IVb′) and (IVc) is preferably benzyl, wherein said benzyl is unsubstituted or optionally has 1 or 2 substituents selected from fluorine, chlorine, bromine, methyl and methoxy.

The radical R¹′ in the compounds of the formulae (IIIe), (IIIf), (IVb′), and (IVc) is especially benzyl, 4-chlorobenzyl, 4-methylbenzyl, 4-methoxybenzyl, 2-chlorobenzyl and 2,4-dichlorobenzyl.

The radical R¹″ in the compounds of the formulae (IIIa) and (IIIb) is preferably hydrogen, a radical C(═O)—R¹¹, where R¹¹ is hydrogen, C₁-C₄-alkyl, C₁-C₄-haloalkyl or phenyl, or is a radical SiR¹²R¹³R¹⁴, wherein the radicals R¹², R¹³ and R¹⁴ are the same or different and are C₁-C₄-alkyl.

The radical R¹″ in the compounds of the formulae (IIIa) and (IIIb) is particularly preferably hydrogen, trimethylsilyl or is a radical C(═O)—R¹¹, where R¹¹ is methyl, tert-butyl, phenyl or 4-chlorophenyl.

The radical R¹″ in the compounds of the formulae (IIIa) and (IIIb) is especially hydrogen, acetyl, pivaloyl, benzoyl or 4-chlorobenzoyl.

The radical R¹′″ in the compounds of the formulae (IVa) and (IVb) is preferably hydrogen or a radical C(═O)—R¹¹, where R¹¹ is hydrogen, C₁-C₄-alkyl, C₁-C₄-haloalkyl or phenyl.

The radical R¹′″ in the compounds of the formulae (IVa) and (IVb) is particularly preferably hydrogen or is a radical C(═O)—R¹¹, where R¹¹ is methyl, tert-butyl, phenyl or 4-chlorophenyl.

The radical R¹′″ in the compounds of the formulae (IVa) and (IVb) is especially hydrogen, acetyl, pivaloyl, benzoyl or 4-chlorobenzoyl.

In a further preferred embodiment of the present invention, in the formulae (II) and (III), the radicals
R^{a} and R^{b} are each independently —C(═O)—C₁-C₆-alkyl, —C(═O)-phenyl, wherein phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, or R^{a} and R^{b} together are a carbonyl radical —(C═O)—, and
R¹ is a radical —C(═O)—R¹¹, where R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy.

In a particularly preferred embodiment of the present invention, the radicals R^{a}, R^{b} and R¹ in formulae (II) and (III) are each independently a radical C(═O)—R¹¹, where R¹¹ is hydrogen, C₁-C₄-alkyl, C₁-C₄-haloalkyl, phenyl or 4-chlorophenyl. In certain embodiments of the invention, R¹¹ differs from methyl. In another particular embodiment of the invention, R¹¹ is methyl. In another particular embodiment of the invention, R¹¹ is tert-butyl.

In an especially preferred embodiment of the present invention, the radicals R^{a}, R^{b} and R¹ in formulae (II) and (III) are each independently acetyl, pivaloyl, benzoyl or 4-chlorobenzoyl.

With respect to the method according to the invention and the compounds of the formulae (II), (III), (IIIa), (IIIa′), (IIIb), (IIIb′), (IIIb″), (IIIb′″), (IIIc), (IIId), (IIId′), (IIIe) and (IIIf), the variables R² within one formula preferably have the same definition in each case. R² is in particular C₁-C₄-alkyl and especially methyl, or two radicals R² attached to the same carbon atom are together 1,5-pentanediyl and thus form a radical cyclohexane-1,1-diyl with the carbon atom to which they are attached. All radicals R² are especially methyl.

With respect to the method according to the invention and the compounds of the formulae (II), (III), (IIIa), (IIIa′), (IIIb), (IIIb′), (IIIb″), (IIIb′″), (IIIc), (IIId), (IIId′), (IIIe) and (IIIf), the variables R³ within one formula preferably have the same definition in each case. R³ is particularly C₁-C₄-alkyl and especially methyl.

An example of a particularly preferred compound of the formula (I) is the compound of the formula (I), wherein the radicals R^{a} and R^{b} together are isopropylidene and the radicals R^{c} and R^{Si} are both trimethylsilyl.

A further example of a particularly preferred compound of the formula (I) is the compound of the formula (I), wherein the radicals R^{a}, R^{b} and R^{c} are benzyl and R^{Si} is trimethylsilyl.

In the method according to the invention, the compound of the formula (I) is typically used in the form of the α-anomer (I-α). However, it is also possible to use the compound (I) in the form of the β-anomer (I-β) or in the form of a mixture of the α-anomer and the β-anomer. In general, the compound (I) is used in a form which largely comprises the α-anomer, i.e. the ratio of α-anomer to β-anomer is at least 9:1.

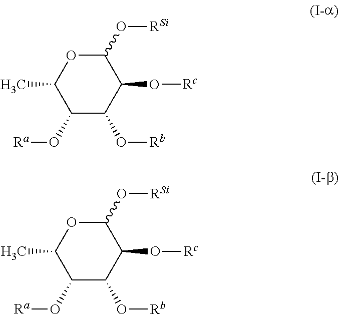

An example of a particularly preferred compound of the formula (II) is the compound of the formula (II) where all radicals R² are methyl, all radicals R³ are methyl and R¹ is trimethylsilyl.

An example of a further particularly preferred compound of the formula (II) is also the compound of the formula (II) where all radicals R² are methyl, all radicals R³ are methyl and R¹ is acetyl.

Another example of a further particularly preferred compound of the formula (II) is also the compound of the formula (II) where all radicals R² are methyl, all radicals R³ are methyl and R¹ is benzoyl.

Another example of a further particularly preferred compound of the formula (II) is also the compound of the formula (II) where all radicals R² are methyl, all radicals R³ are methyl and R¹ is pivaloyl, i.e, C(═O)—C(CH₃)₃.

Another example of a further particularly preferred compound of the formula (II) is also the compound of the formula (II) where all radicals R² are methyl, all radicals R³ are methyl and R¹ is 4-Cl-benzoyl.

Examples of especially preferred compounds of the formula (III) are
the compound of the formula (III), wherein all radicals R² are methyl, all radicals R³ are methyl, the radicals R^{a}, R^{b} and R^{c} are benzyl, 4-chlorobenzyl, 4-methylbenzyl, 2-chlorobenzyl or 2,4-dichlorobenzyl and R¹ is pivaloyl or 4-chlorobenzoyl;
the compound of the formula (III), wherein all radicals R² are methyl, all radicals R³ are methyl, the radicals R^{a} and R^{b} are benzyl, 4-chlorobenzyl, 4-methylbenzyl, 2-chlorobenzyl or 2,4-dichlorobenzyl, the radical R^{c} is trimethylsilyl and R¹ is pivaloyl or 4-chlorobenzoyl;
the compound of the formula (III), wherein all radicals R² are methyl, all radical R³ are methyl, the radicals R^{a} and R^{b} are benzoyl, 4-chlorobenzoyl, 4-methylbenzoyl or 4-fluorobenzoyl, the radical R^{c} is benzyl, 4-chlorobenzyl, 4-methylbenzyl, 2-chlorobenzyl or 2,4-dichlorobenzyl and R¹ is pivaloyl or 4-chlorobenzoyl;
the compound of the formula (III), wherein all radicals R² are methyl, all radicals R³ are methyl, the radicals R^{a} and R^{b} are benzoyl, 4-chlorobenzoyl, 4-methylbenzoyl or 4-fluorobenzyl, the radical R^{c} is trimethylsilyl and R¹ is pivaloyl or 4-chlorobenzoyl;
the compound of the formula (III), wherein all radicals R² are methyl, all radicals R³ are methyl, the radicals R^{a} and R^{b} together are isopropylidene, the radical R^{c} is trimethylsilyl and R¹ is pivaloyl or 4-chlorobenzoyl;
the compound of the formula (III), wherein all radicals R² are methyl, all radicals R³ are methyl, the radicals R^{a} and R^{b} together are isopropylidene, the radical R^{c} is benzyl, 4-chlorobenzyl, 4-methylbenzyl, 2-chlorobenzyl or 2,4-dichlorobenzyl and R¹ is pivaloyl or 4-chlorobenzoyl.

Examples of particularly preferred compounds of the formula (IIIa) are
the compound of the formula (IIIa), wherein all radicals R² are methyl, all radicals R³ are methyl, the radicals R^{a} and R^{b} together are isopropylidene and the radicals R^{c}″ and R¹″ are trimethylsilyl;
the compound of the formula (IIIa), wherein all radicals R² are methyl, all radicals R³ are methyl, the radicals R^{a} and R^{b} are benzyl, the radical R^{c}″ is hydrogen and the radical R¹ is trimethylsilyl;
the compound of the formula (IIIa), wherein all radicals R² are methyl, all radicals R³ are methyl, the radicals R^{a} and R^{b} are acetyl and the radicals R^{c}″ and R¹″ are trimethylsilyl;
the compound of the formula (III), wherein all radicals R² are methyl, all radicals R³ are methyl, the radicals R^{a} and R^{b} are acetyl, the radical R^{c}″ is hydrogen and R¹″ is trimethylsilyl;
the compound of the formula (IIIa), wherein all radicals R² are methyl, all radicals R³ are methyl, the radicals R^{a} and R^{b} together are isopropylidene, the radical R^{c}″ is trimethylsilyl and R¹″ is acetyl;
the compound of the formula (IIIa), wherein all radicals R² are methyl, all radicals R³ are methyl, the radicals R^{a} and R^{b} are benzyl, the radical R^{c}″ is hydrogen and the radical R¹″ is acetyl;
the compound of the formula (IIIa), wherein all radicals R² are methyl, all radicals R³ are methyl, the radicals R^{a}, R^{b} and R¹″ are acetyl and the radical R^{c}″ is trimethylsilyl;
the compound of the formula (IIIa), wherein all radicals R² are methyl, all radicals R³ are methyl, the radicals R^{a}, R^{b} and R¹″ are acetyl and the radical R^{c}″ is hydrogen;
the compound of the formula (IIIa), wherein all radicals R² are methyl, all radicals R³ are methyl, the radicals R^{a} and R^{b} together are isopropylidene, the radical R^{c}″ is trimethylsilyl and R¹″ is pivaloyl;
the compound of the formula (IIIa), wherein all R² radicals are methyl, all radicals R³ are methyl, the radicals R^{a} and R^{b} are benzyl, the radical R^{c}″ is hydrogen and the radical R¹′ is pivaloyl;
the compound of the formula (IIIa), wherein all radicals R² are methyl, all radicals R³ are methyl, the radicals R^{a} and R^{b} are acetyl, the radical R^{c}″ is trimethylsilyl and the radical R¹′ is pivaloyl;
the compound of the formula (IIIa), wherein all radicals R² are methyl, all radicals R³ are methyl, the radicals R^{a} and R^{b} are acetyl, the radical R^{c}″ is hydrogen and the radical R¹″ is pivaloyl;
the compound of the formula (IIIa), wherein all radicals R² are methyl, all radicals R³ are methyl, the radicals R^{a}′ and R^{b} together are isopropylidene, the radical R^{c}″ is trimethylsilyl and R¹″ is hydrogen;
the compound of the formula (IIIa), wherein all radicals R² are methyl, all radicals R³ are methyl, the radicals R^{a}′ and R^{b} are benzyl and the radicals R^{c}″ and R¹″ are hydrogen;
the compound of the formula (IIIa), wherein all radicals R² are methyl, all radicals R³ are methyl, the radicals R^{a} and R^{b} are acetyl, the radical R^{c}″ is trimethylsilyl and the radical R¹″ is hydrogen; and
the compound of the formula (IIIa), wherein all radicals R² are methyl, all radicals R³ are methyl, the radicals R^{a} and R^{b} are acetyl and the radicals R^{c}″ and R¹″ are hydrogen.

Examples of particularly preferred compounds of the formula (IIIb) are
the compound of the formula (IIIb), wherein all radicals R² are methyl, all radicals R³ are methyl, the radicals R^{a}′″ and R^{b}′″ together are isopropylidene and the radicals R^{c} and R¹″ are trimethylsilyl;
the compound of the formula (IIIb), wherein all radicals R² are methyl, all radicals R³ are methyl, the radicals R^{a}′″ and R^{b}′″ together are a carbonyl radical —(C═O)— and the radicals R^{c} and R¹″ are trimethylsilyl;
the compound of the formula (IIIb), wherein all radicals R² are methyl, all radicals R³ are methyl, the radicals R^{a}′″ and R^{b}′″ together are isopropylidene, the radical R^{c} is benzyl and R¹″ is trimethylsilyl;
the compound of the formula (IIIb), wherein all radicals R² are methyl, all radicals R³ are methyl, the radicals R^{a}′″ and R^{b}′″ together are a carbonyl radical —(C═O)—, the radical R^{c} is benzyl and R¹″ is trimethylsilyl;
the compound of the formula (IIIb), wherein all radicals R² are methyl, all radicals R³ are methyl, the radicals R^{a}′″ and R^{b}′″ together are isopropylidene, the radical R^{c} is trimethylsilyl and R¹″ is acetyl;
the compound of the formula (IIIb), wherein all radicals R² are methyl, all radicals R³ are methyl, the radicals R^{a}′″ and R^{b}′″ together are a carbonyl radical —(C═O)—, the radical R^{c} is trimethylsilyl and R¹″ is acetyl;
the compound of the formula (IIIb), wherein all radicals R² are methyl, all radicals R³ are methyl, the radicals R^{a}′″ and R^{b}′″ together are isopropylidene, the radical R^{c} is benzyl and R¹″ is acetyl;
the compound of the formula (IIIb), wherein all radicals R² are methyl, all radicals R³ are methyl, the radicals R^{a}′″ and R^{b}′″ together are a carbonyl radical —(C═O)—, the radical R^{c} is benzyl and R¹′ is acetyl;
the compound of the formula (IIIb), wherein all radicals R² are methyl, all radicals R³ are methyl, the radicals R^{a}′″ and R^{b}′″ together are isopropylidene, the radical R^{c} is trimethylsilyl and R¹″ is pivaloyl;
the compound of the formula (IIIb), wherein all radicals R² are methyl, all radicals R³ are methyl, the radicals R^{a}′″ and R^{b}′″ together are a carbonyl radical —(C═O)—, the radical R^{c} is trimethylsilyl and R¹″ is pivaloyl;
the compound of the formula (IIIb), wherein all radicals R² are methyl, all radicals R³ are methyl, the radicals R^{a}′″ and R^{b}′″ together are isopropylidene, the radical R^{c} is benzyl and R¹″ is pivaloyl;
the compound of the formula (IIIb), wherein all radicals R² are methyl, all radicals R³ are methyl, the radicals R^{a}′″ and R^{b}′″ together are a carbonyl radical —(C═O)—, the radical R^{c} is benzyl and R¹″ is pivaloyl;
the compound of the formula (IIIb), wherein all radicals R² are methyl, all radicals R³ are methyl, the radicals R^{a}′″ and R^{b}′″ together are isopropylidene, the radical R^{c} is trimethylsilyl and R¹″ is hydrogen;
the compound of the formula (IIIb), wherein all radicals R² are methyl, all radicals R³ are methyl, the radicals R^{a}′″ and R^{b}′″ together are a carbonyl radical —(C═O)—, the radical R^{c} is trimethylsilyl and R¹″ is hydrogen;
the compound of the formula (IIIb), wherein all radicals R² are methyl, all radicals R³ are methyl, the radicals R^{a}′″ and R^{b}′″ together are isopropylidene, the radical R^{c} is benzyl and R¹″ is hydrogen; and
the compound of the formula (IIIb), wherein all radicals R² are methyl, all radicals R³ are methyl, the radicals R^{a}′″ and R^{b}′″ together are a carbonyl radical —(C═O)—, the radical R^{c} is benzyl and R¹″ is hydrogen.

Examples of preferred compounds of the formula (IVa) and (IVb) are
the compound of the formula (IVa) and (IVb), where R¹¹ is methyl;
the compound of the formula (IVa) and (IVb), where R¹¹ is ethyl;
the compound of the formula (IVa) and (IVb), where R¹¹ is tert-butyl;
the compound of the formula (IVa) and (IVb), where R¹¹ is phenyl.

Examples of particularly preferred compounds of the formula (IVa) are
the compound of the formula (IVa), wherein all radicals R² are methyl, all radicals R³ are methyl, the radicals R^{a} and R^{b} together are isopropylidene and the radical R¹′″ is hydrogen;
the compound of the formula (IVa), wherein all radicals R² are methyl, all radicals R³ are methyl, the radicals R^{a} and R^{b} are benzyl and the radical R¹′″ is hydrogen;
the compound of the formula (IVa), wherein all radicals R² are methyl, all radicals R³ are methyl, the radicals R^{a} and R^{b} are acetyl and the radical R¹′″ is hydrogen;
the compound of the formula (IVa), wherein all radicals R² are methyl, all radicals R³ are methyl, the radicals R^{a} and R^{b} together are isopropylidene and the radical R¹′″ is acetyl;
the compound of the formula (IVa), wherein all radicals R² are methyl, all radicals R³ are methyl, the radicals R^{a} and R^{b} are benzyl and the radical R¹′″ is acetyl;
the compound of the formula (IVa), wherein all radicals R² are methyl, all radicals R³ are methyl, the radicals R^{a}, R^{b} and R¹′″ are acetyl;
the compound of the formula (IVa), wherein all radicals R² are methyl, all radicals R³ are methyl, the radicals R^{a} and R^{b} together are isopropylidene and the radical R¹′″ is pivaloyl;
the compound of the formula (IVa), wherein all radicals R² are methyl, all radicals R³ are methyl, the radicals R^{a} and R^{b} are benzyl and the radical R¹′″ is pivaloyl;
the compound of the formula (IVa), wherein all radicals R² are methyl, all radicals R³ are methyl, the radicals R^{a} and R^{b} are acetyl and the radical R¹′″ is pivaloyl;
the compound of the formula (IVa), wherein all radicals R² are methyl, all radicals R³ are methyl, the radicals R^{a} and R^{b} together are isopropylidene and the radical R¹′″ is benzyl;
the compound of the formula (IVa), wherein all radicals R² are methyl, all radicals R³ are methyl, the radicals R^{a}, R^{b} and R¹′″ are benzyl; and
the compound of the formula (IVa), wherein all radicals R² are methyl, all R³ radicals are methyl, the radicals R^{a} and R^{b} are acetyl and the radical R¹′″ is benzyl.

Examples of particularly preferred compounds of the formula (IVb) are
the compound of the formula (IVb), wherein all radicals R² are methyl, all radicals R³ are methyl, the radicals R^{a}′″ and R^{b}′″ together are a carbonyl radical —(C═O)—, the radical R^{c}′ is benzyl and the radical R¹″ is hydrogen;
the compound of the formula (IVb), wherein all radicals R² are methyl, all radicals R³ are methyl, the radicals R^{a}′″ and R^{b}′″ together are a carbonyl radical —(C═O)—, the radical R^{c}′ is benzyl and the radical R¹″ is acetyl;
the compound of the formula (IVb), wherein all radicals R² are methyl, all radicals R³ are methyl, the radicals R^{a}′″ and R^{b}′″ together are a carbonyl radical —(C═O)—, the radical R^{c}′ is benzyl and the radical R¹″ is pivaloyl; and
the compound of the formula (IVb), wherein all radicals R² are methyl, all radical R³ are methyl, the radicals R^{a}′″ and R^{b}′″ together are a carbonyl radical —(C═O)—, the radical R^{c}′ is benzyl and the radical R¹″ is benzyl.

Step a) of the method according to the invention comprises the treatment of the protected fucose of the general formula (I) with at least one tri(C₁-C₆-alkyl)silyl iodide. In this case, the compound of the formula (I) is selectively converted to the corresponding 1-iodofucose of the general formula (I.a):

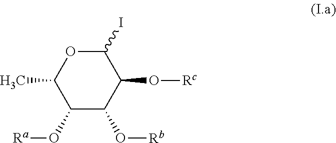

The reaction product obtained is then reacted with the compound of the formula (II), wherein the reaction takes place in the presence of at least one base, not least in order to scavenge the hydrogen iodide formed optionally in low amounts in the reaction (step b)).

The tri(C₁-C₆-alkyl)silyl iodide preferably used is trimethylsilyl iodide.

The tri(C₁-C₆-alkyl)silyl iodide is preferably used in an amount of 0.8 mol to 1.4 mol or 0.8 mol to 1.2 mol, particularly in an amount of 0.9 to 1.1 mol, especially in an amount of 0.9 to 1 mol per mole of the compound of the formula (I).

The tri(C₁-C₆-alkyl)silyl iodide, particularly trimethylsilyl iodide, can be used as such, Tri(C₁-C₆-alkyl)silyi iodide, particularly trimethylsilyl iodide, may also be prepared in situ.

The in situ preparation of tri(C₁-C₆-alkyl)silyl iodide succeeds by way of example by treatment of the corresponding tri(C₁-C₆-alkyl)silyl chloride with an iodide salt, particularly an alkali metal iodide, such as lithium iodide, potassium iodide or sodium iodide. Methods for this purpose are known, e.g. from Synthesis 1983, p. 459, Synthesis 1979, p. 740, Synthesis 1981, p. 67, Chem. Ber, 1962, 95 p. 174 and Bioorganic and Med. Chem. Lett. 10, 2000, p 2311. For this purpose, the iodide salt is preferably used in at least an equimolar amount, based on the tri(C₁-C₆-alkyl)silyl chloride, particularly in excess, based on the tri(C₁-C₆-alkyl)silyl chloride. In this case, the preferred procedure is such that the tri(C₁-C₆-alkyl)silyl iodide, particularly trimethylsilyl iodide, is initially prepared by treatment of the corresponding tri(C₁-C₆-alkyl)silyl chloride with an iodide salt, particularly with an alkali metal iodide, such as lithium iodide, potassium iodide or sodium iodide and the reaction product is added to the compound of the general formula (I). The preparation is preferably carried out in a suitable solvent, particularly in an aprotic solvent, such as acetonitrile or propionitrile.

The in situ preparation of the tri(C₁-C₆-alkyl)silyl iodide succeeds for example by treatment of the corresponding hexa(C₁-C₆-alkyl)disilane, particularly the hexamethyldisilane (HMDS) with iodine. Methods for this purpose are known, e.g. from Synthesis Commun. 1974, p. 740; Chem. Commun, 2003, p. 1266; Carb, Lett. 1998, 3, p. 179.

In this regard, the procedure preferably involves reacting with one another the hexa(C₁-C₆-alkyl)disilane, particularly HMDS, with elemental iodine in an upstream reaction step and adding the reaction mixture thus obtained to compound (I). The hexa(C₁-C₆-alkyl) disilane, particularly HMDS, can be reacted with iodine without solvent or in an inert organic solvent. Suitable solvents are especially halohydrocarbons such as chloroform and dichloromethane. The reaction of hexa(C₁-C₆-alkyl)disilane, particularly HMDS, is generally carried out with elemental iodine at temperatures in the range of 0 to 110° C., especially in the range of 0 to 60° C. Alternatively, the hexa(C₁-C₆-alkyl)disilane, particularly HMDS, can be reacted with iodine and compound (I). This variant is likewise preferably carried out in an inert organic solvent. Suitable solvents here are also especially halohydrocarbons such as chloroform and dichloromethane. Preference is given to using hexa(C₁-C₆-alkyl)disilane and iodine in a molar ratio in the range from 0.5:1 to 1:0.5, especially in a molar ratio of approximately 1:1. Preference is given to using hexa(C₁-C₆-alkyl) disilane and compound (I) in a molar ratio in the range from 0.5:1 to 1:1, particularly in the range from 0.5:1 to 0.8:1. Preference is given to using iodine and compound (I) in a molar ratio in the range from 0.5:1 to 1:1, particularly in the range from 0.5:1 to 0.8:1.

The compound of the formula (I) is generally reacted with the tri(C₁-C₆-alkyl)silyl iodide in an inert organic solvent or diluent. Preference is given to aprotic solvents, particularly those having a low content of protic impurities such as water, alcohols or acid. The content of protic impurities in the solvent is preferably less than 1000 ppm. Preferably before use in the method according to the invention, the aprotic solvent is treated to reduce the content of protic impurities, particularly water, by treatment with suitable absorbents, for example with molecular sieves of pore size 3 to 4 Angström. Preferred organic solvents are alkenes and cycloalkenes such as isobutene, amylene (1-pentene, 2-pentene, 2-methylbut-1-ene, 2-methylbut-2-ene, 3-methylbut-1-ene and mixtures thereof), cyclopentene and cyclohexene, haloalkanes such as dichloromethane, trichloromethane, dichloroethane, aromatic hydrocarbons such as toluene and xylenes and also alkylnitriles such as acetonitrile, and also mixtures of the aforementioned solvents. The solvent is preferably selected such that all constituents are present in dissolved form. The total concentration of compound of the formula (I) is preferably in the range of 5 to 70% by weight, particularly 10 to 50% by weight, based on the total weight of all reagents and solvents. For example, the method may be carried out in an aprotic solvent different from alkenes with addition of 5 to 100 mol %, based on compound (I), of at least one alkene or the reaction may be carried out also in this alkene as solvent or the alkene may be added for stabilization at the end of the reaction. In this case, the alkene serves to capture I₂ or HI.

The compound of the formula (I) is reacted with the tri(C₁-C₆-alkyl)silyl iodide preferably at temperatures in the range from −20 to 110° C., particularly in the range from 0 to 80° C. and especially in the range from 20 to 65° C. The reaction may be carried out at ambient pressure, at reduced or elevated pressure. The reaction is typically conducted at a pressure in the range of 900 to 1100 mbar.

The reaction product resulting from the reaction of the compound of the formula (I) with the tri(C₁-C₆-alkyl)silyl iodide is preferably not isolated, but is reacted without further isolation or purification with the compound of the formula (II), particularly in the presence of the base, wherein the compound of the formula (III) is obtained. The reaction product of the general formula (I.a) resulting from the reaction of the compound of the formula (I) with the tri(C₁-C₆-alkyl)silyl iodide can also be purified or isolated, for example by removing volatile constituents from the reaction mixture, preferably under reduced pressure and/or by co-evaporation with suitable low-boilers, e.g. alkanes such as hexane, cyclohexane or heptane, or aromatic compounds such as toluene.

Optionally, an inorganic base from the group of the alkali metal carbonates and alkaline earth metal carbonates and also alkali metal hydrogen carbonates and alkaline earth metal hydrogen carbonates, particularly an inorganic base from the group of the alkali metal carbonates such as lithium, sodium or potassium carbonate and alkali metal hydrogen carbonates such as sodium hydrogen carbonate and potassium hydrogen carbonate, can be added to the 1-iodofucose (I.a) obtained in step a) prior to the reaction with compound (II) in step b). If desired, this inorganic base is added in an amount of in particular 0.01 to 0.5 equivalents per mole of the compound of the formula (I.a), i.e. in the case of a carbonate in an amount of 0.005 to 0.25 mol per mole of compound (I.a) and in the case of a hydrogen carbonate in an amount of 0.01 to 0.5 mol per mole of compound (I.a).

The 1-iodofucose (I.a) obtained in step a), i.e. from the treatment of compound (I) with the tri(C₁-C₆-alkyl)silyl iodide, is reacted in accordance with the invention with the compound of the formula (II) in step b).

The reaction in step b) takes place in the presence of at least one base. In order to avoid secondary reactions, the base is preferably used in at least an equimolar amount, based on the compound of the formula (I.a). In particular, the base is used in an amount of 1 to 3 mol per mole of the compound of the formula (I.a), particularly in an amount of 1 to 1.5 mol per mole of the compound of the formula (I.a).

Preferred bases are primarily amine bases, particularly secondary and tertiary amines, especially pyridine bases and also tertiary aliphatic or cycloaliphatic amines. Suitable pyridine bases are, for example, pyridine, quinoline and C₁-C₆-alkyl-substituted pyridines, particularly mono-, di- and tri(C₁-C₆-alkyl)pyridines such as 2,6-di(C₁-C₆-alkyl) pyridines, e.g. 2,6-dimethylpyridine or 2,6-bis(tert-butyl)pyridine, and collidine. Suitable tertiary aliphatic or cycloaliphatic amines are tri(C₁-C₆-alkyl)amines such as trimethylamine, triethylamine, diisopropylmethylamine, tri-n-butylamine or isopropyldimethylamine, C₃-C₈-cycloalkyl-di(C₁-C₆-alkyl)amines such as cyclohexyldimethylamine, N—(C₁-C₆-alkyl)piperidine such as N-methylpiperidine and di(C₃-C₈-cycloalkyl)-C₁-C₆-alkylamines such as biscyclohexylmethylamine. Particular preference is given to tri(C₁-C₆-alkyl)amines, especially trimethylamine and triethylamine. Suitable bases are also inorganic bases from the group of the alkali metal carbonates and alkaline earth metal carbonates and also alkali metal hydrogen carbonates and alkaline earth metal hydrogen carbonates, particularly inorganic bases from the group of the alkali metal carbonates such as lithium, sodium or potassium carbonate, and alkali metal hydrogen carbonates such as sodium hydrogen carbonate and potassium hydrogen carbonate.

The base preferably comprises at least one amine base, in particular at least one tertiary amine. The base particularly preferably comprises at least one amine base, in particular at least one tertiary amine and at least one further inorganic base selected from alkali metal carbonates and alkali metal hydrogen carbonates. If a combination of amine base and alkali metal carbonate or hydrogen carbonate is used, the amine base is preferably used in an amount of 1 to 2 mol per mole of the compound of the formula (I.a), particularly in an amount of 1 to 1.5 mol per mole of the compound of the formula (I.a). If desired, the inorganic base is used in an amount of in particular 0.01 to 0.5 equivalents per mole of the compound of the formula (I.a), i.e. in the case of a carbonate in an amount of 0.005 to 0.25 mol per mole of compound (I.a) and in the case of a hydrogen carbonate in an amount of 0.01 to 0.5 mol per mole of compound (I.a).

The compound of the formula (II) is generally used in such an amount that the molar ratio of compound of the formula (I.a) to the compound of the formula (II) is in the range from 1:3 to 3:1, particularly in the range from 1:2 to 2:1, particularly preferably in the range from 1:1.5 to 1.5:1, and especially in the range from 1:1,1 to 1.1:1.

Step b) is preferably carried out in the presence of at least one reagent selected from iodine, iodide salts and triarylphosphine oxides and mixtures thereof. Suitable iodide salts, in addition to alkali metal iodides, are primarily tetraalkylammonium iodides, particularly tetra-C₁-C₆-alkylammonium iodide, such as tetraethylammonium iodide, tetrapropylammonium iodide and especially tetrabutylammonium iodide. Preference is given to alkali metal iodides and especially NaI and KI. A suitable triarylphosphine oxide is particularly triphenylphosphine oxide. In particular, step b) is carried out in the presence of at least one reagent selected from iodine and iodide salts, particularly from iodine and alkali metal iodides and mixtures thereof. Specifically, step b) is carried out in the presence of a mixture of iodine and iodide salts, in particular a mixture of iodine and alkali metal iodides and more specifically in the presence of a mixture of iodine and KI or a mixture of iodine and NaI.

In a first preferred embodiment A of the invention, the reaction in step b) takes place in the presence of iodine. In this embodiment, the tri(C₁-C₆-alkyl)silyl iodide is preferably used in an amount of 0.9 to 1.1 mol, especially in an amount of 0.9 to 1 mol, per mole of the compound of the formula (I), and iodine is preferably used in an amount of 0.005 to 0.5 mol, particularly in an amount of 0.005 to 0.1 mol per mole of the compound of the formula (I.a).

In a further preferred embodiment B of the invention, the reaction in step b) takes place in the presence of an iodide salt. In this embodiment, the tri(C₁-C₆-alkyl)silyl iodide is preferably used in an amount of 0.9 to 1.1 mol, especially in an amount of 0.9 to 1 mol, per mole of the compound of the formula (I), and the iodide salt is preferably used in an amount of 0.005 to 0.5 mol, particularly in an amount of 0.01 to 0.1 mol per mole of the compound of the formula (I.a). Suitable iodide salts, in addition to alkali metal iodides, are primarily tetraalkylammonium iodides, particularly tetra-C₁-C₆-alkylammonium iodide, such as tetraethylammonium iodide, tetrapropylammonium iodide and especially tetrabutylammonium iodide. Preference is given to alkali metal iodides and especially NaI and KI.

In a further preferred embodiment C of the invention, the reaction in step b) takes place in the presence of iodine and an iodide salt. In this embodiment, the tri(C₁-C₆-alkyl)silyl iodide is preferably used in an amount of 0.9 to 1.1 mol, especially in an amount of 0.9 to 1 mol, per mole of the compound of the formula (I), and the iodine and iodide salt is preferably used in an amount of 0.005 to 0.5 mol, particularly in an amount of 0.005 to 0.1 mol per mole of the compound of the formula (I.a). Suitable iodide salts, in addition to alkali metal iodides such as KI and NaI, are primarily tetraalkylammonium iodides, particularly tetra-C₁-C₆-alkylammonium iodide, such as tetraethylammonium iodide, tetrapropylammonium iodide and especially tetrabutylammonium iodide. Preference is given to alkali metal iodides and especially NaI and KI.

In a further preferred embodiment D of the invention, the reaction in step b) takes place in the presence of a triarylphosphine oxide. In this embodiment, the tri(C₁-C₆-alkyl)silyl iodide is preferably used in an amount of 0.9 to 1.1 mol, especially in an amount of 0.9 to 1 mol, per mole of the compound of the formula (I), and the triarylphosphine oxide is preferably used in an amount of 0.005 to 0.5 mol and especially in an amount of 0.005 to 0.1 mol per mole of the compound of the formula (I.a). A suitable triarylphosphine oxide is particularly triphenylphosphine oxide.

In an equally preferred embodiment of the invention, none of the abovementioned reagents is added in step b).

In a particularly preferred embodiment, the method proceeds in the following manner. Firstly, the hexaalkyl(C₁-C₆-alkyl)disilane is reacted with iodine in step a), and the reaction mixture thus obtained is subsequently reacted with the compound of the formula (I). The reaction is effected in general under the conditions stated above, particularly under the conditions specified as preferred. To the resulting reaction mixture is then added at least one inorganic base, selected from alkali metal carbonates, alkali metal hydrogen carbonates and mixtures thereof, and the mixture thus obtained is reacted with the compound of the general formula (II) in the presence of the amine base (step b)). With regard to the reaction conditions, amounts of base and reagents, that which is stated above likewise applies. In this embodiment, step b) is carried out in the presence of at least one reagent selected from iodine and iodide salts, particularly from iodine and alkali metal iodides and mixtures thereof. Specifically, step b) of this embodiment is carried out in the presence of a mixture of iodine and iodide salts, in particular a mixture of iodine and alkali metal iodides and more specifically in the presence of a mixture of I and KI or a mixture of iodine and NaI. With regard to the quantitative ratios of these reagents, that which has been stated above for embodiments A, B and C applies analogously.

Step b), i.e. the reaction of the reaction product resulting from treatment of the compound of the formula (I) with the tri(C₁-C₆-alkyl)silyl iodide, i.e. the 1-iodofucose (I.a), with the compound of the formula (II), is generally carried out in one of the abovementioned inert organic solvents or diluents. Preference is also given here to the abovementioned aprotic solvents, particularly those having a low content of protic impurities such as water, alcohols or acid. The content of protic impurities in the solvent is preferably less than 1000 ppm. Preferably before use in the method according to the invention, the aprotic solvent is treated to reduce the content of protic impurities, particularly water, by treatment with suitable absorbents, for example, with molecular sieves of pore size 3 to 4 Angström. Preferred organic solvents are haloalkanes such as dichloromethane, trichloromethane, dichloroethane, aromatic hydrocarbons such as toluene and xylenes, dimethylamides of aliphatic carboxylic acids such as dimethylformamide (DMF) or dimethylacetamide, and also alkylnitriles such as acetonitrile, and also mixtures of the abovementioned solvents. The solvent is preferably selected such that all constituents are present in dissolved form. The total concentration of compound of the formula (I.a) and (II) is preferably in the range of 5 to 75% by weight, particularly 10 to 65% by weight or 15 to 60% by weight, based on the total weight of all reagents and solvents.

The reaction in step b) is preferably carried out at temperatures in the range of −20 to 110° C., particularly in the range of 0 to 80° C. The reaction may be carried out at ambient pressure, at reduced or elevated pressure. The reaction is typically conducted at a pressure in the range of 900 to 1100 mbar.

The compound of the formula (III) obtained by the reaction in step b) may be isolated by customary work-up methods and optionally be purified by crystallization and/or chromatography. Alternatively, it is possible to directly subject the compound of the formula (III) obtained by the reaction in step b) to at least partial deprotection so as thus to obtain the compounds of the formulae (IIIa), (IIIb) or the compound of the formulae (IVa) or (IVb).

The deprotection of the compound of the formula (III) is achieved in analogy to known deprotecting reactions and is preferably carried out by hydrolysis methods. The conditions for cleavage of protecting groups are familiar to those skilled in the art, e.g. from P. G. M. Wuts et al., “Greene's Protecting Groups in Organic Synthesis”, 4th Edition, Wiley 2006 and the literature cited therein, or the literature cited at the outset for preparing 2′-O-fucosyllactose.

According to a first preferred embodiment c.1) of the invention, the compound of the formula (III), in which
R^{a} and R^{b} are the same or different and are —C(═O)—C₁-C₆-alkyl, —C(═O)-phenyl, wherein phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, or R^{a} and R^{b} together are a carbonyl radical —(C═O)—,
R^{c} is a radical R^{Si},
R¹ is a radical —C(═O)—R¹¹ or a radical SiR¹²R¹³R¹⁴, in which
   R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
   R¹², R¹³ and R¹⁴ are the same or different and are selected from C₁-C₈-alkyl, C₃-C₈-cycloalkyl, phenyl and C₃-C₈-cycloalkyl-C₁-C₄-alkyl,
R² and R³ have the definitions stated above,

is firstly treated with a C₁-C₄-alkanol and an alkali metal base, wherein a compound of the formula (IIIb″) is obtained:

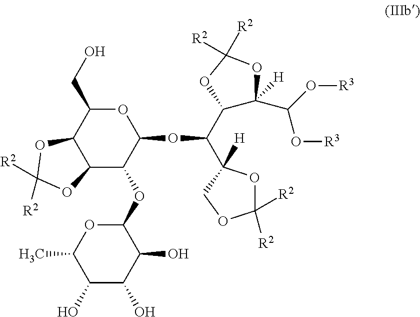

in which
R² and R³ have the definitions stated above,

and subsequently the remaining protecting groups are removed by treating the compound of the formula (IIIb′) with water in the presence of an acid. In this manner, a complete cleavage of all protecting groups from the compound of the formula (III) is generally achieved and the 2′-O-fucosyllactose is obtained.

In this embodiment, R¹¹ is preferably C₁-C₄-alkyl such as methyl, ethyl or tert-butyl. By treating the compounds (III) mentioned under embodiment c.1) with a C₁-C₄-alkanol and an alkali metal base, the desilylation and the removal of the ester groups can be combined with one another and cleaved in one step. Suitable reagents are alkali metal hydroxides and carbonates, such as lithium hydroxide, potassium hydroxide, sodium hydroxide, lithium carbonate, sodium carbonate or potassium carbonate, in C₁-C₄-alkanols such as methanol, ethanol, isopropanol, 1-butanol or tert-butanol, particularly methanol. Particularly suitable is the combination of methanol with sodium carbonate or potassium carbonate. The reaction conditions required for this purpose are familiar to those skilled in the art and may be determined by routine experiments. The simultaneous desilylation and removal of the C(═O)—R¹¹ ester group is achieved by treatment with an alkali metal base in C₁-C₄-alkanols such as methanol at temperatures in the range of 20 to 50° C. The amount of alkali metal base, particularly alkali metal carbonate, is preferably 3 to 10 equivalents and in particular 4 to 7 equivalents, based on the compound (III), or in the case of an alkali metal carbonate 1.5 to 5 mol, in particular 2 to 3.5 mol per mole of compound (III).

Alternatively, the desilylation and the removal of the ester groups can also be carried out stepwise:

The desilylation of the compounds of the formula (III) mentioned under embodiment c.1) is possible by treating compound (III) with a desilylating reagent. Suitable reagents for the desilylation are, for example, the abovementioned C₁-C₄ alcohols, particularly methanol, with or without addition of water, and also alkali metal or alkaline earth metal carbonates and hydrogen carbonates, such as lithium carbonate, sodium carbonate, potassium carbonate, sodium hydrogen carbonate and potassium hydrogen carbonate, preferably in solution in one of the abovementioned C₁-C₄ alcohols, particularly methanol, with or without addition of water. Suitable desilylating reagents are also tetraalkylammonium fluorides, which are preferably used in polar, aprotic organic solvents, e.g. cyclic ethers such as tetrahydrofuran or dioxane, or in di-C₁-C₄-alkylamides of aliphatic carboxylic acids such as dimethylformamide or dimethylacetamide, or alkyl nitriles such as acetonitrile or mixtures of the abovementioned polar, aprotic organic solvents. The reaction conditions required are known to a person skilled in the art, e.g. from P. G. M. Wuts et al., loc. cit. and the literature cited therein.

The subsequent cleavage of the ester groups is achieved in a manner known per se by basic saponification or by base-catalyzed or enzyme-catalyzed transesterification. Methods for this purpose are known, e.g. from P. G. M. Wuts et al. loc. cit. or from Kociensky et al. Protective groups, 3rd edition, Chapter 4.6, Thieme 2005.

The C(R²)₂ and OR³ protecting groups are cleaved with water in the presence of an acid. Suitable acids are mineral acids, such as hydrochloric acid, sulfuric acid, phosphoric acid, acidic salts of mineral acids such as alkali metal hydrogen phosphates and dihydrogen phosphates or alkali metal hydrogen sulfates, e.g. sodium dihydrogen phosphate or potassium hydrogen phosphate, in addition organic carboxylic acids, such as acetic acid, propionic acid, dichloroacetic acid, trichloroacetic acid or trifluoroacetic acid, and organic sulfonic acids, such as methanesulfonic acid. The acids are typically used as dilute aqueous acids, e.g. as 5 to 70% strength by weight solutions. Frequently, the dilute aqueous acid is used in combination with a suitable organic solvent. Examples thereof are organic solvents miscible with water, such as C₁-C₄-alkanols, e.g. methanol, ethanol, isopropanol, 1-butanol or tert-butanol, cyclic ethers such as tetrahydrofuran or dioxane, and also organic solvents having only limited miscibility with water, e.g. haloalkanes such as dichloromethane, trichloromethane, dichloroethane, aromatic hydrocarbons such as toluene and xylenes, and also dialkyl ethers such as diethyl ether, diisopropyl ether or methyl tert-butyl ether. The reaction conditions required are known to a person skilled in the art, e.g. from P. G. M. Wuts et al., loc. cit. and the literature cited therein, or the references cited at the outset for the preparation of 2′-O-fucosyllactose. After cleavage of the protecting groups, typically the acid is neutralized and then the product is isolated by removing the water. For the neutralization, the bases normally used for this purpose can be used, e.g. alkali metal hydroxides, carbonates and hydrogencarbonates. The neutralization can also be carried out, for example, using basic or strongly basic ion exchangers, since in this case the neutralization can be effected without releasing salts into the solution.

The cleavage of the C(R²)₂ and OR³ protecting groups according to embodiment c.1) may also be carried out by means of aqueous acidic ion exchange. In this manner, a separate neutralization can be avoided.

According to a further embodiment c.2) of the invention, the compound of the formula (III), in which
R^{a} and R^{b} together are a substituted methylene radical —C(R^{d}R^{e})—, where R^{d} and R^{e} are the same or different and are selected from hydrogen, phenyl and C₁-C₄-alkyl or both radicals R^{d} and R^{e} together are linear C₄-C₆-alkenyl,
R^{c} is a radical R^{Si},
R¹ is a radical —C(═O)—R¹¹ or a radical SiR¹²R¹³R¹⁴, in which
   R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
   R¹², R¹³ and R¹⁴ are the same or different and are selected from C₁-C₈-alkyl, C₃-C₈-cycloalkyl, phenyl and C₃-C₈-cycloalkyl-C₁-C₄-alkyl, and
R² and R³ have the definitions stated above,

is firstly treated with a C₁-C₄-alkanol and an alkali metal base, wherein a compound of the formula (IIIb″) is obtained:

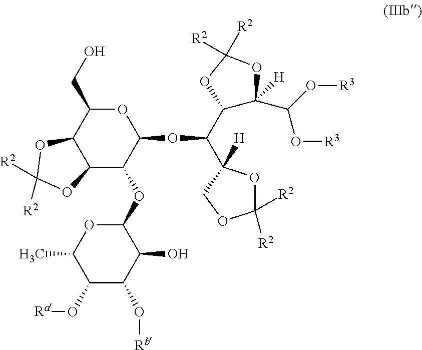

in which
R^{a}′ and R^{b}′ together are a substituted methylene radical —C(R^{d}R^{e})—, where R^{d} and R^{e} are the same or different and are selected from hydrogen, phenyl and C₁-C₄-alkyl or both radicals R^{d} and R^{e} together are linear C₄-C₆-alkenyl, and
R² and R³ have the definitions stated above,

and subsequently the remaining protecting groups are removed by treating the compound of the formula (IIIb′) with water in the presence of an acid.

The treatment of the compounds of the formula (III) mentioned under c.2) with C₁-C₄-alkanol and an alkali metal base and the treatment of the compound of the formula) (IIIb′) with water in the presence of an acid can be carried out in the manner described for embodiment c.1). Optionally, the desilylation and the removal of the ester groups can also be carried out stepwise as described for embodiment c.1).

According to a further embodiment c.3) of the invention, the compound of the formula (III), in which
R^{a} and R^{b} are the same or different and are benzyl, wherein said benzyl is unsubstituted or optionally has 1, 2 or 3 substituents selected from halogen, C₁-C₄-alkyl or C₁-C₄-alkoxy,
R^{c} is a radical R^{Si},
R¹ is a radical —C(═O)—R¹¹ or a radical SiR¹²R¹³R¹⁴, in which
   R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
   R¹², R¹³ and R¹⁴ are the same or different and are selected from C₁-C₈-alkyl, C₃-C₈-cycloalkyl, phenyl and C₃-C₈-cycloalkyl-C₁-C₄-alkyl, and
R² and R³ have the definitions stated above,

is firstly treated with a C₁-C₄-alkanol and an alkali metal base, wherein a compound of the formula (IIIb″) is obtained:

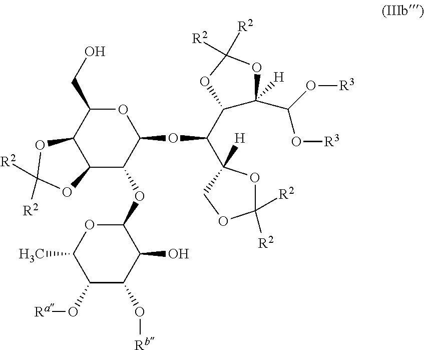

in which
R^{a}″ and R^{b}″ are the same or different and are benzyl, wherein said benzyl is unsubstituted or optionally has 1, 2 or 3 substituents selected from halogen, C₁-C₄-alkyl or C₁-C₄-alkoxy, and
R² and R³ have the definitions stated above,

subsequently the compound of the formula (IIIb′″) is treated with water in the presence of an acid, wherein a compound of the formula (IVa′) is obtained:

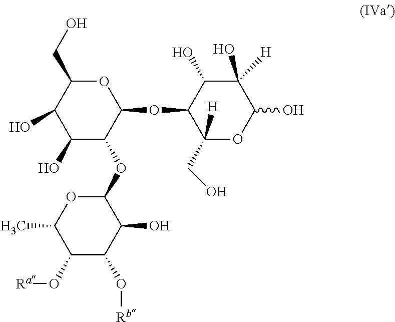

in which
R^{a}″ and R^{b}″ are the same or different and are benzyl, wherein said benzyl is unsubstituted or optionally has 1, 2 or 3 substituents selected from halogen, C₁-C₄-alkyl or C₁-C₄-alkoxy,

and subsequently the remaining benzylic protective groups are removed with hydrogen in the presence of a hydrogenation catalyst or oxidatively.

The treatment of the compounds of the formula (III) mentioned under c.3) with C₁-C₄-alkanol and an alkali metal base and the treatment of the compound of the formula (IIIb′″) with water in the presence of an acid can be carried out in the manner described for embodiment c.1). Optionally, the desilylation and the removal of the ester groups can also be carried out stepwise as described for embodiment c,1).

The removal of the remaining benzylic protecting groups in the compounds of the formula (IVa′) is achieved in a manner known per se either with hydrogen in the presence of a hydrogenation catalyst or by treating the compound of the formula (IVa′) with an oxidizing agent and a base. Methods for this purpose are known, e.g. from P. G. M. Wuts et al. and the documents cited therein or the references cited at the outset for preparing 2′-O-fucosyllactose.

The removal of the benzylic protecting groups with hydrogen in the presence of a hydrogenation catalyst may be carried out as described in WO 2010/115935. Accordingly, the removal of the benzylic protecting groups with hydrogen in the presence of a hydrogenation catalyst is typically carried out in a protic solvent or in a mixture of protic solvents. For this purpose, suitable protic solvents are typically selected from water, acetic acid or C₁-C₆-alcohols. Mixtures of one or more protic solvents with one or more aprotic solvents which are partially or completely miscible with the protic solvent(s), such as THF, dioxane, ethyl acetate, acetone or the like, can also be used. The solvent used is preferably water, one or more C₁-C₆-alcohols or a mixture of water with one or more C₁-C₆-alcohols. Solutions are likewise suitable which comprise the carbohydrate derivatives in any concentration and also suspensions of the carbohydrate derivatives in the solvent(s) mentioned. The reaction mixture is generally stirred at a temperature in the range from 10 to 100° C., preferably in the range from 20 to 50° C., and a hydrogen pressure in the range from 1 to 50 bar in the presence of the hydrogenation catalyst, such as palladium, Raney nickel or another suitable metal catalyst, preferably palladium on carbon or palladium black, until completion of the reaction. The concentration of the hydrogenation catalyst in the reaction mixture is generally in the range from 0.1% to 10% by weight, preferably in the range from 0.15% to 5% by weight, especially in the range from 0.25% to 2.25% by weight, based on the weight of the benzyl-protected carbohydrate compound used. A transfer hydrogenation can also be carried out by generating hydrogen in situ from cyclohexene, cyclohexadiene, formic acid or ammonium formate. The hydrogenolysis is preferably carried out in a neutral pH range, for example in a pH range from 6.5 to 7.5. However, organic or inorganic bases or acids and/or basic or acidic ion exchange resins may also be added to the catalytic hydrogenolysis in order to improve the kinetics of the hydrogenolysis. The use of basic additives is then particularly advantageous for example if the benzyl protecting groups are halogen-substituted benzyl groups. Organic acids are preferred as co-solvent or additive, for example in the cases when two or more benzyl groups have to be removed. Suitable organic bases which may be used as additive in the catalytic hydrogenolysis are, for example, triethylamine, diisopropylethylamine, ammonia, ammonium carbamate, diethylamine and the like. Suitable organic acids which may be used as additive in the catalytic hydrogenolysis are, for example, formic acid, acetic acid, propionic acid, chloroacetic acid, dichloroacetic acid, trifluoroacetic acid and the like.

With the hydrogenolysis conditions specified, complete cleavage of the benzylic protecting groups from the compound of the formula (III) can generally be achieved, wherein 2′-O-fucosyllactose is obtained in excellent yield and high purity.

Alternatively, the benzylic protecting groups may be removed oxidatively. In this case, this takes the form of methods generally known to those skilled in the art. In the oxidative removal of the benzyl protecting groups, the benzyl-protected starting compound is initially treated with a suitable oxidizing agent, wherein the benzylic methylene group is oxidized to a carbonyl group. Suitable oxidizing agents are, for example, ozone or ruthenium(VIII) oxide, especially ozone. The benzoyl groups thus obtained can then be saponified with base, as in the manner described for embodiment c.1).

According to a further embodiment c.4) of the invention, the compound of the formula (III), in which
R^{a} and R^{b} are the same or different and are benzyl, wherein said benzyl is unsubstituted or optionally has 1, 2 or 3 substituents selected from halogen, C₁-C₄-alkyl or C₁-C₄-alkoxy,
R^{c} is benzyl, wherein said benzyl is unsubstituted or optionally has 1, 2 or 3 substituents selected from halogen, C₁-C₄-alkyl or C₁-C₄-alkoxy,
R¹ is a radical —C(═O)—R¹¹ or a radical SiR¹²R¹³R¹⁴, in which
   R¹¹ is hydrogen, C₁-C₈-alkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
   R¹², R¹³ and R¹⁴ are the same or different and are selected from C₁-C₈-alkyl, C₃-C₈-cycloalkyl, phenyl and C₃-C₈-cycloalkyl-C₁-C₄-alkyl, and
R² and R³ have the definitions stated above,

is firstly treated with a C₁-C₄-alkanol and an alkali metal base, wherein a compound of the formula (IIIc) is obtained:

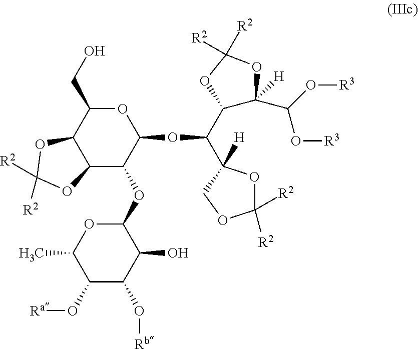

in which
R^{a}″ , R^{b}″ and R²′ are each independently benzyl, wherein said benzyl is unsubstituted or optionally has 1, 2 or 3 substituents selected from halogen, C₁-C₄-alkyl or C₁-C₄-alkoxy, and
R² and R³ have the definitions stated above,

subsequently the compound of the formula (IIIc) is treated with water in the presence of an acid, and subsequently the remaining benzylic protective groups are removed with hydrogen in the presence of a hydrogenation catalyst or oxidatively.

The treatment of the compounds of the formula (III) mentioned under c.4) with C₁-C₄-alkanol and an alkali metal base and the treatment of the compound of the formula (IIIc) with water in the presence of an acid can be carried out in the manner described for embodiment c.1). If R¹ in the compounds of the formula (III) mentioned under c.4) is a silyl protecting group, the desilylation can also be effected using the desilylating reagents in the manner described above. The benzylic protecting groups can be removed in the manner described for embodiment c.3).

According to a further embodiment c.5) of the invention, the compound of the formula (III), in which
R^{a} and R^{b} are the same or different and are —C(═O)—C₁-C₆-alkyl, —C(═O)-phenyl, wherein phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, or R^{a} and R^{b} together are a carbonyl radical —(C═O)—,
R^{c} is benzyl, wherein said benzyl is unsubstituted or optionally has 1, 2 or 3 substituents selected from halogen, C₁-C₄-alkyl or C₁-C₄-alkoxy,
R¹ is a radical —C(═O)—R¹¹ or a radical SiR¹²R¹³R¹⁴, in which
   R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
   R¹², R¹³ and R¹⁴ are the same or different and are selected from C₁-C₈-alkyl, C₃-C₈-cycloalkyl, phenyl and C₃-C₈-cycloalkyl-C₁-C₄-alkyl, and
R² and R³ have the definitions stated above,

is firstly treated with a C₁-C₄-alkanol and an alkali metal base, wherein a compound of the formula (IIId) is obtained:

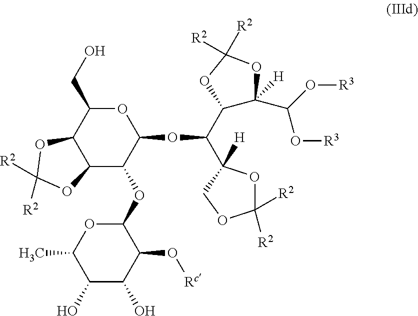

in which
R^{c}′ is benzyl, wherein said benzyl is unsubstituted or optionally has 1, 2 or 3 substituents selected from halogen, C₁-C₄-alkyl or C₁-C₄-alkoxy, and
R² and R³ have the definitions stated above,

subsequently the compound of the formula (IIId) is treated with water in the presence of an acid, and the remaining benzylic protective groups are removed with hydrogen in the presence of a hydrogenation catalyst or oxidatively.

The treatment of the compounds of the formula (III) mentioned under c.5) with C₁-C₄-alkanol and an alkali metal base and the treatment of the compound of the formula (IIId) with water in the presence of an acid can be carried out in the manner described for embodiment c.1). If R¹ in the compounds of the formula (III) mentioned under c.5) is a silyl protecting group, the desilylation can also be effected using the desilylating reagents in the manner described above. The benzylic protecting group can be removed in the manner described for embodiment c.3).

According to a further embodiment c.6) of the invention, the compound of the formula (III), in which
R^{a} and R^{b} together are a substituted methylene radical —C(R^{d}R^{e})—, where R^{d} and R^{e} are the same or different and are selected from hydrogen, phenyl and C₁-C₄-alkyl or both radicals R^{d} and R^{e} together are linear C₄-C₆-alkenyl,
R^{c} is benzyl, wherein said benzyl is unsubstituted or optionally has 1, 2 or 3 substituents selected from halogen, C₁-C₄-alkyl or C₁-C₄-alkoxy,
R¹ is a radical —C(═O)—R¹¹ or a radical SiR¹²R¹³R¹⁴, in which
   R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
   R¹², R¹³ and R¹⁴ are the same or different and are selected from C₁-C₈-alkyl, C₃-C₈-cycloalkyl, phenyl and C₃-C₈-cycloalkyl-C₁-C₄-alkyl, and
R² and R³ have the definitions stated above,

is firstly treated with a C₁-C₄-alkanol and an alkali metal base, wherein a compound of the formula (IIId′) is obtained:

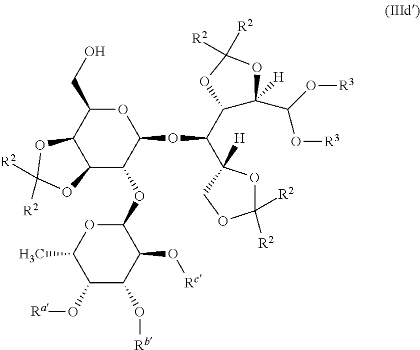

in which
R^{a}′ and R^{b}′ together are a substituted methylene radical —C(R^{d}R^{e})—, where R^{d} and R^{e} are the same or different and are selected from hydrogen, phenyl and C₁-C₄-alkyl or both radicals R^{d} and R^{e} together are linear C₄-C₆-alkenyl,
R^{c}′ is benzyl, wherein said benzyl is unsubstituted or optionally has 1, 2 or 3 substituents selected from halogen, C₁-C₄-alkyl or C₁-C₄-alkoxy, and
R² and R³ have the definitions stated above,

subsequently the compound of the formula (IIId′) is treated with water in the presence of an acid, and the remaining benzylic protective group is removed with hydrogen in the presence of a hydrogenation catalyst or oxidatively.

The treatment of the compounds of the formula (III) mentioned under c.6) with C₁-C₄-alkanol and an alkali metal base and the treatment of the compound of the formula (IIId′) with water in the presence of an acid can be carried out in the manner described for embodiment c.1). If R¹ in the compounds of the formula (III) mentioned under c.6) is a silyl protecting group, the desilylation can also be effected using the desilylating reagents in the manner described above. The benzylic protecting group can be removed in the manner described for embodiment c.3).

According to a further embodiment c.7) of the invention, the compound of the formula (III), in which
R^{a} and R^{b} are the same or different and are benzyl, wherein said benzyl is unsubstituted or optionally has 1, 2 or 3 substituents selected from halogen, C₁-C₄-alkyl or C₁-C₄-alkoxy,
R^{c} is a radical R^{Si},
R¹ is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl, and
R² and R³ have the definitions stated above,

firstly treated with a C₁-C₄-alkanol and an alkali metal base, wherein a compound of the formula (IIIe) is obtained:

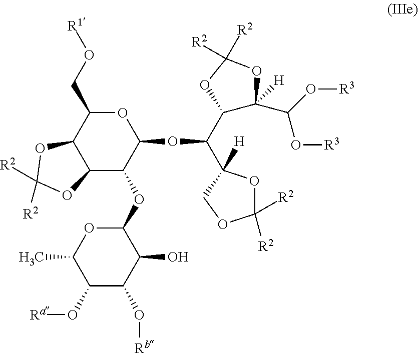

in which
R^{a}″ and R^{b}″ are the same or different and are benzyl, wherein said benzyl is unsubstituted or optionally has 1, 2 or 3 substituents selected from halogen, C₁-C₄-alkyl or C₁-C₄-alkoxy,
R¹′ is benzyl, wherein said benzyl is unsubstituted or optionally has 1, 2 or 3 substituents selected from halogen, hydroxyl, C₁-C₄-alkyl or C₁-C₄-alkoxy, and
R² and R³ have the definitions stated above,

subsequently the compound of the formula (IIIe) is treated with water in the presence of an acid, wherein a compound of the formula (IVb′) is obtained:

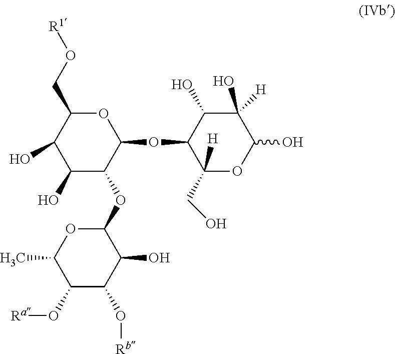

in which
R^{a}″ and R^{b}″ are the same or different and are benzyl, wherein said benzyl is unsubstituted or optionally has 1, 2 or 3 substituents selected from halogen, C₁-C₄-alkyl or C₁-C₄-alkoxy, and
R¹ is benzyl, wherein said benzyl is unsubstituted or optionally has 1, 2 or 3 substituents selected from halogen, hydroxyl, C₁-C₄-alkyl or C₁-C₄-alkoxy,

and subsequently the remaining benzylic protective groups are removed with hydrogen in the presence of a hydrogenation catalyst or oxidatively.

The treatment of the compounds of the formula (III) mentioned under c.7) with C₁-C₄-alkanol and an alkali metal base and the treatment of the compound of the formula (IIIe) with water in the presence of an acid can be carried out in the manner described for embodiment c.1). Alternatively, the desilylation can also be effected using the desilylating reagents in the manner described above. The benzylic protecting groups can be removed in the manner described for embodiment c.3).

According to a further embodiment c.8) of the invention, the compound of the formula (III), in which
R^{a}, R^{b} and R^{c} are the same or different and are benzyl, wherein said benzyl is unsubstituted or optionally has 1, 2 or 3 substituents selected from halogen, C₁-C₄-alkyl or C₁-C₄-alkoxy,
R¹ is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl, and
R² and R³ have the definitions stated above,

is firstly treated with hydrogen in the presence of a hydrogenation catalyst or oxidatively, wherein a compound of the formula (IIIb′) is obtained:

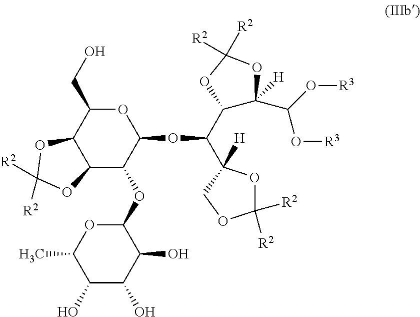

in which
R² and R³ have the definitions stated above, and

subsequently the compound of the formula (IIIb′) is treated with water in the presence of an acid.

The benzylic protecting groups can be removed in the manner described for embodiment c.3). The treatment of the compounds of the formula (IIIb′) with water in the presence of an acid can be effected in the manner described for embodiment c.1).

According to a further embodiment c.9) of the invention, the compound of the formula (III), in which
R^{a} and R^{b} are the same or different and are —C(═O)—C₁-C₆-alkyl, —C(═O)-phenyl, wherein phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, or R^{a} and R^{b} together are a carbonyl radical —(C═O)—,
R^{c} is benzyl, wherein said benzyl is unsubstituted or optionally has 1, 2 or 3 substituents selected from halogen, C₁-C₄-alkyl or C₁-C₄-alkoxy,
R¹ is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl, and
R² and R³ have the definitions stated above,

is firstly treated with a C₁-C₄-alkanol and an alkali metal base, wherein a compound of the formula (IIIf) is obtained:

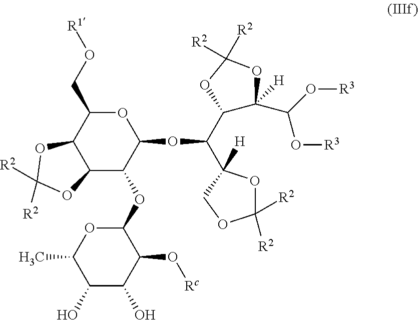

in which
R^{c}′ is benzyl, wherein said benzyl is unsubstituted or optionally has 1, 2 or 3 substituents selected from halogen, C₁-C₄-alkyl or C₁-C₄-alkoxy,
R¹′ is benzyl, wherein said benzyl is unsubstituted or optionally has 1, 2 or 3 substituents selected from halogen, hydroxyl, C₁-C₄-alkyl or C₁-C₄-alkoxy, and
R² and R³ have the definitions stated above,

subsequently the compound of the formula (IIIf) is treated with water in the presence of an acid, wherein a compound of the formula (IVc) is obtained:

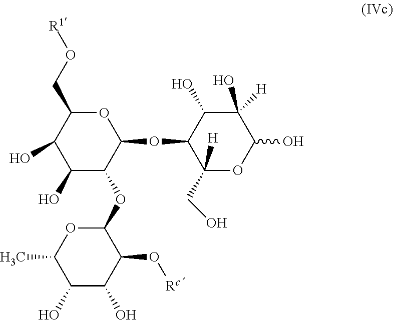

in which
R^{c}′ is benzyl, wherein said benzyl is unsubstituted or optionally has 1, 2 or 3 substituents selected from halogen, C₁-C₄-alkyl or C₁-C₄-alkoxy, and
R¹′ is benzyl, wherein said benzyl is unsubstituted or optionally has 1, 2 or 3 substituents selected from halogen, hydroxyl, C₁-C₄-alkyl or C₁-C₄-alkoxy,

and subsequently the remaining benzylic protective groups are removed with hydrogen in the presence of a hydrogenation catalyst or oxidatively.

The treatment of the compounds of the formula (III) mentioned under c.9) with C₁-C₄-alkanol and an alkali metal base and the treatment of the compound of the formula (IIIf) with water in the presence of an acid can be carried out in the manner described for embodiment c.1). The benzylic protecting groups can be removed in the manner described for embodiment c.3).

According to a further embodiment c.10) of the invention, the compound of the formula (III), in which
R^{a} and R^{b} together are a substituted methylene radical —C(R^{d}R^{e})—, where R^{d} and R^{e} are the same or different and are selected from hydrogen, phenyl and C₁-C₄-alkyl or both radicals R^{d} and R^{e} together are linear C₄-C₆-alkenyl,
R^{c} is benzyl, wherein said benzyl is unsubstituted or optionally has 1, 2 or 3 substituents selected from halogen, C₁-C₄-alkyl or C₁-C₄-alkoxy,
R¹ is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl, and
R² and R³ have the definitions stated above,

is firstly treated with water in the presence of an acid, wherein a compound of the formula (IVc) is obtained:

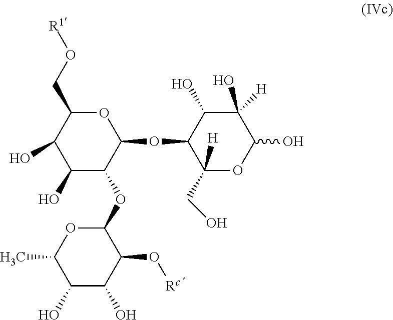

in which
R^{c}′ is benzyl which is unsubstituted or optionally has 1, 2 or 3 substituents selected from halogen, C₁-C₄-alkyl or C₁-C₄-alkoxy, and
R¹′ is benzyl, wherein said benzyl is unsubstituted or optionally has 1, 2 or 3 substituents selected from halogen, hydroxyl, C₁-C₄-alkyl or C₁-C₄-alkoxy,

and subsequently the remaining benzylic protective groups are removed with hydrogen in the presence of a hydrogenation catalyst or oxidatively.

The treatment of the compounds of the formula (III) mentioned under c.10) with water in the presence of an acid can be effected in the manner described for embodiment c.1). The benzylic protecting groups can be removed in the manner described for embodiment c.3).

The 2′-O-fucosyllactose obtained after removal of the protecting groups may be purified by customary purification methods such as chromatography or crystallization, optionally with the aid of additives such as activated carbon, silica gel or polyvinylpyrrolidone. Typical crystallization conditions can be found in Chem. Ber. 1956 11 2513. Depending on the reaction procedure and purification method, the 2″-O-fucosyllactose may still comprise lactose, e.g. in the range from 1% to 20%, based on the product. The chemical purity of the 2′-O-fucosyllactose, excluding lactose, is then generally at least 90%, in particular at least 95% or is even higher. Lactose as impurity is however unproblematic since it is not of concern for use in foodstuffs in these amounts.

In particular, the method according to the invention makes it possible to prepare 2′-O-fucosyllactose such that the content of the undesired β-isomer β-2′-O-fucosyllactose (=β-L-fucopyranosyl)-(1→2)-O-β-D-galactopyranosyl-(1→4)-D-glucopyranose) is already low prior to work-up such that after purification of the reaction product, a 2′-O-fucosyllactose is obtained comprising less than 1% by weight β-2′-O-fucosyllactose, in particular less than 0.5% by weight β-2′-O-fucosyllactose, based on 2′-O-fucosyllactose. This has not been possible to date. Since the method according to the invention, unlike the methods of the prior art, also does not require transition metal catalysts for the hydrogenolytic cleavage of benzyl protecting groups, the transition metals content of the 2′-O-fucosyllactose obtainable according to the invention is often below 1 ppm and especially below the limit of detection.

The compounds of the formula (I) used in step a) of the method according to the invention are novel, if the radicals R^{a}, R^{b} and R^{c} are not all three simultaneously benzyl or 4-methoxybenzyl. Accordingly, the invention further relates to the protected fucose derivatives of the general formula (I′),

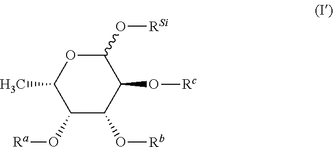

in which
R^{a} and R^{b} are the same or different and are —C(═O)—C₁-C₆-alkyl, —C(═O)-phenyl, wherein phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-alkoxy, or benzyl, wherein said benzyl is unsubstituted or optionally has 1, 2 or 3 substituents selected from halogen, C₁-C₄-alkyl or C₁-C₄-alkoxy, or
R^{a} and R^{b} together are a carbonyl radical —(C═O)— or a substituted methylene radical —C(R^{d}R^{e})—, wherein R^{d} and R^{e} are the same or different and are selected from hydrogen, phenyl and C₁-C₄-alkyl or both radicals R^{d} and R^{e} together are linear C₄-C₆-alkenyl,
R^{c} is a radical R^{Si} or benzyl, wherein benzyl is unsubstituted or optionally has 1, 2 or 3 substituents selected from halogen, C₁-C₄-alkyl or C₁-C₄-alkoxy,
R^{Si} may be the same or different and is a radical of the formula SiR^{f}R^{g}R^{h}, wherein R^{f}, R^{g} and R^{h} are the same or different and are selected from C₁-C₈-alkyl, C₃-C₈-cycloalkyl, phenyl and C₃-C₈-cycloalkyl-C₁-C₄-alkyl,

wherein the radicals R^{a}, R^{b} and R^{c} are not all three simultaneously benzyl or 4-methoxybenzyl.

With regard to preferred and particularly preferred definitions of the radicals R^{a}, R^{b}, R^{c} and R^{Si} in the compounds of the formula (I′), reference is made to that which has been stated above.

Preferred compounds of the general formula (I′) are, for example, selected from

1-O-trimethylsilyl-2,3,4-tri-O-4-Cl-benzylfucopyranose,

1-O-trimethylsilyl-2,3,4-tri-O-2-Cl-benzylfucopyranose,

1-O-trimethylsilyl-2,3,4-tri-O-4-Me-benzylfucopyranose,

1-O-trimethylsilyl-2,3,4-tri-O-(2,4-Cl-benzyl)fucopyranose,

1-O-trimethylsilyl-2-benzyl-3,4-di-O-4-Cl-benzylfucopyranose,

1-O-trimethylsilyl-2-benzyl-3,4-di-O-4-Me-benzylfucopyranose,

1-O-trimethylsilyl-2-benzyl-3,4-di-O-4-OMe-benzylfucopyranose,

1-O-trimethylsilyl-2-benzyl-3,4-di-O-(2,4-Cl-benzyl)fucopyranose,

1-O-trimethylsilyl-2-(4-Cl-benzyl)-3,4-di-O-4-benzylfucopyranose,

1-O-trimethylsilyl-2-(4-Cl-benzyl)-3,4-di-O-4-Cl-benzylfucopyranose,

1-O-trimethylsilyl-2-(4-Cl-benzyl)-3,4-di-O-4-Me-benzylfucopyranose,

1-O-trimethylsilyl-2-(4-Cl-benzyl)-3,4-di-O-4-OMe-benzylfucopyranose,

1-O-trimethylsilyl-2-(4-Cl-benzyl)-3,4-di-O-(2,4-Cl-benzyl)fucopyranose,

1-O-trimethylsilyl-2-(4-Me-benzyl)-3,4-di-O-4-benzylfucopyranose,

1-O-trimethylsilyl-2-(4-Me-benzyl)-3,4-di-O-4-Cl-benzylfucopyranose,

1-O-trimethylsilyl-2-(4-Me-benzyl)-3,4-di-O-4-Me-benzylfucopyranose,

1-O-trimethylsilyl-2-(4-Me-benzyl)-3,4-di-O-4-OMe-benzylfucopyranose,

1-O-trimethylsilyl-2-(4-Me-benzyl)-3,4-di-O-(2,4-Cl-benzyl)fucopyranose,

1-O-trimethylsilyl-2-(4-OMe-benzyl)-3,4-di-O-4-benzylfucopyranose,

1-O-trimethylsilyl-2-(4-OMe-benzyl)-3,4-di-O-4-Cl-benzylfucopyranose,

1-O-trimethylsilyl-2-(4-OMe-benzyl)-3,4-di-O-4-Me-benzylfucopyranose,

1-O-trimethylsilyl-2-(4-OMe-benzyl)-3,4-di-O-4-OMe-benzylfucopyranose,

1-O-trimethylsilyl-2-(4-OMe-benzyl)-3,4-di-O-(2,4-Cl-benzyl)fucopyranose,

1,2-di-O-trimethylsilyl-3,4-di-O-benzylfucopyranose,

1,2-di-O-trimethylsilyl-3,4-di-O-4-Cl-benzylfucopyranose,

1,2-di-O-trimethylsilyl-3,4-di-O-4-F-benzylfucopyranose,

1,2-di-O-trimethylsilyl-3,4-di-O-4-Me-benzylfucopyranose,

1,2-di-O-trimethylsilyl-3,4-di-O-4-OMe-benzylfucopyranose,

1,2-di-O-trimethylsilyl-3,4-di-O-(2,4-Cl-benzyl)fucopyranose,

1-O-trimethylsilyl-2-benzyl-3,4-di-O-benzoylfucopyranose,

1-O-trimethylsilyl-2-benzyl-3,4-di-O-(4-Cl-benzoyl)fucopyranose,

1-O-trimethylsilyl-2-benzyl-3,4-di-O-(4-F-benzoyl)fucopyranose,

1-O-trimethylsilyl-2-benzyl-3,4-di-O-4-Me-benzoylfucopyranose,

1-O-trimethylsilyl-2-benzyl-3,4-di-O-4-OMe-benzoylfucopyranose,

1-O-trimethylsilyl-2-benzyl-3,4-di-O-(2.4-Cl-benzoyl)fucopyranose,

1-O-trimethylsilyl-2-(4-Cl-benzyl)-3,4-di-O-benzoylfucopyranose,

1-O-trimethylsilyl-2-(4-Cl-benzyl)-3,4-di-O-4-Cl-benzoylfucopyranose,

1-O-trimethylsilyl-2-(4-Cl-benzyl)-3,4-di-O-4-F-benzoylfucopyranose,

1-O-trimethylsilyl-2-(4-Cl-benzyl)-3,4-di-O-4-Me-benzoylfucopyranose,

1-O-trimethylsilyl-2-(4-Cl-benzyl)-3,4-di-O-4-OMe-benzoylfucopyranose,

1-O-trimethylsilyl-2-(4-Cl-benzyl)-3,4-di-O-(2,4-Cl-benzoyl)fucopyranose,

1-O-trimethylsilyl-2-(4-Me-benzyl)-3,4-di-O-benzoylfucopyranose,

1-O-trimethylsilyl-2-(4-Me-benzyl)-3,4-di-O-4-Cl-benzoylfucopyranose,

1-O-trimethylsilyl-2-(4-Me-benzyl)-3,4-di-O-4-F-benzoylfucopyranose,

1-O-trimethylsilyl-2-(4-Me-benzyl)-3,4-di-O-4-Me-benzoylfucopyranose,

1-O-trimethylsilyl-2-(4-Me-benzyl)-3,4-di-O-4-OMe-benzoylfucopyranose,

1-O-trimethylsilyl-2-(4-Me-benzyl)-3,4-di-O-(2,4-Cl-benzoyl)fucopyranose,

1-O-trimethylsilyl-2-(4-OMe-benzyl)-3,4-di-O-benzoylfucopyranose,

1-O-trimethylsilyl-2-(4-OMe-benzyl)-3,4-di-O-4-Cl-benzoylfucopyranose,

1-O-trimethylsilyl-2-(4-OMe-benzyl)-3,4-di-O-4-F-benzoylfucopyranose,

1-O-trimethylsilyl-2-(4-OMe-benzyl)-3,4-di-O-4-Me-benzoylfucopyranose,

1-O-trimethylsilyl-2-(4-OMe-benzyl)-3,4-di-O-4-OMe-benzoylfucopyranose,

1-O-trimethylsilyl-2-(4-OMe-benzyl)-3,4-di-O-(4-Cl-benzoyl)fucopyranose,

1,2-di-O-trimethylsilyl-3,4-di-O-benzoylfucopyranose,

1,2-di-O-trimethylsilyl-3,4-di-O-(4-Cl-benzoyl)fucopyranose,

1,2-di-O-trimethylsilyl-3,4-di-O-(4-F-benzoyl)fucopyranose,

1,2-di-O-trimethylsilyl-3,4-di-O-4-Me-benzoylfucopyranose,

1,2-di-O-trimethylsilyl-3,4-di-O-4-OMe-benzoylfucopyranose,

1,2-di-O-trimethylsilyl-3,4-di-O-(2,4-Cl-benzoyl)fucopyranose.

The compounds of the formula (I) used in step a) are preferably prepared by reacting a fucose of the general formula (I-1),

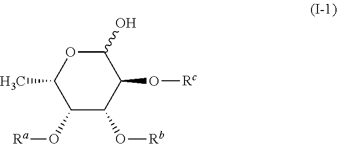

where R^{a}, R^{b} and R^{c} have the definitions stated above, with a silyl chloride of the general formula Cl-SiR^{f}R^{g}R^{h}, where R^{f}, R^{g} and R^{h} are the same or different and are selected from C₁-C₈-alkyl, C₃-C₈-cycloalkyl, phenyl and C₁-C₈-cycloalkyl-C₁-C₄-alkyl.

By way of preference, radicals R^{f}, R^{g} and R^{h} in the silyl chloride compounds of the general formula C₁-SiR^{f}R^{g}R^{h} are the same or different and in particular are C₁-C₄-alkyl, especially methyl.

The reaction of the compounds of the formula (I-1) to give the compounds (I) typically takes place in the presence of a base and is aligned with the procedure for the persilylation of fucose described in, for example, Synlett, 1996(6), pp. 499-501.

For this purpose, suitable or preferred bases are, for example, the bases specified in the context of the reaction in step b) of the method according to the invention.

In order to avoid secondary reactions, the base is preferably used in at least an equimolar amount, based on the compound of the formula (I-1). The base is typically used in an amount of 1 to 3 mol per mole of the compound of the formula (I-1), preferably in an amount of 1 to 1.5 mol per mole of the compound of the formula (I-1), especially in an amount of 1 to 1.2 mol per mole of the compound of the formula (I-1).

The molar ratio between the compound of the formula (I-1) and the silyl chloride compound of the general formula Cl-SiR^{f}R^{g}R^{h} in the reaction is typically 1:1.5, particularly preferably 1:1.2, especially 1:1.1.

The compound of the formula (I-1) is generally reacted with the silyl chloride compound of the general formula Cl-SiR^{f}R^{g}R^{h} in an inert organic solvent or diluent. Preference is given to aprotic solvents, particularly those having a low content of protic impurities such as water, alcohols or acid. Preferred solvents are, for example, the solvents specified above in the context of the reaction in step a) of the method according to the invention. Particularly preferred solvents are haloalkanes such as dichloromethane, trichloromethane, dichloroethane or aromatic hydrocarbons such as toluene and xylenes. Especially preferred are dichloromethane and toluene.

The compounds of the formula (I-1) having free OH groups at the anomeric center required for preparing the compounds of the formula (I) used in step a) are prepared either by the methods known from the prior art or may be prepared by methods analogous to the methods known therein. For example, compounds of the formula (I-1), in which the radicals R^{a}, R^{b} and R^{c} are identical and are benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl, are prepared analogously to the methods described in Carbohydrate Research (1993), 245(2), pp. 193-218, WO 2010070616, WO 2014130613 and the literature cited therein. Compounds of the formula (I-1), in which either only the radical R^{c} is benzyl or the radicals R^{a}, R^{b} and R^{c} bear different benzyl groups, may be prepared, for example, by the methods described in WO 2010070616, WO 2012113404 or in Mendeleev Communications (1999), (3), pp. 114-116. Compounds of the formula (I-1), in which R^{a} and R^{b} are different, may also be prepared, for example, analogously to the methods described in J. Org. Chem., 1984, 49 (6), pp. 992-996.

As an alternative to the preparation methods described above, the compounds of the formula (I), wherein the radical R^{c} is R^{Si} and both radicals R^{Si} are the same, can also be prepared by reacting a fucose of the general formula (I-2),

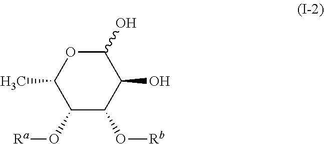

where R^{a} and R^{b} have the definitions stated above, with a silyl chloride of the general formula Cl-SiR^{f}R^{g}R^{h}, where R^{f}, R^{g} and R^{h} are the same or different and are selected from C₁-C₈-alkyl, C₃-C₈-cycloalkyl, phenyl and C₃-C₈-cycloalkyl-C₁-C₄-alkyl.

The reaction of the compounds of the formula (I-2) to give compounds (I) is carried out for the most part analogously to the reaction of the compounds of the formula (I-1).

However, the base used in the reaction of the compounds of the formula (I-2) is typically used in an amount of 1 to 5 mol per mole of the compound of the formula (I-2), preferably in an amount of 1 to 3 mol per mole of the compound of the formula (I-2), especially in an amount of 1 to 2.4 mol per mole of the compound of the formula (I-2).

The molar ratio between the compound of the formula (I-1) and the silyl chloride compound of the general formula Cl-SiR^{f}R^{g}R^{h} in the reaction of the compounds of the formula (I-2) is typically 1:3, more preferably 1:2.4, in particular 1:2.2.

The compounds of the formula (I.a) obtained in step a) of the method according to the invention are likewise novel, if the radicals R^{a}, R^{b} and R^{c} are not all three simultaneously benzyl and, if R^{a} and R^{b} together form a dimethylmethylene radical —C(CH₃CH₃)—, R^{c} is not a tert-butyldimethylsilyl radical.

Accordingly, the invention further relates to the protected 1-iodofucose derivatives of the general formula (I.a′),

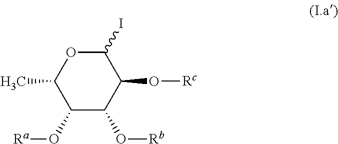

in which
R^{a} and R^{b} are the same or different and are —C(═O)-C₁-C₆-alkyl, —C(═O)-phenyl, wherein phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, or benzyl, wherein said benzyl is unsubstituted or optionally has 1, 2 or 3 substituents selected from halogen, C₁-C₄-alkyl or C₁-C₄-alkoxy, or
R^{a} and R^{b} together are a carbonyl radical —(C═O)— or a substituted methylene radical —C(R^{d}R^{e})—, wherein R^{d} and R^{e} are the same or different and are selected from hydrogen, phenyl and C₁-C₄-alkyl or both radicals R^{d} and R^{e} together are linear C₄-C₆-alkenyl,
R^{c} is an radical R^{Si} or benzyl, wherein benzyl is unsubstituted or optionally has 1, 2 or 3 substituents selected from halogen, C₁-C₄-alkyl or C₁-C₄-alkoxy,
R^{Si} may be the same or different and is a radical of the formula SiR^{f}R^{g}R^{h}, wherein R^{f}, R^{g} and R^{h} are the same or different and are selected from C₁-C₈-alkyl, C₃-C₈-cycloalkyl, phenyl and C₃-C₈-cycloalkyl-C₁-C₄-alkyl,

wherein the radicals R^{a}, R^{b} and R^{h} are not all three simultaneously benzyl and, in the case that R^{a} and R^{b} together form a dimethylmethylene radical —C(CH₃CH₃)—, R^{c} is not a tert-butyldimethylsilyl radical.

With regard to preferred and particularly preferred definitions of the radicals R^{a}, R^{b}, R^{c}and R^{Si} in the compounds of the formula (I.a′), reference is made to that which has been stated above.

Preferred compounds of the general formula (I.a′) are, for example, selected from 1-deoxy-2,3,4-tri-O-4-Cl-benzylfucopyranosyl iodide,

1-deoxy-2,3,4-tri-O-2-Cl-benzylfucopyranosyl iodide,

1-deoxy-2,3,4-tri-O-4-Me-benzylfucopyranosyl iodide,

1-deoxy-2,3,4-tri-O-4-OMe-benzylfucopyranosyl iodide,

1-deoxy-2,3,4-tri-O-(2,4-Cl-benzyl)fucopyranosyl iodide,

1-deoxy-2-benzyl-3,4-di-O-4-Cl-benzylfucopyranosyl iodide,

1-deoxy-2-benzyl-3,4-di-O-4-Me-benzylfucopyranosyl iodide,

1-deoxy-2-benzyl-3,4-di-O-4-OMe-benzylfucopyranosyl iodide,

1-deoxy-2-benzyl-3,4-di-O-(2,4-Cl-benzyl)fucopyranosyl iodide,

1-deoxy-2-(4-Cl-benzyl)-3,4-di-O-4-benzylfucopyranosyl iodide,

1-deoxy-2-(4-Cl-benzyl)-3,4-di-O-4-Cl-benzylfucopyranosyl iodide,

1-deoxy-2-(4-Cl-benzyl)-3,4-di-O-4-Me-benzylfucopyranosyl iodide,

1-deoxy-2-(4-Cl-benzyl)-3,4-di-O-4-OMe-benzylfucopyranosyl iodide,

1-deoxy-2-(4-Cl-benzyl)-3,4-di-O-(2,4-Cl-benzyl)fucopyranosyl iodide,

1-deoxy-2-(4-Me-benzyl)-3,4-di-O-4-benzylfucopyranosyl iodide,

1-deoxy-2-(4-Me-benzyl)-3,4-di-O-4-Cl-benzylfucopyranosyl iodide,

1-deoxy-2-(4-Me-benzyl)-3,4-di-O-4-Me-benzylfucopyranosyl iodide,

1-deoxy-2-(4-Me-benzyl)-3,4-di-O-4-OMe-benzylfucopyranosyl iodide,

1-deoxy-2-(4-Me-benzyl)-3,4-di-O-(2,4-Cl-benzyl)fucopyranosyl iodide,

1-deoxy-2-(4-OMe-benzyl)-3,4-di-O-4-benzylfucopyranosyl iodide,

1-deoxy-2-(4-OMe-benzyl)-3,4-di-O-4-Cl-benzylfucopyranosyl iodide,

1-deoxy-2-(4-OMe-benzyl)-3,4-di-O-4-Me-benzylfucopyranosyl iodide,

1-deoxy-2-(4-OMe-benzyl)-3,4-di-O-4-OMe-benzylfucopyranosyl iodide,

1-deoxy-2-(4-OMe-benzyl)-3,4-di-O-(2,4-Cl-benzyl)fucopyranosyl iodide,

1-deoxy-2-O-trimethylsilyl-3,4-di-O-benzylfucopyranosyl iodide,

1-deoxy-2-O-trimethylsilyl-3,4-di-O-4-Cl-benzylfucopyranosyl iodide,

1-deoxy-2-O-trimethylsilyl-3,4-di-O-4-Me-benzylfucopyranosyl iodide,

1-deoxy-2-O-trimethylsilyl-3,4-di-O-4-OMe-benzylfucopyranosyl iodide,

1-deoxy-2-O-trimethylsilyl-3,4-di-O-(2,4-Cl-benzyl)fucopyranosyl iodide,

1-deoxy-2-benzyl-3,4-di-O-benzoylfucopyranosyl iodide,

1-deoxy-2-benzyl-3,4-di-O-(4-Cl-benzoyl)fucopyranosyl iodide,

1-deoxy-2-benzyl-3,4-di-O-(4-F-benzoyl)fucopyranosyl iodide,

1-deoxy-2-benzyl-3,4-di-O-4-Me-benzoylfucopyranosyl iodide,

1-deoxy-2-benzyl-3,4-di-O-4-OMe-benzoylfucopyranosyl iodide,

1-deoxy-2-benzyl-3,4-di-O-(2,4-Cl-benzoyl)fucopyranosyl iodide,

1-deoxy-2-(4-Cl-benzyl)-3,4-di-O-benzoylfucopyranosyl iodide,

1-deoxy-2-(4-Cl-benzyl)-3,4-di-O-4-Cl-benzoylfucopyranosyl iodide,

1-deoxy-2-(4-Cl-benzyl)-3,4-di-O-4-F-benzoylfucopyranosyl iodide,

1-deoxy-2-(4-Cl-benzyl)-3,4-di-O-4-Me-benzoylfucopyranosyl iodide,

1-deoxy-2-(4-Cl-benzyl)-3,4-di-O-4-OMe-benzoylfucopyranosyl iodide,

1-deoxy-2-(4-Cl-benzyl)-3,4-di-O-(2,4-Cl-benzoyl)fucopyranosyl iodide,

1-deoxy-2-(4-Me-benzyl)-3,4-di-O-benzoylfucopyranosyl iodide,

1-deoxy-2-(4-Me-benzyl)-3,4-di-O-4-Cl-benzoylfucopyranosyl iodide,

1-deoxy-2-(4-Me-benzyl)-3,4-di-O-4-F-benzoylfucopyranosyl iodide,

1-deoxy-2-(4-Me-benzyl)-3,4-di-O-4-Me-benzoylfucopyranosyl iodide,

1-deoxy-2-(4-Me-benzyl)-3,4-di-O-4-OMe-benzoylfucopyranosyl iodide,

1-deoxy-2-(4-Me-benzyl)-3,4-di-O-(2,4-Cl-benzoyl)fucopyranosyl iodide,

1-deoxy-2-(4-OMe-benzyl)-3,4-di-O-benzoylfucopyranosyl iodide,

1-deoxy-2-(4-OMe-benzyl)-3,4-di-O-4-Cl-benzoylfucopyranosyl iodide,

1-deoxy-2-(4-OMe-benzyl)-3,4-di-O-4-F-benzoylfucopyranosyl iodide,

1-deoxy-2-(4-OMe-benzyl)-3,4-di-O-4-Me-benzoylfucopyranosyl iodide,

1-deoxy-2-(4-OMe-benzyl)-3,4-di-O-4-OMe-benzoylfucopyranosyl iodide,

1-deoxy-2-(4-OMe-benzyl)-3,4-di-O-(2,4-Cl-benzoyl)fucopyranosyl iodide,

1-deoxy-2-O-trimethylsilyl-3,4-di-O-benzoylfucopyranosyl iodide,

1-deoxy-2-O-trimethylsilyl-3,4-di-O-(4-Cl-benzoyl)fucopyranosyl iodide,

1-deoxy-2-O-trimethylsilyl-3,4-di-O-(4-F-benzoyl)fucopyranosyl iodide,

1-deoxy-2-O-trimethylsilyl-3,4-di-O-4-Me-benzoylfucopyranosyl iodide,

1-deoxy-2-O-trimethylsilyl-3,4-di-O-4-OMe-benzoylfucopyranosyl iodide,

1-deoxy-2-O-trimethylsilyl-3,4-di-O-(2,4-Cl-benzoyl)fucopyranosyl iodide,

Compounds of the formula (II), where R¹ is a radical C(═O)—R¹¹, are known, e.g. from the references cited at the outset, or from Tetrahedron Letters, 1981, 22 (50), 5007-5010, WO 2010/115934, WO 2010/115935 and Carbohydrate Research, 1981, 88, 51-60, or may be prepared in analogy to the methods described therein.

Compounds of the formula (II), where R¹ is a radical SiR¹²R¹³R¹⁴, may be prepared in a simple manner by selective silylation of the CH₂—OH group of the compounds of the formula (II-1).

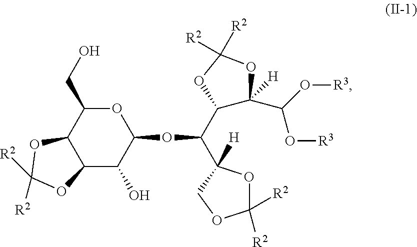

R² and R³ in formula (II-1) are as defined above, particularly as defined below:
R² is in particular C₁-C₄-alkyl and especially methyl, or two radicals R² attached to the same carbon atom are together 1,5-pentanediyl and thus form a radical cyclohexane-1,1-diyl with the carbon atom to which they are attached. All radicals R² are especially methyl.
R³ is particularly C₁-C₄-alkyl and especially methyl.

For the selective silylation, the compound of the formula (II-1) is typically reacted with a suitable silylating reagent, e.g. a compound of the formula SiXR¹²R¹³R¹⁴, where R¹², R¹³ and R¹⁴ are as defined previously and are especially methyl and X is halogen, particularly chlorine. The reaction with the silylating reagent is preferably carried out in the presence of a base.

For the selective silylation, 0.9 to 2 mol, particularly 1 to 1.5 mol, especially about 1.1 mol of the silylating reagent is typically used per mole of the compound of the formula (II-1).

In order for the reaction to proceed selectively, the reaction of (II-1) is preferably carried out in the temperature range from −40 to +40° C., particularly in the range from −20 to +20° C., especially preferably in the range from −5 to +5° C., e.g. at about 0° C.

Suitable bases are primarily amine bases, particularly secondary and tertiary amines, especially pyridine bases and tertiary aliphatic or cycloaliphatic amines. Suitable pyridine bases are, for example, pyridine, quinoline and C₁-C₆-alkyl-substituted pyridines, particularly mono-, di- and tri(C₁-C₆-alkyl)pyridines such as 2,6-di(C₁-C₆-alkyl)pyridines and collidines. Suitable tertiary aliphatic or cycloaliphatic amines are tri(C₁-C₆-alkyl)amines such as triethylamine, diisopropylmethylamine, tri-n-butylamine or isopropyldimethylamine, C₃-C₈-cycloalkyl-di(C₁-C₆-alkyl)amines such as cyclohexyldimethylamine, N—(C₁-C₆-alkyl)piperidine such as N-methylpiperidine and di(C₃-C₈-cycloalkyl)-C₁-C₆-alkylamines such as biscyclohexylmethylamine.

The base is typically used in an amount of 0.9 to 2 mol, particularly in an amount of 1 to 1.5 mol per mole of the compound of the formula (II-1).

The compound of the formula (II-1) is reacted with the silylating reagent, generally in an inert organic solvent or diluent. Preference is given to aprotic solvents, particularly those having a low content of erotic impurities such as water, alcohols or acid. Preferred organic solvents are haloalkanes, such as dichloromethane, trichloromethane, dichloroethane, aromatic hydrocarbons such as toluene and xylenes, dialkyl ethers such as diethyl ether, diisopropyl ether, methyl tert-butyl ether, cyclic ethers such as tetrahydrofuran or dioxane, dialkylamides of aliphatic carboxylic acids such as dimethylformamide or dimethylacetamide and also alkyl nitriles such as acetonitrile, and also mixtures of the abovementioned solvents. The solvent is preferably selected such that all constituents are present in dissolved form.

The compounds of the formula (II-1) are known, e.g. from Carbohydrate Research, 212 (1991), pp. C1-C3; Tetrahedron Lett., 31 (1990) 4325; Carbohydrate Research, 75 (1979) C11; Carbohydrate Research, 88 (1981) 51; Chem. 5 (1999) 1512; WO 2010/070616, WO 2012/113404, WO 2010/115934 and WO 2010/115935 or may be prepared by the methods described therein.

Compounds of the formula (II), where R¹ is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl, can also be prepared in a simple manner by selective benzylation of the CH₂—OH group of the compounds of the formula (II-1). Appropriate methods are known to those skilled in the art, for example from P. G. M. Wuts et al., “Greene's Protecting Groups in Organic Synthesis”, 4th Edition, Wiley 2006 and the literature cited therein, or the literature cited at the outset for preparing 2′-O-fucosylactose.

Compounds of the formula (III), in which R^{a} and RD is benzyl or acyl and R^{c} is benzyl, which is unsubstituted or optionally has 1, 2 or 3 substituents, are known from the literature, e.g. from Carbohydrate Research, 1991, Vol. 212, pp. 1-11, Carbohydrate Research, 1981, Vol. 88, pp. 51-60, IT 1392456, WO 2010 070616, WO 2013004669, J. Carb. Chem., 2001, Vol. 20, pp. 611-636, WO 2010115934, WO 2010115935. Some of the compounds of the formula (III) and also partially protected compounds thereof are novel however.

Accordingly, the invention further relates to the protected 2′-O-fucosyllactose derivatives of the general formula (IIIa),

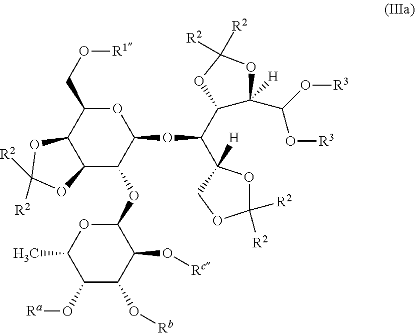

in which
R^{a}, R^{b}, R² and R³ are as defined above;
R^{c}″ is hydrogen or a radical R^{Si},
R¹″ is hydrogen, a radical —C(═O)—R¹¹ or a radical SiR¹²R¹³R¹⁴, in which
   R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
   R¹², R¹³ and R¹⁴ are the same or different and are selected from C₁-C₈-alkyl, C₃-C₈-cycloalkyl, phenyl and C₃-C₈-cycloalkyl-C₁-C₄-alkyl,
   or

is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl, and
R² and R³ have the definitions stated above.

The invention further relates to the protected 2¹-O-fucosyllactose derivatives of the general formula (IIIb),

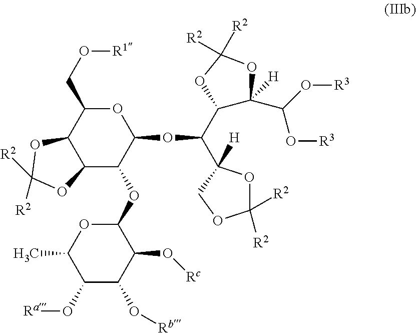

in which
R^{a}′″ and R^{b}′″ together are a carbonyl radical —(C═O)— or a substituted methylene radical —C(R^{d}R^{e})—, wherein R^{d} and R^{e} are the same or different and are selected from hydrogen, phenyl and C₁-C₄alkyl or both radicals R^{d} and R^{e} together are linear C₄-C₆-alkenyl,
R^{c} is as defined above,
R¹″ is hydrogen, a radical —C(═O)—R¹¹ or a radical SiR¹²R¹³R¹⁴, where
   R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄haloalkyl and C₁-C₄-haloalkoxy, and
   R¹², R¹³ and R¹⁴ are the same or different and are selected from C₁-C₈-alkyl, C₃-C₈-cycloalkyl, phenyl and C₃-C₈-cycloalkyl-C₁-C₄alkyl,
   or

is benzyl, wherein said benzyl is unsubstituted or optionally has 1, 2 or 3 substituents selected from halogen, C₁-C₄-alkyl, C₁-C₄alkoxy or —O—C(═O)—C₁-C₄-alkyl, and
R² and R³ have the definitions stated above.

The invention further relates to the partially protected 2′-O-fucosyllactose derivatives of the general formula (IVa),

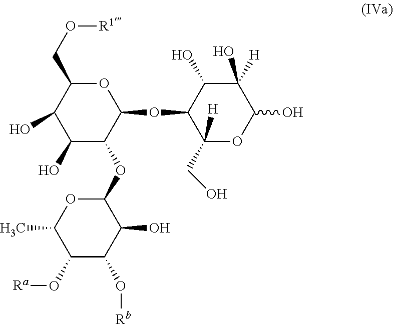

in which
R^{a} and R^{b} have the definitions stated above,
R¹′″ is hydrogen, a radical —C(═O)—R¹¹, where
   R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄alkyl, C₁-C₄-alkoxy, C₁-C₄haloalkyl and C₁-C₄-haloalkoxy,
   or

is benzyl, wherein said benzyl is unsubstituted or optionally has 1, 2 or 3 substituents selected from halogen, C₁-C₄alkyl, C₁-C₄-alkoxy or —O—C(═O)—C₁-C₄-alkyl.

The invention further relates to the partially protected 2′-O-fucosyllactose derivatives of the general formula (IVb),

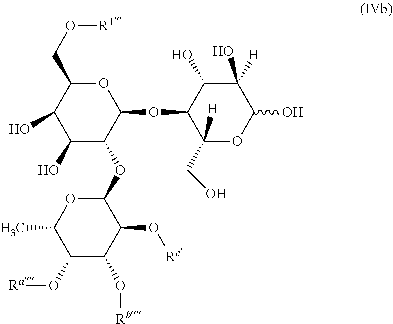

in which
R^{a}″″ and R^{b}″″ together are a carbonyl radical —(C═O)—,
R^{c}′ is benzyl which is unsubstituted or optionally has 1, 2 or 3 substituents selected from halogen, C₁-C₄-alkyl or C₁-C₄-alkoxy,
R¹′″ is hydrogen, a radical —C(═O)—R¹¹, where
   R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy,
   or

is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl.

With regard to preferred and particularly preferred definitions of the radicals R^{a}, R^{b}, R^{c}, R^{a}′″, R^{b}′″, R^{a}″″, R^{b}″″, R^{c}″, R¹″, R² and R³ in the compounds of the formulae (IIIa), (IIIb), (IVa) and (IVb), reference is made to that which has been stated above.

As already mentioned, the advantage of the method according to the invention is that, in particular, the undesired β-isomer is not formed, or is formed to a very much lower extent, than in the methods of the prior art. The method and the reactants of the formulae (I′) and (I.a′) obtained by the method and also the intermediates of the formulae (IIIa), (IIIb), (IVa) and (IVb) are, therefore, particularly suitable for preparing 2′-O-fucosyllactose. Accordingly, the present invention relates also to the use of compounds of the general formulae (I′), (I.a′), (IIIa), (IIIb), (IVa) or (IVb), as defined above, for preparing 2′-O-fucosyllactose.

As already mentioned, the 2′-O-fucosyllactose obtainable by the method according to the invention, in comparison to the known 2′-O-fucosyllactose, is characterized in that it does not comprise, or only comprises in much lower fractions, those impurities which cannot be removed.

Accordingly, the present invention relates to the use of at least one of the compounds of the general formulae (I′), (I.a′), (IIIa), (IIIb), (IVa) or (IVb), as defined above, for preparing foodstuffs and food additives, comprising the preparation of 2′-O-fucosyllactose from at least one of the compounds of the general formulae (I′), (I.a′), (IIIa), (IIIb), (IVa) or (IVb).

Examples of foodstuffs in which the 2′-O-fucosyllactose, prepared by using at least one of the compounds of the general formulae (I′), (I.a′), (IIIa), (IIIb), (IVa) or (IVb), can be used are familiar to those skilled in the art, e.g. from the prior art cited at the outset. Here, this can take the form of compositions based on naturally occurring products, e.g. dairy products, and also artificially prepared formulations, for example, for dietary or medicinal nutrition. The latter can be ready-to-use formulations and can be used directly, or may take the form of concentrated formulations, e.g. liquid or semi-solid concentrates, or solid products such as granules, flakes or powder which are converted into a ready-to-use formulation before use by addition of liquid, particularly water, or which are incorporated into a conventional foodstuff.

The concentrates and also the ready-to-use formulations can be solid, liquid or semi-solid formulations.

In particular, the foodstuffs in which the 2′-O-fucosyllactose prepared by using at least one of the compounds of the general formulae (I′), (I.a′), (IIIa), (IIIb), (IVa) or (IVb) is used, are foodstuff compositions for child nutrition, particularly in baby formula and especially infant formula.

In general, the foodstuffs in which the 2¹-O-fucosyllactose prepared by using at least one of the compounds of the general formulae (I′), (I.a′), (IIIa), (IIIb), (IVa) or (IVb) is used, are solid, semi-solid or liquid foodstuff compositions, particularly semi-solid or especially liquid foodstuff compositions.

The foodstuff compositions, i.e. the ready-to-use foodstuff compositions and the concentrates, may be prepared in a manner known per se by incorporating the 2′-O-fucosyllactose, which has been prepared from at least one of the compounds of the general formulae (I′), (I.a′), (IIIa), (IIIb), (IVa) or (IVb), into a foodstuff formulation. This foodstuff formulation may comprise other nutrients, in addition to the 2′-O-fucosyllactose, and generally comprises at least one carrier suitable for foodstuff, wherein the latter may be solid, liquid or semi-solid. The carrier can be a foodstuff or a substance with nutritional value, or it may be a substance which itself has no nutritional value, e.g. dietary fiber or water.

The examples which follow serve to illustrate the invention.

The following abbreviations were used:
d: doublet
s: singlet
t: triplet
m: multiplet
DCM: Dichloromethane—preferably stabilized with amylene or without stabilizer
DMF: Dimethylformamide
of th: of theory
eq: Molar equivalents
MeOH: Methanol
NEt₃: Triethylamine
RT: Ambient temperature, about 22° C.
Rt: Retention time
TMS: Trimethylsilyl
TMSI: Trimethylsilyl iodide
2′-FL: 2′-O-fucosyllactose

Unless stated otherwise, HPLC analysis was carried out using an Agilent Series 1200 and a Gemini-NX column (3 μm; 250×4.6 mm). The column was maintained at 35° C. and operated at 160 bar.

Acetonitrile/water 65/35 v/v was used as eluent; detection was with an RID detector. The flow rate was 1 ml/min, the run time 10 to 40 min. The sample volume was 5 μl. For the sample preparation, 10 mg of sample were in each case dissolved in 1 ml of acetonitrile/water in a 65/35 ratio by volume.

### EXAMPLES

### Example 1

### (Preparation Example): Preparation of 3,4-O-isopropylidenefucose (Method A)

9.95 g of L-fucose (60 mmol) were stirred in 118.7 g (2.04 mol) of acetone with 19.5 g of CuSO₄ for 16 h. The reaction mixture was then filtered and the solid obtained was washed twice each time with 50 ml of acetone. The solid was dried for 5 h under vacuum, resuspended in 150 ml of acetone and then stirred at RT for 16 h.

The suspension was then filtered again and the solid obtained was washed twice each time with 50 ml of acetone. The filtrates were combined and the volatile constituents were removed under reduced pressure. 3.0 g of the residue were chromatographed on silica gel 60 (particle size 0.04-0.063 mm, column bed volume 580 ml) with ethyl acetate. This gave 1.2 g of the title compound.

NMR as a mixture of the anomeric α and β forms:

¹³C-NMR(CD2Cl₂, 500 MHz): δ (ppm) 110.07, 109.46, 96.58, 91.86, 79.25, 76.79, 76.14, 74.96, 75.89, 69.77, 69.46, 64.33, 28.35, 27.80, 26.40, 25.93, 16,78m, 16.65.

### Example 2

### (Preparation Example): Preparation of 3,4-O-isopropylidenefucose (Method B)

9.95 g (60 mmol) of L-fucose were charged in 100 ml of DMF at RT. The mixture was cooled to 0° C. and 4.55 g (1 eq.) of 2-methoxypropene were slowly added dropwise; 65 mg (0.004 eq., based on the fucose used) of camphorsulfonic acid were added and the mixture was stirred at 0° C. for 1 h. Subsequently, a further 4.55 g (1 eq.) of 2-methoxypropene were slowly added dropwise and the mixture was stirred at 0° C. for 2 h. 10 g of sodium carbonate were then slowly added and the mixture stirred at RT for 1 h.

The resulting solid was filtered off and the filtrate was concentrated under reduced pressure. 18.9 g of the residue were chromatographed on silica gel 60 (particle size 0,04-0.063 mm, column bed volume 1300 ml) with ethyl acetate. 2.3 g of the title compound were obtained which is identical according to HPLC with that from example 1.

### Example 3

### Preparation of 4-O-(3,4-isopropylidene-β-D-galactopyranosyl)-2,3; 5,6-bis-O-isopropylidene-D-glucose dimethyl acetal (Compound II-3: Compound of the Formula (II) Where R¹═H, R²═CH₃ and R³═CH₃)

205.4 g (0.6 mol) of lactose were charged in 409 ml of 1,4-dioxane. To this were added 28.44 g (0.12 mol =0.2 eq.) of DL-camphorsulfonic acid and 376.4 ml (3 mol=5 eq.) of dimethoxypropane. The mixture was heated under reflux for 4 h. 10.04 ml of triethylamine were then added. After cooling, the mixture was concentrated under reduced pressure (2 mbar) and 50° C., during which two times 300 ml of toluene each time were added and codistilled. The residue that remained was taken up in 1000 ml of methanol/water 9: 1 v/v and stirred at 60° C. for 1 h. After removing the methanol under reduced pressure, 600 ml of DCM were added and the resulting solution was washed twice with 5% aqueous NaHCO₃ solution. After removal of the solvent under reduced pressure, the residue was taken up in 50 ml of ethyl acetate and was crystallized at −10° C. with addition of 50 ml of cyclohexane and 160 ml of diisopropyl ether. Filtration and washing of the crystals twice with 50 ml of cold diisopropyl ether affords 118.9 g of the title compound with a purity of 92%.

¹H-NMR (CD₂Cl₂): δ 4.5 (t, 1H), 4.4 (d, 1H), 4.4-4.3 (m, 2H), 4.2 (m, 1H), 4.1-3.8 (m, 7H,), 3.6 (m, 1H), 3.5 (m, 1H), 3.4 (s, 6H), 3.3 (d, 1H), 2.9 (s, 1H), 1.5 (2 s, 6H), 1.4 (s, 6H), 1.3 (s, 6H).

### Example 4

### Preparation of 4-O-(6-O-acetyl-3,4-isopropylidene-β-D-galactopyranosyl)-2,3; 5,6-bis-O-isopropylidene-D-glucose dimethyl acetal (Compound II-4: Compound of the Formula (II) Where R¹=acetyl, R²═CH₃ and R³═CH₃)

58.8 g (92% strength=0.106 mol) of compound II-3 from example 3 were dissolved in 183 ml of DCM. The solution was treated with 25.12 ml (0.181 mol) of NEt₃ and cooled to −5° C. To this was added dropwise a solution of 60.9 g (0.16 mol) of acetyl chloride dissolved in 61 ml of DCM over a period of 70 min. and the resulting mixture was stirred at 0° C. for 20 h. For the work-up, the mixture was treated with 100 ml of icewater, the phases were separated and the aqueous phase was extracted twice with 50 ml of DCM each time.

The combined organic phases were washed successively with 50 ml of 1N aqueous hydrochloric acid, 50 ml of 5% aqueous NaHCO₃ solution, dried over Na₂SO₄ and concentrated under reduced pressure (250 mbar) at 40° C.

The title compound II-4 was obtained in an amount of 65.1 g with a purity of 73%. The product was reacted further directly or was purified to 90% purity by chromatography or crystallization of the secondary components from cyclohexane.

¹H-NMR (CD₂Cl₂): δ 4.5-4.4 (m, 2H), 4.4 (m, 1H), 4.4-4.2 (m, 2H), 4.2-4.1 (m, 2H), 4.1-3.9 (m, 5H), 3.5 (m, 1H), 3.4 (2 s, 6H), 2.1 (s 3H), 1.5 (s 2 6H), 1.4 (2 s 6H) 1.3 s, 6H).

### Example 5

### (Preparation Example): Preparation of 1,2-di-O-trimethylsilyl-3,4-O-isopropylidenefucose

3.4 g (15 mmol) of 90% strength acetonide (3,4-O-isopropylidenefucose) from examples 1 and 2 were dissolved in 15.7 g of DMF to which 3.34 g (33 mmol) of NEt₃ were added and the mixture was cooled to 0° C. 3.54 g (32 mmol) of chlorotrimethylsilane were slowly added dropwise over 15 min. at −5 to 0° C. and the mixture was stirred at 0° C. for 4 h. 25 ml of pentane were added to the reaction mixture and stirred at −5 to 0° C. for 10 min. 17 ml of cold H₂O were then slowly added dropwise at −5 to 0° C. and the resulting phases separated. The organic phase was washed 3 times each with 10 ml of H₂O and once with 10 ml of saturated NaCl solution, dried over Na₂SO₄ and concentrated under reduced pressure. This gave 4.1 g of the title compound as crude product with a purity by NMR of 90%.

¹³C-NMR(CD₂Cl₂, 500 MHz): δ (ppm) 108.76, 94.02, 77.12, 76.55, 72.64, 63.26, 28.54, 26.49, 16.58, 0.44, 0.44, 0.44, 0.08, 0.08, 0.08.

### Example 6

### Preparation of the Compound of the Formula (III) Where R¹=acetyl, R²═CH₃, R³═CH₃, R^{a} and R^{b}═—C(CH₃)₂— and R^{c}═—Si(CH₃)₃

2.06 g (10 mmol) of TMSI were added to 3.87 g (10 mmol) of disilyl compound (1,2-di-O-trimethylsilyl-3,4-O-isopropylidenefucose) from example 5 in 10 ml of methylene chloride at RT and the mixture was stirred for 20 min. Subsequently, 10 ml of toluene were added and the volatile constituents were distilled off at 40° C. under reduced pressure. Two times 10 ml of toluene were further co-distilled. The residue was then taken up in 10 ml of CH₂Cl₂.

Into a second flask were placed 0.8 g of dried and ground molecular sieves (4 Å), 1.32 g (13 mmol) of triethylamine, 0.13 g of iodine (0.5 mmol), 0.07 g of NaI (0.5 mmol) and 5.49 g (10 mmol) of lactose unit from preparation example 4 in 7 ml of DCM and the mixture heated to reflux.

The solution of the protected 1-iodofucose in methylene chloride was added dropwise to the second solution and the mixture was stirred under reflux for 24 h. The reaction mixture was filtered through celite and washed with 10 ml of CH₂Cl₂. The filtrate was washed twice each with 20 ml of 10% sodium thiosulfate solution and once with 20 ml of H₂O. The organic phase was dried over Na₂SO₄, filtered and concentrated under vacuum.

The residue was chromatographed on silica gel 60 (particle size 0.04-0.063 mm, column bed volume 1200 ml) with cyclohexane/ethyl acetate 1/1 with addition of 1% triethylamine. This gave 1 g of the title compound.

¹³C-NMR(CD₂Cl₂, 500 MHz): δ (ppm) 170.97, 110.46, 110.32, 108.97, 108.57, 105.91, 101.33, 96.75, 80.50, 78.15, 77.80, 76.80, 76.71, 75.77, 75.54, 74.83, 74.18, 71.89, 71.34, 65.55, 63.65, 63.23, 56.15, 53.77, 28.73, 27.93, 27.39, 27.09, 26.84, 26.67, 26.38, 25.32, 21.03, 16.71, 0.25, 0.25, 0.25.

### Example 7

### Preparation of the Compound of the Formula (III) Where R¹═H, R²═CH₃, R³═CH₃, R^{a} and R^{b}═—C(CH₃)₂— and R^{c}═H

To a methanolic solution of 0.33 g (0.41 mmol) of the compound from example 6 were added 220 mg (4 eq.) of K₂CO₃ and the mixture stirred at RT. After 20 h, a further 55 mg (1 eq.) of K₂CO₃ were added and the mixture stirred at RT for 22 h. Subsequently, the volatile constituents were removed under reduced pressure, the residue was dissolved in 10 ml of methylene chloride and washed three times each with 10 ml of water. After drying over Na₂SO₄ and removal of the solvent under reduced pressure, 280 mg of the title compound were obtained.

### Example 8

### Preparation of 2′-O-fucosyllactose;

The crude product from example 7 was stirred in 10 ml of 0.5N HCl at RT for 16 h. The reaction mixture was subsequently concentrated. According to HPLC analysis, the title compound 2′-O-fucosyllactose was obtained which had a retention time which was identical to the retention time of an authentic reference sample of 2′-O-fucosyllactose.

### Example 9

### Preparation of 2,3,4-tri-O-benzylfucose

At 0° C., 7.6 g (94.5 mmol) of acetyl chloride were slowly added dropwise over 20 min. to 170 g of methanol. The mixture was subsequently warmed to RT and stirred at RT for 10 min. After addition of 14.92 g (90 mmol) of L-fucose, the mixture was stirred under reflux for 7 h. The reaction mixture was subsequently cooled to RT, 16.16 g (126 mmol) of Na₂CO₃ were added and stirring at RT continued for 16 h. The suspension was filtered and the solvent was removed under reduced pressure. The 1-O-methylfucose was then further reacted:

To this end, the residue was dissolved in 300 ml of DMF and 20.1 g (502.8 mmol) of sodium hydride (60%) was added portionwise. After stirring for 30 min., 87,7 g (502.8 mmol) of benzyl bromide (98%) were added dropwise and the mixture was stirred at RT for 16 h. Subsequently, 200 ml of saturated ammonium chloride solution and 200 ml of ethyl acetate were added. After stirring briefly, the phases were separated and the organic phase was washed twice each with 100 ml of H₂O. 61.6 g of crude product were obtained.

19.7 g (44 mmol) of the crude product were heated to reflux in 287 ml of 80% acetic acid in H₂O and 80 ml of 1N HCl (internal temperature: 100° C.; bath temperature: 115° C.) and the mixture was stirred under reflux for 4 h. Subsequently, the reaction mixture was cooled to RT and extracted twice with 100 ml of CH₂Cl₂. The combined organic phases were washed twice each with 50 ml of saturated NaHCO₃ solution, dried over MgSO₄ and concentrated under reduced pressure. This gave 17.3 g of the title compound as a mixture of anomers 9-I and 9-I.

Anomer 9-I:

¹³C-NMR(CD₂Cl₂, 500 MHz): δ (ppm) 139.24, 139.19, 138.84, 128.79, 128.79, 128.59, 128.59, 128.54, 128.54, 128.26, 128.26, 128.00, 127.98, 127,87, 127.85, 127.84, 127.79, 127.22, 92.03, 79.18, 78.29, 77.06, 75.41, 73.59, 72.98, 66.91, 16.94.

Anomer 9-II:

¹³C-NMR(CD₂Cl₂, 500 MHz): δ (ppm) 139.24, 139.19, 138.84, 128.79, 128.79, 128.69, 128.69, 128.59, 128.59, 128.00, 128.00, 127.98, 127.98, 127.87, 127.87, 127.84, 127.79, 127.22, 97.98, 82.79, 81.08, 77.38, 75.46, 75.22, 73.17, 70.95, 17.10.

### Example 10

### (Preparation Example): Preparation of 1-O-trimethylsilyl-2,3,4-tri-O-benzylfucopyranose

10.3 g (21.3 mmol) of 90% strength product from example 9 were dissolved in 22 g of DMF to which 2.6 g of triethylamine were added and the mixture was cooled to 0° C. 2.5 g (22.4 mmol) of chlorotrimethylsilane were then slowly added dropwise over 25 min. at −5 to 0° C. and the reaction mixture was stirred at 0° C. for 4 h. 35 ml of pentane were then added and the mixture was stirred briefly at −5 to 0° C. 23 ml of cold H₂O were then slowly added dropwise at −5 to 0° C. and after stirring briefly the phases were separated. The organic phase was washed 3 times each with 10 ml of H₂O and once with 10 ml of saturated NaCl solution and concentrated under reduced pressure. 10.5 g of the residue were chromatographed on silica gel 60 (particle size 0.04-0.063 mm, column bed volume 1200 ml). This gave 6.6 g of the title compound as a mixture (85:15) of the anomers 10-I and 10-II with a purity of 85 to 90%.

Anomer 10-I:

¹³C-NMR (CD₂Cl₂, 500 MHz): δ (ppm) 139.43, 139.42, 139.43, 128.67, 128.67, 128.59, 128.59, 128.55, 128.55, 128.48, 128.48, 128.86, 128.86, 127.86, 127,86, 127.82, 127.79, 127.73, 92.61, 79.34, 78.84, 77.57, 75.42, 73.16, 73.15, 66.44, 16.89, 0.03, 0.03, 0.03.

Anomer 10-II:

¹³C-NMR (CD₂Cl₂, 500 MHz): δ (ppm) 139.66, 139.30, 139.26, 128.71, 128.71, 128.65, 128,65, 128.57, 128.57, 127.79, 127.79, 127.76, 127.76, 127.73, 127.73, 127.71, 127.37, 126.89, 98.48, 82.65, 81.41, 77.55, 75.38, 75.20, 73.22, 70.63, 17.13, 0.27, 0.27, 0.27.

### Example 11

### Preparation of the Compound of the Formula (III) Where R¹═acetyl, R²═CH₃, R³═CH₃, R^{a}═R^{b}═R^{c}=benzyl

2.52 g (10.9 mmol) of TMSI were added to 6.5 g (10.9 mmol) of 85% product from example 10 dissolved in 10 ml of methylene chloride and the mixture was stirred for 20 min. 10 ml of toluene were then added and the volatile constituents were removed under reduced pressure and the residue co-distilled twice more with 10 ml each of toluene. The crude product was subsequently taken up in 10 ml of DMF.

A suspension of 1.3 g of ground molecular sieves (4 Angström), 5.93 g (10.9 mmol) of lactose unit from example 4, 1.43 g (14.2 mmol) of triethylamine, 0.138 g (0.55 mmol) of iodine and 0.083 g (0.55 mmol) of sodium iodide were charged in a second flask at RT and heated to 50° C. To this suspension was added dropwise the protected 1-iodofucose in DMF prepared in the first flask above and the mixture stirred at 50° C. for 24 h. The solid components of the reaction mixture were filtered off and the filtrate was washed twice each with 20 ml of 10% sodium thiosulfate solution and once with 20 ml of H₂O. The combined organic phases were concentrated at 40° C. and chromatographed on silica gel 60 (particle size 0.04-0.063 mm, column bed volume 1200 ml) with cyclohexane/ethyl acetate 1/1. This gave 130 mg of the title compound.

¹³C-NMR(CD₂Cl₂, 500 MHz): δ (ppm) 170.97, 139.79, 139.54, 139.46, 128,62, 128.62, 128.50, 128.50, 128.50, 128.50, 128.37, 128.37, 128.03, 128.03, 127.77, 127.73 127.73, 127.66, 127.65, 110.58, 110.19, 108.98, 105,93, 101.54, 95.38, 80.64, 79.34, 79.00, 78.03, 77.55, 76,92, 75.68, 75.44, 75.41, 75,31, 74.18, 73.34, 72.88, 71.24, 66.74, 65.74, 63.59, 56.21, 53.70, 27.97, 27,39, 27.15, 27.08, 26.51, 25.40, 21.03, 17.00.

### Example 12

### Preparation of the Compound of the Formula (III) Where R¹═H, R²═CH₃, R³═CH₃, R^{a}═R^{b}═R^{c}=benzyl

0.13 g (0.13 mmol) of the compound from example 11 was stirred with 38 mg of K₂CO₃ (0.26 mmol) in 10 ml of MeOH at RT for 22 h. The volatile constituents were removed under reduced pressure, the residue was dissolved in 10 ml of methylene chloride and washed with 3 ml of water. The aqueous phase was extracted twice with ethyl acetate. The combined organic phases were concentrated under reduced pressure. This gave 130 mg of the title compound.

¹³C-NMR(CD₂Cl₂, 126 MHz): δ (ppm) 139.76, 139.50, 139.46, 128.66, 128.66, 128.50, 128.50, 128.50, 128.50, 128.35, 128.35, 128.08, 128.08, 127.78, 127.74, 127.74, 127.70, 127.68, 110.70, 109.83, 109.02, 108.05, 101.93, 95.40, 81.19, 79.23, 79.04, 78.25, 77.51, 76.92, 75.82, 75.47, 75.40, 75.09, 75.01, 74,44, 73.26, 72.87, 66.66, 65.50, 62.60, 57.82, 54.63, 28.04, 27.19, 27.17, 26.93, 26.53, 25.31, 17.04.

### Example 13

### Preparation of 2′-O-fucosyllactose

0.13 g of the compound from example 12 was dissolved in 50 ml of methanol and stirred in the presence of 13 mg of 10% Pd/C at 5 bar H₂ for 24 h, filtered off and concentrated. The crude product thus obtained was then stirred in 25 ml of 0.5N HCl for 24 h, neutralized by filtering through 80 g of basic ion exchanger and concentrated under reduced pressure. The crude product is identical with an authentic sample of 2′-FL by ¹³C-NMR and HPLC.

1.-35. (canceled)

36. A method for preparing 2′-O-fucosyllactose, comprising the steps of
a) reacting a protected fucose of the general formula (I), 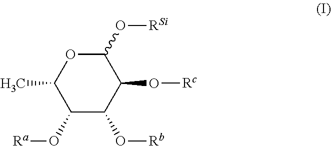
in which
R^{a} and R^{b} are the same or different and are —C(═O)—C₁-C₆-alkyl, —C(═O)-phenyl, wherein phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, or benzyl, wherein said benzyl is unsubstituted or optionally has 1, 2 or 3 substituents selected from halogen, C₁-C₄-alkyl or C₁-C₄-alkoxy, or
R^{a} and R^{b} together are a carbonyl radical —(C═O)— or a substituted methylene radical —C(R^{d}R^{e})—, wherein R^{d} and R^{e} are the same or different and are selected from hydrogen, phenyl and C₁-C₄-alkyl or both radicals R^{d} and R^{e} together are linear C₄-C₆-alkenyl,
R^{c} is a radical R^{Si} or benzyl, wherein said benzyl is unsubstituted or optionally has 1, 2 or 3 substituents selected from halogen, C₁-C₄-alkyl or C₁-C₄-alkoxy, and
R^{Si} may be the same or different and is a radical of the formula SiR^{f}R^{g}R^{h}, wherein R^{f}, R^{g} and R^{h} are the same or different and are selected from C₁-C₈-alkyl, C₃-C₈-cycloalkyl, phenyl and C₃-C₈-cycloalkyl-C₁-C₄-alkyl,
with a tri(C₁-C₆-alkyl)silyl iodide to give a protected 1-iodofucose of the general formula (I.a) 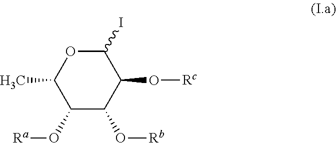
wherein R^{a}, R^{b} and R^{c} have the definitions stated above;
b) reacting the protected 1-iodofucose of the general formula (I.a) obtained in step a) with a compound of the general formula (II), 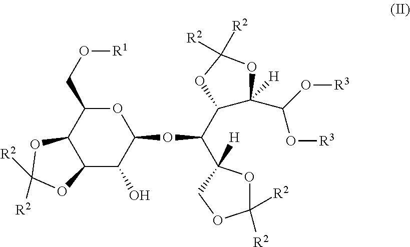
in which
R¹ is radical a C(═O)—R¹¹ or a radical SiR¹²R¹³R¹⁴, in which
R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
R¹², R¹³ and R¹⁴ are the same or different and are selected from C₁-C₈-alkyl, C₃-C₈-cycloalkyl, phenyl and C₃-C₈-cycloalkyl-C₁-C₄-alkyl,
or
is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl;
R² may be the same or different and are C₁-C₈-alkyl or two radicals R² attached to the same carbon atom together form a linear C₃-C₆-alkenyl, which is unsubstituted or has 1 to 6 methyl groups as substituents;
R³ may be the same or different and are C₁-C₈-alkyl or both radicals R³ together form a linear C₁-C₄-alkenyl, which is unsubstituted or has 1 to 6 methyl groups as substituents;
in the presence of at least one base;
c) deprotecting the coupling product of the general formula (III) obtained in step b) 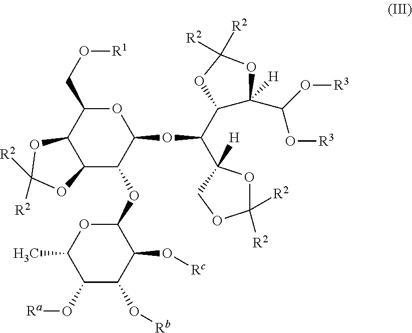
where R^{a}, R^{b}, R^{c}, R¹, R² and R³ are as defined above;
to obtain 2′-O-fucosyllactose.

37. The method of claim 36, wherein the tri(C₁-C₆-alkyl)silyl iodide used in step a) is selected from trimethylsilyl iodide.

38. The of claim 36, wherein the tri(C₁-C₆-alkyl)silyl iodide is used in an amount of 0.8 to 4 mol per mole of the compound of the formula (I).

39. The method of claim 36, wherein the tri(C₁-C₆-alkyl)silyl iodide is generated in situ by treatment of the corresponding tri(C₁-C₆-alkyl)silyl chloride with an iodide salt or by treatment of the corresponding hexaalkyl(C₁-C₆-alkyl)disilane with iodine.

40. The method of claim 39, wherein in step a) the hexaalkyl-(C₁-C₆-alkyl)disilane is firstly reacted with iodine and the reaction mixture thus obtained is subsequently reacted with compound (I).

41. The method of claim 36, wherein the base used in step b) is used in at least an equimolar amount, based on the compound of the general formula (I.a).

42. The method of claim 36, wherein the base comprises at least one base which is selected from amine bases.

43. The method of claim 42, wherein the base comprises additionally a base selected from alkali metal carbonates, alkali metal hydrogen carbonates and mixtures thereof.

44. The method according to claim 43, wherein in step
a) hexaalkyl(C₁-C₆-alkyl)disilane is firstly reacted with iodine and the reaction mixture thus obtained is subsequently reacted with the compound of the general formula (I) and in step
b) the reaction mixture obtained in step a) is reacted with a base selected from alkali metal carbonates, alkali metal hydrogen carbonates and mixtures thereof, and the mixture thus obtained is subsequently reacted with the compound of the general formula (II) in the presence of the amine base.

45. The method of claim 36, wherein step b) is carried out additionally in the presence of at least one reagent selected from iodine, iodide salts and triarylphosphine oxides and mixtures thereof.

46. The method of claim 36, wherein in step c):
c.1) the compound of the formula (III), where
R^{a} and R^{b} are the same or different and are —C(═O)—C₁-C₆-alkyl, —C(═O)-phenyl, wherein phenyl is unsubstituted or optionally has 1 to 5 substituents selected from halogen, CN, NO₂, C₁-C₄-alkyl, C₁-C₄-alkoxy, C₁-C₄-haloalkyl and C₁-C₅-haloalkoxy, or R^{a} and R^{b} together are a carbonyl radical —(C═O)—,
R^{c} is a radical R^{Si},
R^{I} is a radical —C(═O)—R¹′ or a radical SiR¹²R¹³R¹⁴, in which
R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
R¹², R¹³ and R¹⁴ are the same or different and are selected from C₁-C₈-alkyl, C₃-C₈-cycloalkyl, phenyl and C₃-C₈-cycloalkyl-C₁-C₄-alkyl,
R² may be the same or different and are C₁-C₈-alkyl or two radicals R² attached to the same carbon atom together form a linear C₃-C₆-alkenyl, which is unsubstituted or has 1 to 6 methyl groups as substituents, and
R³ may be the same or different and are C₁-C₈-alkyl or both radicals R³ together form a linear C₁-C₄-alkenyl, which is unsubstituted or has 1 to 6 methyl groups as substituents,
is firstly treated with a C₁-C₄-alkanol and an alkali metal base, wherein a compound of the formula (IIIb′) is obtained: 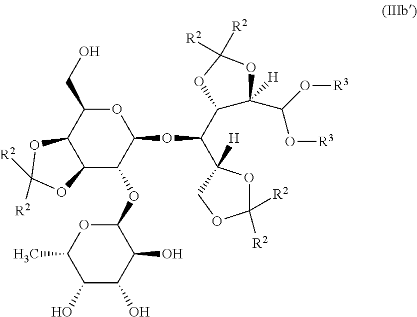
in which
R² and R³ have the definitions stated above,
and subsequently the remaining protecting groups are removed by treating the compound of the formula (IIIb′) with water in the presence of an acid;
or
c.2) the compound of the formula (III), where
R^{a} and R^{b} together are a substituted methylene radical —C(R^{d}R^{e})—, where R^{d} and R^{e} are the same or different and are selected from hydrogen, phenyl and C₁-C₄-alkyl or both radicals R^{d} and R^{e} together are linear C₄-C₆-alkenyl,
R^{c} is a radical R^{Si},
R¹ is a radical —C(═O)—R¹¹ or a radical SiR¹²R¹³R¹⁴, in which
R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
R¹², R¹³ and R¹⁴ are the same or different and are selected from C₁-C₈-alkyl, C₃-C₈-cycloalkyl, phenyl and C₃-C₈-cycloalkyl-C₁-C₄-alkyl, and
R² and R³ have the definitions stated above,
is firstly treated with a C₁-C₄-alkanol and an alkali metal base, wherein a compound of the formula (IIIb′) is obtained: 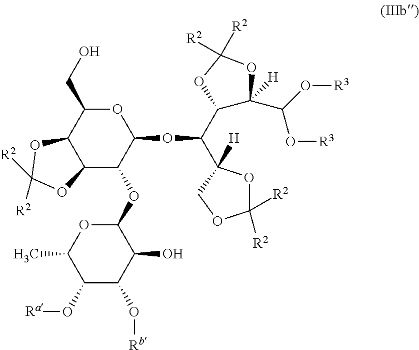
in which
R^{a}′ and R^{b}′ together are a substituted methylene radical —C(R^{d}R^{e})—, where R^{d} and R^{e} are the same or different and are selected from hydrogen, phenyl and C₁-C₄-alkyl or both radicals R^{d} and R^{e} together are linear C₄-C₆-alkenyl, and
R² and R³ have the definitions stated above,
and subsequently the remaining protecting groups are removed by treating the compound of the formula (IIIb″) with water in the presence of an acid;
c.3) the compound of the formula (III), where
R^{a} and R^{b} are the same or different and are benzyl, wherein said benzyl is unsubstituted or optionally has 1, 2 or 3 substituents selected from halogen, C₁-C₄-alkyl or C₁-C₄-alkoxy,
R^{c} is a radical R^{Si},
R¹ is a radical —C(═O)—R¹¹ or a radical SiR¹²R¹³R¹⁴, in which
R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
R¹², R¹³ and R¹⁴ are the same or different and are selected from C₁-C₈-alkyl, C₃-C₈-cycloalkyl, phenyl and C₃-C₈-cycloalkyl-C₁-C₄-alkyl, and
R² and R³ have the definitions stated above,
is firstly treated with a C₁-C₄-alkanol and an alkali metal base, wherein a compound of the formula (IIIb′″) is obtained: 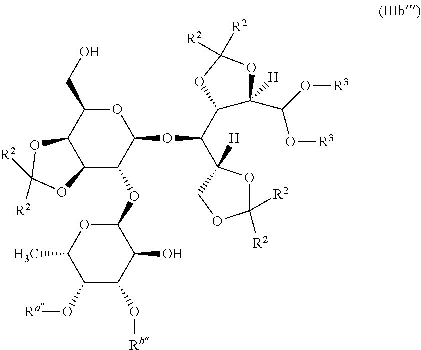
in which
R^{a}″ and R^{b}″ are the same or different and are benzyl, wherein said benzyl is unsubstituted or optionally has 1, 2 or 3 substituents selected from halogen, C₁-C₄-alkyl or C₁-C₄-alkoxy, and
R² and R³ have the definitions stated above,
subsequently the compound of the formula (IIIb′″) is treated with water in the presence of an acid, wherein a compound of the formula (IVa′) is obtained: 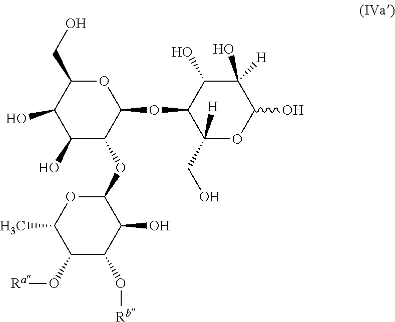
in which
R^{a}″ and R^{b}″ are the same or different and are benzyl, wherein said benzyl is unsubstituted or optionally has 1, 2 or 3 substituents selected from halogen, C₁-C₄-alkyl or C₁-C₄-alkoxy,
and subsequently the remaining benzylic protective groups are removed with hydrogen in the presence of a hydrogenation catalyst or oxidatively;
or
c.4) the compound of the formula (III), where
R^{a} and R^{b} are the same or different and are benzyl, wherein said benzyl is unsubstituted or optionally has 1, 2 or 3 substituents selected from halogen, C₁-C₄-alkyl or C₁-C₄-alkoxy,
R^{c} is benzyl, wherein said benzyl is unsubstituted or optionally has 1, 2 or 3 substituents selected from halogen, C₁-C₄-alkyl or C₁-C₄-alkoxy,
R¹ is a radical —C(═O)—R¹¹ or a radical SiR¹²R¹³R¹⁴, in which
R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
R¹², R¹³ and R¹⁴ are the same or different and are selected from C₁-C₈-alkyl, C₃-C₈-cycloalkyl, phenyl and C₃-C₈-cycloalkyl-C₁-C₄-alkyl, and
R² and R³ have the definitions stated above,
is firstly treated with a C₁-C₄-alkanol and an alkali metal base, wherein a compound of the formula (IIIc) is obtained: 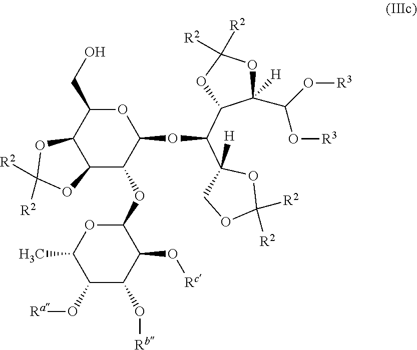
in which
R^{a}″, R^{b}″ and R^{c}′ are each independently benzyl, wherein said benzyl is unsubstituted or optionally has 1, 2 or 3 substituents selected from halogen, C₁-C₄-alkyl or C₁-C₄-alkoxy, and
R² and R³ have the definitions stated above,
subsequently the compound of the formula (IIIc) is treated with water in the presence of an acid, and subsequently the remaining benzylic protective groups are removed with hydrogen in the presence of a hydrogenation catalyst or oxidatively;
or
c.5) the compound of the formula (III), where
R^{a} and R^{b} are the same or different and are —C(═O)—C₁-C₆-alkyl, —C(═O)-phenyl, wherein phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, or R^{a} and R^{b} together are a carbonyl radical —(C═O)—,
R^{c} is benzyl, wherein said benzyl is unsubstituted or optionally has 1, 2 or 3 substituents selected from halogen, C₁-C₄-alkyl or C₁-C₄-alkoxy,
R¹ is a radical —C(═O)—R¹¹ or a radical SiR¹²R¹³R¹⁴, in which
R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
R¹², R¹³ and R¹⁴ are the same or different and are selected from C₁-C₈-alkyl, C₃-C₈-cycloalkyl, phenyl and C₃-C₈-cycloalkyl-C₁-C₄-alkyl, and
R² and R³ have the definitions stated above,
is firstly treated with a C₁-C₄-alkanol and an alkali metal base, wherein a compound of the formula (IIId) is obtained: 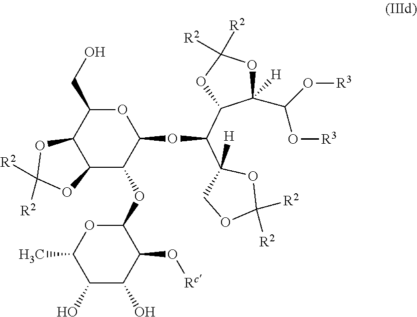
in which
R^{c}′ is benzyl, wherein said benzyl is unsubstituted or optionally has 1, 2 or 3 substituents selected from halogen, C₁-C₄-alkyl or C₁-C₄-alkoxy, and
R² and R³ have the definitions stated above,
subsequently the compound of the formula (IIId) is treated with water in the presence of an acid, and the remaining benzylic protective group is removed with hydrogen in the presence of a hydrogenation catalyst or oxidatively;
c.6) the compound of the formula (III), where
R^{a} and R^{b} together are a substituted methylene radical —C(R^{d}R^{e})—, where R^{d} and R^{e} are the same or different and are selected from hydrogen, phenyl and C₁-C₄-alkyl or both radicals R^{d} and R^{e} together are linear C₄-C₆-alkenyl,
R^{c} is benzyl, wherein said benzyl is unsubstituted or optionally has 1, 2 or 3 substituents selected from halogen, C₁-C₄-alkyl or C₁-C₄-alkoxy,
R¹ is a radical —C(═O)—R¹¹ or a radical SiR¹²R¹³R¹⁴, in which
R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
R¹², R¹³ and R¹⁴ are the same or different and are selected from C₁-C₈-alkyl, C₃-C₈-cycloalkyl, phenyl and C₃-C₈-cycloalkyl-C₁-C₄-alkyl, and
R² and R³ have the definitions stated above,
is firstly treated with a C₁-C₄-alkanol and an alkali metal base, wherein a compound of the formula (IIId′) is obtained: 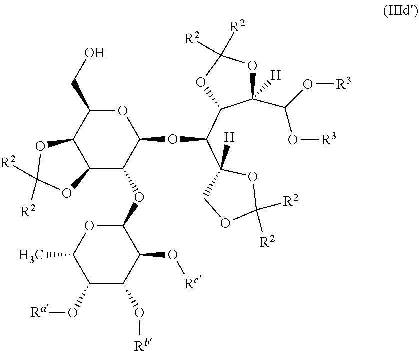
in which
R^{a}′ and R^{b}′ together are a substituted methylene radical —C(R^{d}R^{e})—, where R^{d} and R^{e} are the same or different and are selected from hydrogen, phenyl and C₁-C₄-alkyl or both radicals R^{d} and R^{e} together are linear C₄-C₆-alkenyl,
R^{c}′ is benzyl, wherein said benzyl is unsubstituted or optionally has 1, 2 or 3 substituents selected from halogen, C₁-C₄-alkyl or C₁-C₄-alkoxy, and
R² and R³ have the definitions stated above,
subsequently the compound of the formula (IIId′) is treated with water in the presence of an acid, and the remaining benzylic protective group is removed with hydrogen in the presence of a hydrogenation catalyst or oxidatively;
or
c.7) the compound of the formula (III), where
R^{a} and R^{b} are the same or different and are benzyl, wherein said benzyl is unsubstituted or optionally has 1, 2 or 3 substituents selected from halogen, C₁-C₄-alkyl or C₁-C₄-alkoxy,
R^{c} is a radical R^{Si},
R¹ is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl, and
R² and R³ have the definitions stated above,
is firstly treated with a C₁-C₄-alkanol and an alkali metal base, wherein a compound of the formula (IIIe) is obtained: 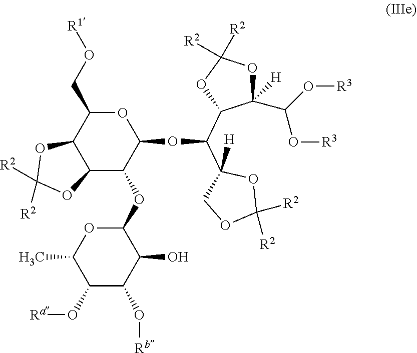
in which
R^{a}′ and R^{b}′are the same or different and are benzyl, wherein said benzyl is unsubstituted or optionally has 1, 2 or 3 substituents selected from halogen, C₁-C₄-alkyl or C₁-C₄-alkoxy,
R¹′ is benzyl, wherein said benzyl is unsubstituted or optionally has 1, 2 or 3 substituents selected from halogen, hydroxyl, C₁-C₄-alkyl or C₁-C₄-alkoxy, and
R² and R³ have the definitions stated above,
subsequently the compound of the formula (IIIe) is treated with water in the presence of an acid, wherein a compound of the formula (IVb′) is obtained: 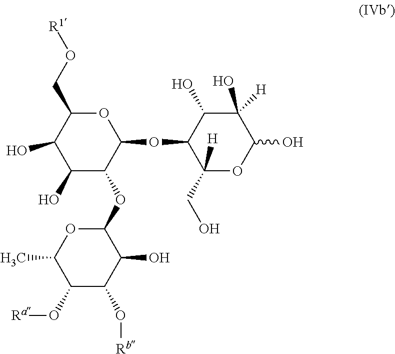
in which
R^{a}″ and R^{b}″ are the same or different and are benzyl, wherein said benzyl is unsubstituted or optionally has 1, 2 or 3 substituents selected from halogen, C₁-C₄-alkyl or C₁-C₄-alkoxy, and
R¹′ is benzyl, wherein said benzyl is unsubstituted or optionally has 1, 2 or 3 substituents selected from halogen, hydroxyl, C₁-C₄-alkyl or C₁-C₄-alkoxy,
and subsequently the remaining benzylic protective groups are removed with hydrogen in the presence of a hydrogenation catalyst or oxidatively;
or
c.8) the compound of the formula (III), where
R^{a}, R^{b} and R^{c} are the same or different and are benzyl, wherein said benzyl is unsubstituted or optionally has 1, 2 or 3 substituents selected from halogen, C₁-C₄-alkyl or C₁-C₄-alkoxy,
R¹ is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl, and
R² and R³ have the definitions stated above,
is firstly treated with hydrogen in the presence of a hydrogenation catalyst or oxidatively, wherein a compound of the formula (IIIb′) is obtained: 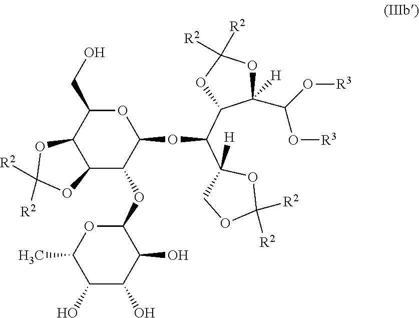
in which
R² and R³ have the definitions stated above, and
subsequently the compound of the formula (IIIb′) is treated with water in the presence of an acid;
or
c.9) the compound of the formula (III), where
R^{a} and R^{b} are the same or different and are —C(═O)—C₁-C₆-alkyl, —C(═O)-phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, or R^{a} and R^{b} together are a carbonyl radical —(C═O)—,
R^{c} is benzyl, wherein said benzyl is unsubstituted or optionally has 1, 2 or 3 substituents selected from halogen, C₁-C₄-alkyl or C₁-C₄-alkoxy,
R¹ is benzyl, wherein said benzyl is unsubstituted or optionally has 1, 2 or 3 substituents selected from halogen, C₁-C₄-alkyl, C₁-C₄-alkoxy or O—C(═O)—C₁-C₄-alkyl, and
R² and R³ have the definitions stated above,
is firstly treated with a C₁-C₄-alkanol and an alkali metal base, wherein a compound of the formula (IIIf) is obtained: 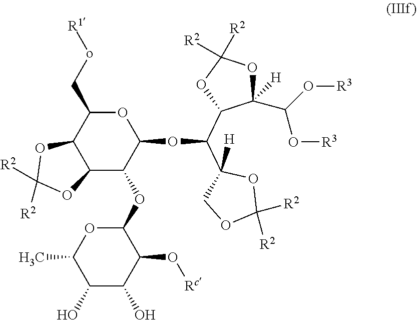
in which
R^{c}′ is benzyl, wherein said benzyl is unsubstituted or optionally has 1, 2 or 3 substituents selected from halogen, C₁-C₄-alkyl or C₁-C₄-alkoxy,
R¹′ is benzyl, wherein said benzyl is unsubstituted or optionally has 1, 2 or 3 substituents selected from halogen, hydroxyl, C₁-C₄-alkyl or C₁-C₄-alkoxy, and
R² and R³ have the definitions stated above,
subsequently the compound of the formula (IIIf) is treated with water in the presence of an acid, wherein a compound of the formula (IVc) is obtained: 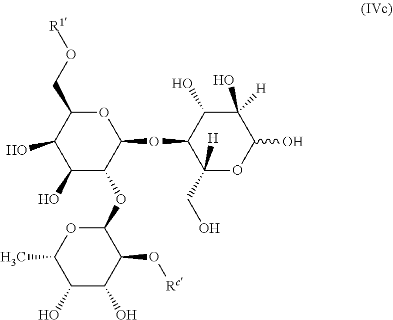
in which
R^{c}′ is benzyl, wherein said benzyl is unsubstituted or optionally has 1, 2 or 3 substituents selected from halogen, C₁-C₄-alkyl or C₁-C₄-alkoxy, and
R¹′ is benzyl, wherein said benzyl is unsubstituted or optionally has 1, 2 or 3 substituents selected from halogen, hydroxyl, C₁-C₄-alkyl or C₁-C₄-alkoxy,
and subsequently the remaining benzylic protective groups are removed with hydrogen in the presence of a hydrogenation catalyst or oxidatively;
or
c.10) the compound of the formula (III), where
R^{a} and R^{b} together are a substituted methylene radical —C(R^{d}R^{e})—, where R^{d} and R^{e} are the same or different and are selected from hydrogen, phenyl and C₁-C₄-alkyl or both radicals R^{d} and R^{e} together are linear C₄-C₆-alkenyl,
R^{c} is benzyl, wherein said benzyl is unsubstituted or optionally has 1, 2 or 3 substituents selected from halogen, C₁-C₄-alkyl or C₁-C₄-alkoxy,
R¹ is benzyl, wherein said benzyl is unsubstituted or optionally has 1, 2 or 3 substituents selected from halogen, C₁-C₄-alkyl, C₁-C₄-alkoxy or O—C(═O)—C₁-C₄-alkyl, and
R² and R³ have the definitions stated above,
is firstly treated with water in the presence of an acid, wherein a compound of the formula (IVc) is obtained: 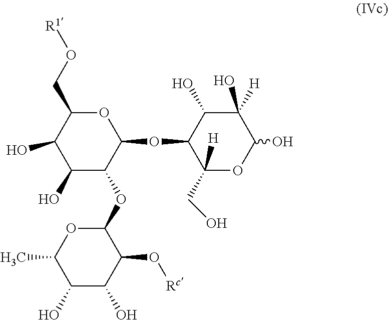
in which
R^{c}′ is benzyl, wherein said benzyl is unsubstituted or optionally has 1, 2 or 3 substituents selected from halogen, C₁-C₄-alkyl or C₁-C₄-alkoxy, and
R¹′ is benzyl, wherein said benzyl is unsubstituted or optionally has 1, 2 or 3 substituents selected from halogen, hydroxyl, C₁-C₄-alkyl or C₁-C₄-alkoxy,
and subsequently the remaining benzylic protective groups are removed with hydrogen in the presence of a hydrogenation catalyst or oxidatively.

47. The method of claim 36, wherein the radicals R^{a} and R^{b} in the formula (I) are benzyl, wherein benzyl is unsubstituted or optionally has 1, 2 or 3 substituents selected from halogen, C₁-C₄-alkoxy or —O—C(═O)—C₁-C₄-alkyl.

48. The method of claim 36, wherein the radicals R^{a} and R^{b} in the formula (I), (I.a) and (III) are acetyl, pivaloyl, benzoyl, 4-chlorobenzoyl or 4-fluorobenzoyl.

49. The method of claim 36, wherein the radical R^{c} in the formula (I) is a radical SiR^{f}R^{g}R^{h} , where R^{f}, R^{g} and R^{h} are the same or different and are C₁-C₄-alkyl, or is benzyl, wherein said benzyl is unsubstituted or optionally has 1 or 2 substituents selected from fluorine, chlorine, bromine, methyl and methoxy.

50. The method of claim 36, wherein the radical R^{Si}in the formula (I) is trimethylsilyl.

51. The method of claim 36, wherein the radical R¹ in the formulae (II) and (III) is selected from the group consisting of acetyl, pivaloyl, benzoyl or 4-chlorobenzoyl a radical C(═O)—R¹¹, where R¹¹ is hydrogen, C₁-C₄-alkyl, C₁-C₄-haloalkyl, phenyl or 4-chlorophenyl, R² is methyl and R³ is methyl.

52. A compound which is selected from the group consisting of
a compound of the general formula (IIIa) 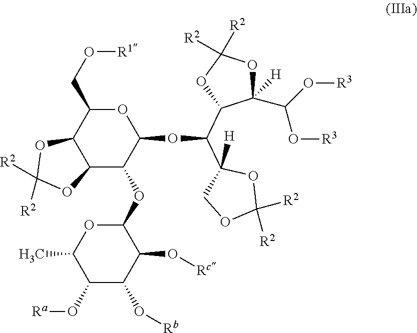
in which
R^{a}, R^{b}, R² and R³ are as defined in claim 36,
R^{c}″ is hydrogen or a radical R^{Si},
R¹″ is hydrogen, a radical —C(═O)—R¹¹ or a radical SiR¹²R¹³R¹⁴, in which
R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
R¹², R¹³ and R¹⁴ are the same or different and are selected from C₁-C₈-alkyl, C₃-C₈-cycloalkyl, phenyl and C₃-C₈-cycloalkyl-C₁-C₄-alkyl,
or
is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl, and
R² and R³ have the definitions stated in claim 36;
a compound of the general formula (IIIb) 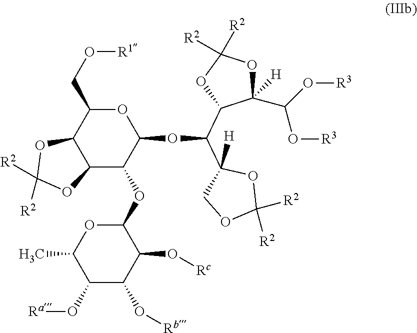
in which
R^{a}′″ and R^{b}′″ together are a carbonyl radical —(C═O)— or a substituted methylene radical —C(R^{d}R^{e})—, wherein R^{d} and R^{e} are the same or different and are selected from hydrogen, phenyl and C₁-C₄-alkyl or both radicals R^{d} and R^{e} together are linear C₄-C₆-alkenyl,
R^{c}has the definitions stated in claim 36,
R¹″ is hydrogen, a radical —C(═O)—R¹¹ or a radical SiR¹²R¹³R¹⁴, where
R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, and
R¹², R¹³ and R¹⁴ are the same or different and are selected from C₁-C₈-alkyl, C₃-C₈-cycloalkyl, phenyl and C₃-C₈-cycloalkyl-C₁-C₄-alkyl,
or
is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl, and
R² and R³ have the definitions stated in claim 36;
a compound of the general formula (IVa) 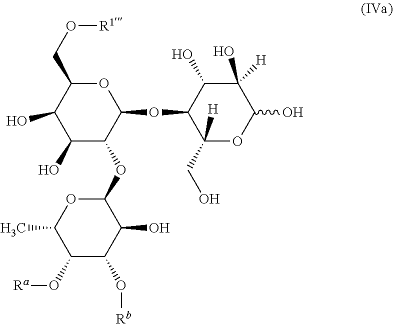
in which
R^{a} and R^{b} have the definitions stated in claim 36,
R¹′″ is hydrogen, a radical —C(═O)—R¹¹, where
R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy,
or
is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl.
and a compound of the general formula (IVb) 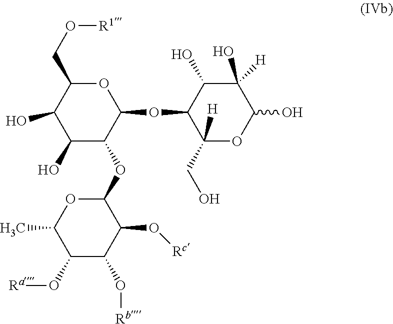
in which
R^{a}′″ and R^{b}′″ together are a carbonyl radical —(C═O)—,
R^{c}′ is benzyl which is unsubstituted or optionally has 1, 2 or 3 substituents selected from halogen, C₁-C₄-alkyl or C₁-C₄-alkoxy,
R¹′″ is hydrogen, a radical —C(═O)—R¹¹, where
R¹¹ is hydrogen, C₁-C₈-alkyl, C₁-C₈-haloalkyl, C₃-C₈-cycloalkyl, C₃-C₈-cycloalkyl-C₁-C₄-alkyl or phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy,
or
is benzyl, wherein said benzyl is unsubstituted or optionally has 1, 2 or 3 substituents selected from halogen, C₁-C₄-alkyl, C₁-C₄-alkoxy or —O—C(═O)—C₁-C₄-alkyl.

53. A compound of the general formula (I′) 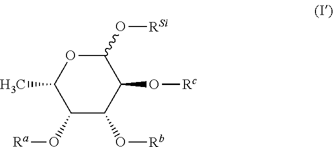
in which
R^{a} and R^{b} are the same or different and are —C(═O)—C₁-C₆-alkyl, —C(═O)-phenyl, wherein phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-alkoxy, or benzyl, wherein said benzyl is unsubstituted or optionally has 1, 2 or 3 substituents selected from halogen, C₁-C₄-alkyl or C₁-C₄-alkoxy, or R^{a} and R^{b} together are a carbonyl radical —(C═O)— or a substituted methylene radical —C(R^{d}R^{e})—, wherein R^{d} and R^{e} are the same or different and are selected from hydrogen, phenyl and C₁-C₄-alkyl or both radicals R^{d} and R^{e} together are linear C₄-C₆-alkenyl,
R^{c} is a radical R^{Si} or benzyl, wherein said benzyl is unsubstituted or optionally has 1, 2 or 3 substituents selected from halogen or C₁-C₄-alkyl, C₁-C₄-alkoxy,
R^{Si} may be the same or different and is a radical of the formula SiR^{f}R^{g}R^{h}, wherein R^{f}, R^{g} and R^{h} are the same or different and are selected from C₁-C₈-alkyl, C₃-C₈-cycloalkyl, phenyl and C₃-C₈-cycloalkyl-C₁-C₄-alkyl,
wherein the radicals R^{a}, R^{b} and R^{c} are not all three simultaneously benzyl or 4-methoxybenzyl.

54. A compound of the general formula (I′) of claim 53, selected from
1-O-trimethylsilyl-2,3,4-tri-O-4-C₁-benzylfucopyranose,
1-O-trimethylsilyl-2,3,4-tri-O-4-C₁-benzylfucopyranose,
1-O-trimethylsilyl-2,3,4-tri-O-4-Me-benzylfucopyranose,
1-O-trimethylsilyl-2,3,4-tri-O-(2,4-Cl-benzyl)fucopyranose,
1-O-trimethylsilyl-2-benzyl-3,4-di-O-4-Cl-benzylfucopyranose,
1-O-trimethylsilyl-2-benzyl-3,4-di-O-4-Me-benzylfucopyranose,
1-O-trimethylsilyl-2-benzyl-3,4-di-O-4-OMe-benzylfucopyranose,
1-O-trimethylsilyl-2-benzyl-3,4-di-O-(2,4-Cl-benzyl)fucopyranose,
1-O-trimethylsilyl-2-(4-Cl-benzyl)-3,4-di-O-4-benzylfucopyranose,
1-O-trimethylsilyl-2-(4-Cl-benzyl)-3,4-di-O-4-Cl-benzylfucopyranose,
1-O-trimethylsilyl-2-(4-Cl-benzyl)-3,4-di-O-4-Me-benzylfucopyranose,
1-O-trimethylsilyl-2-(4-Cl-benzyl)-3,4-di-O-4-OMe-benzylfucopyranose,
1-O-trimethylsilyl-2-(4-Cl-benzyl)-3,4-di-O-(2,4-Cl-benzyl)fucopyranose,
1-O-trimethylsilyl-2-(4-Me-benzyl)-3,4-di-O-4-benzylfucopyranose,
1-O-trimethylsilyl-2-(4-Me-benzyl)-3,4-di-O-4-Cl-benzylfucopyranose,
1-O-trimethylsilyl-2-(4-Me-benzyl)-3,4-di-O-4-Me-benzylfucopyranose,
1-O-trimethylsilyl-2-(4-Me-benzyl)-3,4-di-O-4-OMe-benzylfucopyranose,
1-O-trimethylsilyl-2-(4-Me-benzyl)-3,4-di-O-(2,4-Cl-benzyl)fucopyranose,
1-O-trimethylsilyl-2-(4-OMe-benzyl)-3,4-di-O-4-benzylfucopyranose,
1-O-trimethylsilyl-2-(4-OMe-benzyl)-3,4-di-O-4-Cl-benzylfucopyranose,
1-O-trimethylsilyl-2-(4-OMe-benzyl)-3,4-di-O-4-Me-benzylfucopyranose,
1-O-trimethylsilyl-2-(4-OMe-benzyl)-3,4-di-O-4-OMe-benzylfucopyranose,
1-O-trimethylsilyl-2-(4-OMe-benzyl)-3,4-di-O-(2,4-Cl-benzyl)fucopyranose,
1,2-di-O-trimethylsilyl-3,4-di-O-benzylfucopyranose,
1,2-di-O-trimethylsilyl-3,4-di-O-4-Cl-benzylfucopyranose,
1,2-di-O-trimethylsilyl-3,4-di-O-4-F-benzylfucopyranose,
1,2-di-O-trimethylsilyl-3,4-di-O-4-Me-benzylfucopyranose,
1,2-di-O-trimethylsilyl-3,4-di-O-4-OMe-benzylfucopyranose,
1,2-di-O-trimethylsilyl-3,4-di-O-(2,4-Cl-benzyl)fucopyranose,
1-O-trimethylsilyl-2-benzyl-3,4-di-O-benzoylfucopyranose,
1-O-trimethylsilyl-2-benzyl-3,4-di-O-(4-Cl-benzoyl)fucopyranose,
1-O-trimethylsilyl-2-benzyl-3,4-di-O-(4-F-benzoyl)fucopyranose,
1-O-trimethylsilyl-2-benzyl-3,4-di-O-4-Me-benzoylfucopyranose,
1-O-trimethylsilyl-2-benzyl-3,4-di-O-4-OMe-benzoylfucopyranose,
1-O-trimethylsilyl-2-benzyl-3,4-di-O-(2,4-Cl-benzoyl)fucopyranose,
1-O-trimethylsilyl-2-(4-Cl-benzyl)-3,4-di-O-benzoylfucopyranose,
1-O-trimethylsilyl-2-(4-Cl-benzyl)-3,4-di-O-4-Cl-benzoylfucopyranose,
1-O-trimethylsilyl-2-(4-Cl-benzyl)-3,4-di-O-4-F-benzoylfucopyranose,
1-O-trimethylsilyl-2-(4-Cl-benzyl)-3,4-di-O-4-Me-benzoylfucopyranose,
1-O-trimethylsilyl-2-(4-Cl-benzyl)-3,4-di-O-4-OMe-benzoylfucopyranose,
1-O-trimethylsilyl-2-(4-Cl-benzyl)-3,4-di-O-(2,4-Cl-benzoyl)fucopyranose,
1-O-trimethylsilyl-2-(4-Me-benzyl)-3,4-di-O-benzoylfucopyranose,
1-O-trimethylsilyl-2-(4-Me-benzyl)-3,4-di-O-4-Cl-benzoylfucopyranose,
1-O-trimethylsilyl-2-(4-Me-benzyl)-3,4-di-O-4-F-benzoylfucopyranose,
1-O-trimethylsilyl-2-(4-Me-benzyl)-3,4-di-O-4-Me-benzoylfucopyranose,
1-O-trimethylsilyl-2-(4-Me-benzyl)-3,4-di-O-4-OMe-benzoylfucopyranose,
1-O-trimethylsilyl-2-(4-Me-benzyl)-3,4-di-O-(2,4-Cl-benzoyl)fucopyranose,
1-O-trimethylsilyl-2-(4-OMe-benzyl)-3,4-di-O-benzoylfucopyranose,
1-O-trimethylsilyl-2-(4-OMe-benzyl)-3,4-di-O-4-Cl-benzoylfucopyranose,
1-O-trimethylsilyl-2-(4-OMe-benzyl)-3,4-di-O-4-F-benzoylfucopyranose,
1-O-trimethylsilyl-2-(4-OMe-benzyl)-3,4-di-O-4-Me-benzoylfucopyranose,
1-O-trimethylsilyl-2-(4-OMe-benzyl)-3,4-di-O-4-OMe-benzoylfucopyranose,
1-O-trimethylsilyl-2-(4-OMe-benzyl)-3,4-di-O-(4-Cl-benzoyl)fucopyranose,
1,2-di-O-trimethylsilyl-3,4-di-O-benzoylfucopyranose,
1,2-di-O-trimethylsilyl-3,4-di-O-(4-Cl-benzoyl)fucopyranose,
1,2-di-O-trimethylsilyl-3,4-di-O-(4-F-benzoyl)fucopyranose,
1,2-di-O-trimethylsilyl-3,4-di-O-4-Me-benzoylfucopyranose,
1,2-di-O-trimethylsilyl-3,4-di-O-4-OMe-benzoylfucopyranose,
1,2-di-O-trimethylsilyl-3,4-di-O-(2,4-Cl-benzoyl)fucopyranose.

55. A compound of the general formula (I.a′) 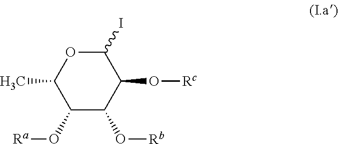
in which
R^{a} and R^{b} are the same or different and are —C(═O)—C₁-C₆-alkyl, —C(═O)-phenyl, wherein said phenyl is unsubstituted or optionally has 1 to 5 substituents selected from halogen, CN, NO₂, C₁-C₄-alkyl, C₁-C₄-alkoxy, C₁-C₄-haloalkyl and C₁-C₄-haloalkoxy, or benzyl, wherein said benzyl is unsubstituted or optionally has 1, 2 or 3 substituents selected from halogen, C₁-C₄-alkyl or C₁-C₄-alkoxy, or R^{a} and R^{b} together are a carbonyl radical —(C═O)— or a substituted methylene radical —C(R^{d}R^{e})—, wherein R^{d} and R^{e} are the same or different and are selected from hydrogen, phenyl and C₁-C₄-alkyl or both radicals R^{d} and R^{e} together are linear C₄-C₆-alkenyl,
R^{c} is a radical R^{Si} or benzyl, wherein benzyl is unsubstituted or optionally has 1, 2 or 3 substituents selected from halogen, C₁-C₄-alkyl or C₁-C₄-alkoxy,
R^{Si}may be the same or different and is a radical of the formula SiR^{f}R^{g}R^{h}, wherein R^{f}, R^{g} and R^{h} are the same or different and are selected from C₁-C₈-alkyl, C₃-C₈-cycloalkyl, phenyl and C₃-C₈-cycloalkyl-C₁-C₄-alkyl,
wherein the radicals R^{a}, R^{b} and R^{c} are not all three simultaneously benzyl and, in the case that R^{a} and R^{b} together form a dimethylmethylene radical C(CH₃CH₃)—, R^{c} is not a tert-butyldimethylsilyl radical.

56. A compound of the general formula (I.a′) of claim 55, selected from the group consisting of
1-deoxy-2,3,4-tri-O-4-Cl-benzylfucopyranosyl iodide,
1-deoxy-2,3,4-tri-O-2-Cl-benzylfucopyranosyl iodide,
1-deoxy-2,3,4-tri-O-4-Me-benzylfucopyranosyl iodide,
1-deoxy-2,3,4-tri-O-4-OMe-benzylfucopyranosyl iodide,
1-deoxy-2,3,4-tri-O-(2,4-Cl-benzyl)fucopyranosyl iodide,
1-deoxy-2-benzyl-3,4-di-O-4-Cl-benzylfucopyranosyl iodide,
1-deoxy-2-benzyl-3,4-di-O-4-Me-benzylfucopyranosyl iodide,
1-deoxy-2-benzyl-3,4-di-O-4-OMe-benzylfucopyranosyl iodide,
1-deoxy-2-benzyl-3,4-di-O-(2,4-Cl-benzyl)fucopyranosyl iodide,
1-deoxy-2-(4-Cl-benzyl)-3,4-di-O-4-benzylfucopyranosyl iodide,
1-deoxy-2-(4-Cl-benzyl)-3,4-di-O-4-Cl-benzylfucopyranosyl iodide,
1-deoxy-2-(4-Cl-benzyl)-3,4-di-O-4-Me-benzylfucopyranosyl iodide,
1-deoxy-2-(4-Cl-benzyl)-3,4-di-O-4-OMe-benzylfucopyranosyl iodide,
1-deoxy-2-(4-Cl-benzyl)-3,4-di-O-(2,4-Cl-benzyl)fucopyranosyl iodide,
1-deoxy-2-(4-Me-benzyl)-3,4-di-O-4-benzylfucopyranosyl iodide,
1-deoxy-2-(4-Me-benzyl)-3,4-di-O-4-Cl-benzylfucopyranosyl iodide,
1-deoxy-2-(4-Me-benzyl)-3,4-di-O-4-Me-benzylfucopyranosyl iodide,
1-deoxy-2-(4-Me-benzyl)-3,4-di-O-4-OMe-benzylfucopyranosyl iodide,
1-deoxy-2-(4-Me-benzyl)-3,4-di-O-(2,4-Cl-benzyl)fucopyranosyl iodide,
1-deoxy-2-(4-OMe-benzyl)-3,4-di-O-4-benzylfucopyranosyl iodide,
1-deoxy-2-(4-OMe-benzyl)-3,4-di-O-4-Cl-benzylfucopyranosyl iodide,
1-deoxy-2-(4-OMe-benzyl)-3,4-di-O-4-Me-benzylfucopyranosyl iodide,
1-deoxy-2-(4-OMe-benzyl)-3,4-di-O-4-OMe-benzylfucopyranosyl iodide,
1-deoxy-2-(4-OMe-benzyl)-3,4-di-O-(2,4-Cl-benzyl)fucopyranosyl iodide,
1-deoxy-2-O-trimethylsilyl-3,4-di-O-benzylfucopyranosyl iodide,
1-deoxy-2-O-trimethylsilyl-3,4-di-O-4-Cl-benzylfucopyranosyl iodide,
1-deoxy-2-O-trimethylsilyl-3,4-di-O-4-Me-benzylfucopyranosyl iodide,
1-deoxy-2-O-trimethylsilyl-3,4-di-O-4-OMe-benzylfucopyranosyl iodide,
1-deoxy-2-O-trimethylsilyl-3,4-di-O-(2,4-Cl-benzyl)fucopyranosyl iodide,
1-deoxy-2-benzyl-3,4-di-O-benzoylfucopyranosyl iodide,
1-deoxy-2-benzyl-3,4-di-O-(4-Cl-benzoyl)fucopyranosyl iodide,
1-deoxy-2-benzyl-3,4-di-O-(4-F-benzoyl)fucopyranosyl iodide,
1-deoxy-2-benzyl-3,4-di-O-4-Me-benzoylfucopyranosyl iodide,
1-deoxy-2-benzyl-3,4-di-O-4-OMe-benzoylfucopyranosyl iodide,
1-deoxy-2-benzyl-3,4-di-O-(2,4-Cl-benzoyl)fucopyranosyl iodide,
1-deoxy-2-(4-C1-benzyl)-3,4-di-O-benzoylfucopyranosyl iodide,
1-deoxy-2-(4-Cl-benzyl)-3,4-di-O-4-Cl-benzoylfucopyranosyl iodide,
1-deoxy-2-(4-Cl-benzyl)-3,4-di-O-4-F-benzoylfucopyranosyl iodide,
1-deoxy-2-(4-Cl-benzyl)-3,4-di-O-4-Me-benzoylfucopyranosyl iodide,
1-deoxy-2-(4-Cl-benzyl)-3,4-di-O-4-OMe-benzoylfucopyranosyl iodide,
1-deoxy-2-(4-Cl-benzyl)-3,4-di-O-(2,4-Cl-benzoyl)fucopyranosyl iodide,
1-deoxy-2-(4-Me-benzyl)-3,4-di-O-benzoylfucopyranosyl iodide,
1-deoxy-2-(4-Me-benzyl)-3,4-di-O-4-Cl-benzoylfucopyranosyl iodide,
1-deoxy-2-(4-Me-benzyl)-3,4-di-O-4-F-benzoylfucopyranosyl iodide,
1-deoxy-2-(4-Me-benzyl)-3,4-di-O-4-Me-benzoylfucopyranosyl iodide,
1-deoxy-2-(4-Me-benzyl)-3,4-di-O-4-OMe-benzoylfucopyranosyl iodide,
1-deoxy-2-(4-Me-benzyl)-3,4-di-O-(2,4-Cl-benzoyl)fucopyranosyl iodide,
1-deoxy-2-(4-OMe-benzyl)-3,4-di-O-benzoylfucopyranosyl iodide,
1-deoxy-2-(4-OMe-benzyl)-3,4-di-O-4-Cl-benzoylfucopyranosyl iodide,
1-deoxy-2-(4-OMe-benzyl)-3,4-di-O-4-F-benzoylfucopyranosyl iodide,
1-deoxy-2-(4-OMe-benzyl)-3,4-di-O-4-Me-benzoylfucopyranosyl iodide,
1-deoxy-2-(4-OMe-benzyl)-3,4-di-O-4-OMe-benzoylfucopyranosyl iodide,
1-deoxy-2-(4-OMe-benzyl)-3,4-di-O-(2,4-Cl-benzoyl)fucopyranosyl iodide,
1-deoxy-2-O-trimethylsilyl-3,4-di-O-benzoylfucopyranosyl iodide,
1-deoxy-2-O-trimethylsilyl-3,4-di-O-(4-Cl-benzoyl)fucopyranosyl iodide,
1-deoxy-2-O-trimethylsilyl-3,4-di-O-(4-F-benzoyl)fucopyranosyl iodide,
1-deoxy-2-O-trimethylsilyl-3,4-di-O-4-Me-benzoylfucopyranosyl iodide,
1-deoxy-2-O-trimethylsilyl-3,4-di-O-4-OMe-benzoylfucopyranosyl iodide, and
1-deoxy-2-O-trimethylsilyl-3,4-di-O-(2,4-Cl-benzoyl)fucopyranosyl iodide.
